(19) United States
(12) Patent Application Publication
(10) Pub. No.: US 2024/0287500 A1
(43) Pub. Date: Aug. 29, 2024

(54) Tools and Methods for Mycoplasma Engineering

(71) Applicants: FUNDACIÓ CENTRE DE REGULACIÓ GENÒMICA, Barcelona (ES); INSTITUCIÓ CATALANA DE RECERCA I ESTUDIS AVANÇATS, Barcelona (ES)

(72) Inventors: Carlos Piñero Lambea, Madrid (ES); Maria Lluch Senar, Barcelona (ES); Luis Serrano Pubul, Barcelona (ES)

(21) Appl. No.: 17/796,022

(22) PCT Filed: Jan. 29, 2021
(86) PCT No.: PCT/EP2021/052110

§ 371 Date: Jul. 28, 2022

(30) Foreign Application Priority Data
Jan. 29, 2020 (EP) 20382051.9

(51) Int. Cl. C12N 15/10; C12N 9/12; C12N 9/22

(52) U.S. Cl. CPC C12N 15/102; C12N 9/1241; C12N 9/22; C12N 2310/20

(57) ABSTRACT

The present invention concerns oligonucleotide modification systems to enable efficient genome engineering in Mycoplasma bacteria. Also intended is the use of these systems in Mycoplasma engineering, and methods comprising administering the oligonucleotide modification systems to Mycoplasma to generate genetically engineered Mycoplasma strains not naturally occurring.

### CROSS REFERENCE TO RELATED APPLICATIONS

This application is a national phase entry under 35 U.S.C. § 371 of International Patent Application PCT/EP2021/052110, filed Jan. 29, 2021, designating the United States of America and published in English as International Patent Publication WO 2021/152092 on Aug. 5, 2021, which claims the benefit under Article 8 of the Patent Cooperation Treaty to European Patent Application Serial No. 20382051.9, filed Jan. 29, 2020, the entireties of which are hereby incorporated by reference.

### FIELD OF THE INVENTION

Aspects of the invention relate to tools and genome engineering methods in the molecular biology field to genetically engineer Mycoplasma bacteria.

### BACKGROUND OF THE INVENTION

Emerging genome engineering technologies are profoundly expanding the toolbox to devise innovative ways to tackle problems in a plethora of technical fields. More specifically, genome engineering is enabling the generation of genetically modified cells or organisms that display different and favorable traits when compared to their naturally occurring counterpart. This outlook has spurred a great interest in rationally designing genetically modified organisms that may directly or indirectly help in the treatment or prevention of diseases, in a field coined synthetic biology (Serrano, Synthetic biology: promises and challenges, Molecular Systems Biology, 2007). Since synthetic biology aims to design new living forms from an engineering perspective ideally a genomic backbone is generated that is void of any irrelevant functions for the envisaged purpose of the designer organism and novel functionalities are embedded in the genome. An essential element for accomplishing this is the availability of tools and protocols that allow targeted, efficient and straightforward generation of such organisms. While many cell types of different origins can be readily modified by existing protocols, several bacteria that are of particular interest for the medical biotechnology community cannot efficiently be modified by these protocols.

One genus of bacteria of particular interest for genetic engineering is the Mycoplasma genus. The Mycoplasma genus comprises a group of bacteria sharing as distinctive features the lack of cell wall, a streamlined genome that results in limited biosynthetic capabilities, and a variant genetic code in which UGA encodes for tryptophan, rather than being read as a Stop codon (Razin et al., Molecular biology and pathogenicity of Mycoplasmas, Microbiology and Molecular Biology Reviews, 1998). All these features might be of interest for synthetic biology concerns such as orthogonality, biosafety and limited horizontal gene transfer. Furthermore, Mycoplasma species have small genome sizes (0.5 megabases (Mb) to 1.5 Mb) and this biological simplicity has proven to be beneficial for multiple areas of research including proteomics, metabolomics, systems biology and synthetic biology. Mycoplasma furthermore holds additional benefits compared to traditional organisms such as Escherichia coli or Saccharomyces cerevisiae for synthetic biology, since the popularity of these organisms to serve as designer organism is historically based on their ease of growth in laboratory conditions and high recombination efficiencies.

Along the species belonging to the Mycoplasma genus the human pathogen Mycoplasma pneumoniae arises as a promising candidate for synthetic biology projects, since it is one of the most deeply characterized living forms as a consequence of Mycoplasma being a prevalent model organism for Systems Biology for over a decade (Güell et al., Transcriptome complexity in a genome-reduced bacterium, Science, 2009, and Maier et al., Quantification of mRNA and protein and integration with protein turnover in a bacterium. Molecular Systems Biology, 2014). Thus, by removing the few and well-characterized pathogenicity determinants found in its genome (He et al., Insights into the pathogenesis of Mycoplasma pneumoniae, Molecular Medicine Reports, 2008), M pneumoniae could become an attractive genomic platform for plugging in additional functionalities to provide organisms with specific traits tailored to very diverse applications. However, the transition ofM. pneumoniae from a systems biology model organism to a suitable chassis strain for synthetic biology has been blocked so far by the historical paucity of efficient genome editing tools for Mycoplasma bacteria.

Over time, Haystack mutagenesis has become the standard method for obtaining genomically engineered Mycoplasma bacteria (Halbedel and Stulke, Tools for the genetic analysis of Mycoplasma, International Journal of Medical Biotechnology, 2007). In this technique, clones carrying transposon insertions at a locus of interest are isolated by using a comprehensive and iterative PCR screening of an ordered collection of pooled random transposon insertion mutants. However, the technique merely allows the selection of clones in which a particular gene has been disrupted, not deleted or edited. As an alternative approach, chemical synthesis (i.e. genome writing) of complete Mycoplasma genomes has been described (Gibson et al., Complete chemical synthesis, assembly, and cloning of a Mycoplasma genitalium genome, Science, 2008), however the financial resources and high level of expertise that are required impede a widespread adoption. More recently, a Mycoplasma genome engineering method was described wherein the Mycoplasma genome is temporarily transferred into yeast to allow engineering of said genome (Ruiz et al., CReasPy-Cloning: A Method for Simultaneous Cloning and Engineering of Megabase-Sized Genomes in Yeast Using the CRISPR-Cas9 System, ACS Synthetic Biology 2019). However, while transfer of a Mycoplasma genome into yeast is feasible, the edited Mycoplasma genome needs to be transferred back to a Mycoplasma recipient cell by using a technique called genome transplantation. However, to date genome transplantation is not available for Mycoplasma species that do not belong to the mycoides cluster (Labroussaa et al., Impact of donor-recipient phylogenetic distance on bacterial genome transplantation, Nucleic Acids Research, 2016). Hence, the requirement of genome transplantation renders the technique currently not suited for obtaining Mycoplasma pneumoniae bacteria.

Oligo recombineering has become a promising technique to perform gene editing in species that have been traditionally recalcitrant to standard genome engineering techniques. However it is widely accepted that the performance of ssDNA recombinases is tightly related with the phylogenetical distance between the microorganism that is being edited and the source from which the recombinase has been obtained (Sun et al., A high-efficiency recombineering system with PCR-based ssDNA in Bacillus subtilis mediated by the native phage recombinase GP35, Applied Microbiology and Biotechnology, 2015; Aparicio et al., CRISPR/Cas9-enhanced ssDNA recombineering for Pseudomonas putida, Microbiol Biotechnology, 2019; and WO 2018/081535 A2). Given that the Mycoplasma pangenome does not code for any ssDNA recombinase, oligo recombineering does not seem to be a feasible option to perform genome editing in this bacterium. Indeed, previous attempts to employ ssDNA recombinases to perform gene editing in strains different from its native source, have produced neglectable results (see for instance the performance of GP35 a ssDNA recombinase native to B. subtilis, when operating in E. coli: Datta el al., Identification and analysis of recombineering functions from Gram-negative and Gram-positive bacteria and their phages, PNAS, 2008).

There is thus an unmet need for tools and methods that allow efficient and precise genome engineering in Mycoplasma.

### SUMMARY OF THE INVENTION

As evidenced in the examples which illustrate certain representative embodiments of the invention, the present invention relates to tools and methods for efficiently modifying the genome of Mycoplasma bacteria, hereby eliminating a tedious screening procedure to identify Mycoplasma bacteria that have been modified in a desired manner. Furthermore, the inventors have defined sets of nucleotide arrangements that are particularly useful to accomplish this. The invention thus addresses the unmet need for an improved and efficient genome engineering method and related tools in Mycoplasma bacteria. The system relies on the use of a DNA binding protein, preferably a GP35 recombinase to recombine at least one exogenously supplied nucleotide arrangement (i.e. nucleotide sequence) into the genome of Mycoplasma. As will be apparent from the present disclosure as a whole, the GP35 may be introduced into Mycoplasma by any means deemed appropriate by a skilled person, including nucleotide-encoded GP35 recombinase such as DNA or RNA nucleotide sequences, GP35 recombinase protein as such, or any combinations thereof.

The invention therefore provides the following aspects:
Aspect 1. An oligonucleotide modification system comprising:
   1) a first nucleotide or amino acid arrangement comprising
(i) a promoter or a functional variant or fragment thereof which is active in Mycoplasma bacteria, operably linked to a DNA nucleotide sequence encoding a GP35 recombinase, and/or
(ii) an RNA sequence encoding a GP35 recombinase, and/or
(iii) a GP35 recombinase protein, and
   2) a second nucleotide arrangement comprising a naturally occurring Mycoplasma sequence with a minimum length of 5 nucleotides.
Aspect 2. The oligonucleotide modification system according to aspect 1, wherein the naturally occurring Mycoplasma sequence is a sequence having a minimum length of 5 nucleotides.
Aspect 3. The oligonucleotide system according to aspect 2, wherein the naturally occurring Mycoplasma sequence is a sequence has a length of from 5 to 10000 nucleotides, preferably from 15 to 7500 nucleotides, more preferably from 50 to 5000 nucleotides, even more preferably from 50 to 2500 nucleotides, even more preferably from 50 to 1000 nucleotides, yet even more preferably from 50 to 500 nucleotides.
Aspect 4. An oligonucleotide modification system comprising:
1) a first nucleotide or amino acid arrangement comprising
   (i) a promoter or a functional variant or fragment thereof which is active in Mycoplasma bacteria, operably linked to a nucleotide sequence encoding a GP35 recombinase, or
   (ii) an RNA sequence encoding a GP35 recombinase, or
   (iii) a GP35 recombinase protein, and
2) a second nucleotide arrangement comprising a naturally occurring Mycoplasma sequence, wherein said naturally occurring Mycoplasma sequence is comprised as two non-adjacent nucleotide sequences in the second nucleotide arrangement, each having a minimum length of 5 nucleotides.
Aspect 5. An oligonucleotide modification system comprising:
   1) a first nucleotide arrangement comprising
(i) a promoter or a functional variant or fragment thereof which is active in Mycoplasma bacteria, operably linked to a nucleotide sequence encoding a GP35 recombinase, or
(ii) an RNA sequence encoding a GP35 recombinase, and
   2) a second nucleotide arrangement comprising a naturally occurring Mycoplasma sequence, wherein said naturally occurring Mycoplasma sequence is comprised as two non-adjacent nucleotide sequences in the second nucleotide arrangement, each having a minimum length of 5 nucleotides.
Aspect 6. An oligonucleotide modification system comprising:
   1) a first nucleotide arrangement comprising a promoter or a functional variant or fragment thereof which is active in Mycoplasma bacteria, operably linked to a nucleotide sequence encoding a GP35 recombinase, and
   2) a (second) nucleotide arrangement comprising a naturally occurring Mycoplasma sequence, wherein said naturally occurring Mycoplasma sequence is comprised as two non-adjacent nucleotide sequences in the second nucleotide arrangement, each having a minimum length of 5 nucleotides.
Aspect 7. An oligonucleotide modification system comprising:
   1) an RNA sequence encoding a GP35 recombinase, and
   2) a (nucleotide) arrangement comprising a naturally occurring Mycoplasma sequence, wherein said naturally occurring Mycoplasma sequence is comprised as two non-adjacent nucleotide sequences in the second nucleotide arrangement, each having a minimum length of 5 nucleotides.
Aspect 8. An oligonucleotide modification system comprising:
   1) a GP35 recombinase protein, and
   2) a nucleotide arrangement comprising a naturally occurring Mycoplasma sequence, wherein said naturally occurring Mycoplasma sequence is comprised as two non-adjacent nucleotide sequences in the second nucleotide arrangement, each having a minimum length of 5 nucleotides.
Aspect 9. The oligonucleotide modification system according to aspects 1 to 4, or according to aspect 8 wherein the GP35 recombinase protein is recombinant GP35 recombinase protein.
Aspect 10. The oligonucleotide modification system according to aspects 1 to 4, or according to aspect 8 wherein the GP35 recombinase protein is produced by a cell-free protein production method.
Aspect 11. The oligonucleotide modification system according to any one of aspects 4 to 10, wherein the two non-adjacent nucleotide sequences in the (second) nucleotide arrangement that are naturally occurring Mycoplasma sequences are separated from each other by a nucleotide sequence not-naturally occurring in Mycoplasma, preferably the genomic sequence of the Mycoplasma bacterium whose genomic sequence is to be modified.
Aspect 12. The oligonucleotide modification system according to aspect 11, wherein the nucleotide sequence not-naturally occurring in Mycoplasma comprises at least a restriction site, a site-specific recombinase target site or a nucleotide-encoded selection marker or any combination thereof, wherein preferably the site-specific recombinase target site is a lox site.
Aspect 13. The oligonucleotide modification system according to any one of aspects 1 to 12, wherein the GP35 recombinase is a GP35 recombinase having an amino acid sequence which is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the GP35 recombinase.
Aspect 14. The oligonucleotide modification system according to any one of aspects 1 to 13, further comprising a (third) nucleotide arrangement comprising a promoter or a functional variant or fragment thereof which is active in Mycoplasma bacteria, operably linked to a heterologous nucleotide-encoded gene product, preferably wherein the heterologous nucleotide-encoded gene product comprises a therapeutic protein or an immunogenic protein.
Aspect 15. The oligonucleotide modification system according to aspect 14, wherein the heterologous nucleotide-encoded gene product further comprises an exposure signal sequence or secretion signal sequence.
Aspect 16. The oligonucleotide modification system according to any one of aspects 1 to 15, further comprising a (third) nucleotide arrangement comprising a promoter or functional variant or fragment thereof which is active in Mycoplasma bacteria, operably linked to a nucleotide-encoded nuclease or a nucleotide-encoded recombinase.
Aspect 17. The oligonucleotide modification system according to aspect 16, wherein the nucleotide-encoded nuclease comprised in the third nucleotide arrangement is an endonuclease, preferably an endonuclease selected from the group comprising of restriction enzymes, meganucleases, zinc-finger nucleases (ZFNs), transcription-activator like effector nucleases (TALENs), and CRISPR associated (Cas)-based nucleases, more preferably wherein said nucleotide-encoded nuclease is a Cas-based nuclease.
Aspect 18. The oligonucleotide modification system according to aspect 17, wherein the Cas-based nuclease is a type II Cas nuclease, preferably a Cas9 nuclease having a nucleotide sequence encoding a protein which is at least 65% identical, preferably at least 75% identical, more preferably at least 85% identical, most preferably at least 95% identical to the amino acid sequence of Cas9 from Streptococcus pyogenes as defined by SEQ ID NO: 2 based on the total length of the amino acid sequence of the Cas9 or functional variant thereof.
Aspect 19. The oligonucleotide modification system according to aspect 18, further comprising a fourth nucleotide arrangement comprising at least one single guide RNA sequence, or at least one crRNA sequence and a tracrRNA sequence, capable of base pairing with a naturally occurring sequence in a Mycoplasma genome.
Aspect 20. The oligonucleotide modification system according to any one of aspects 1 to 19, wherein at least one nucleotide arrangement comprises a promoter with a nucleotide sequence of at least 65% identity, preferably at least 75% identity, more preferably at least 85% identity, most preferably at least 95% identity to the nucleotide sequence of SEQ ID NO: 3.
Aspect 21. The oligonucleotide modification system according to any one of aspects 1 to 20, wherein at least one of the nucleotide arrangements further comprises a regulatory sequence capable of modulating transcription, preferably wherein the regulatory sequence is a riboswitch.
Aspect 22. Use of a GP35 recombinase for altering the genomic sequence of a Mycoplasma bacterium, preferably a Mycoplasma pneumoniae bacterium.
Aspect 23. The use according to aspect 22, wherein said GP35 recombinase has an amino acid sequence which is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the recombinase.
Aspect 24. A method of altering the genome of a Mycoplasma bacterium, comprising introducing the oligonucleotide modification system according to any one of aspects 1 to 21, or introducing at least one of the nucleotide arrangements as defined in any one of aspects 1 to 21, or a GP35 recombinase protein into a Mycoplasma bacterium.
Aspect 25. The method according to aspect 24, wherein the recombinant GP35 recombinase has an amino acid sequence that is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the GP35 recombinase.
Aspect 26. The method according to aspects 24 or 25, wherein the third arrangement of nucleotides induces cell death in Mycoplasma bacteria not comprising the second arrangement of nucleotides in their genome, preferably wherein the cell death is a direct consequence of DNA breaks in the genome of the Mycoplasma bacterium.
Aspect 27. The method according to any one of aspects 24 to 26, wherein the Mycoplasma is Mycoplasma pneumoniae.
Aspect 28. Use of an oligonucleotide modification system according to any one of aspects 1 to 21, or the method according to any one of aspects 24 to 27 for generating a genomically modified Mycoplasma bacterium.
Aspect 29. The use according to aspect 28, wherein the genomically modified Mycoplasma is an attenuated Mycoplasma strain.

Aspect 30. The use according to aspect 29, wherein the attenuated Mycoplasma bacteria has a reduced toxicity of at least 30%, preferably at least 50%, preferably at least 75%, most preferably at least 85% to a subject, preferably a human subject, compared to a wild type Mycoplasma bacteria, wherein preferably the reduction of toxicity is determined based on the degree of necroptosis and apoptosis in lung epithelial cells, or by assessment of lung lesions, or by assessment of the level of protein markers indicative for an immune response, or by assessment of reduced pulmonary capacity and/or lung volume.
Aspect 31. The use according to any one of aspects 28 to 30, wherein the genetically modified Mycoplasma expresses, and optionally secretes or displays a heterologous protein.
Aspect 32. A Mycoplasma bacterium comprising the oligonucleotide modification system according to any one of aspects 1 to 21, or obtained by the method according to any one of aspects 24 to 27.
Aspect 33. The Mycoplasma bacterium according to aspect 32, wherein at least one of the nucleotide arrangements according to any one of aspects 1 to 21 is integrated in the genomic sequence of the Mycoplasma bacterium.
Aspect 34. The Mycoplasma bacterium according to aspect 33, wherein the at least one nucleotide arrangement integrated in the genomic sequence of the Mycoplasma bacterium comprises a nucleotide sequence encoding GP35 recombinase, preferably a nucleotide sequence encoding GP35 recombinase having an amino acid sequence that is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the GP35 recombinase.
Aspect 35. The Mycoplasma bacterium according to any one of aspects 32 to 34, wherein at least a nucleotide arrangement comprising two non-adjacent naturally occurring Mycoplasma bacterium nucleotide sequences is comprised integrated in the genomic sequence of the Mycoplasma bacterium.
Aspect 36. A kit of parts comprising:
   1) a first nucleotide or amino acid arrangement comprising a promoter or a functional variant or fragment thereof which is active in Mycoplasma bacteria, operably linked to a nucleotide sequence encoding a GP35 recombinase, and/or an RNA sequence encoding said DNA binding protein, and/or GP35 recombinase protein, and
   2) a second nucleotide arrangement comprising a naturally occurring Mycoplasma sequence with a minimum length of 5 nucleotides.
Aspect 37. A kit of parts comprising:
   1) a first nucleotide arrangement comprising a promoter or a functional variant or fragment thereof which is active in Mycoplasma bacteria, operably linked to a nucleotide sequence encoding a GP35 recombinase, or an RNA sequence encoding said GP35 recombinase protein, and
   2) a second nucleotide arrangement comprising a naturally occurring Mycoplasma sequence with a minimum length of 5 nucleotides.
Aspect 38. A kit of parts according to aspects 36 or 37, wherein the first nucleotide arrangement comprises, consists essentially of, or consists of a promoter or a functional variant or fragment thereof which is active in Mycoplasma bacteria, operably linked to a nucleotide sequence encoding a GP35 recombinase.
Aspect 39. A kit of parts according to aspects 36 or 37, wherein the first nucleotide arrangement comprises, consists essentially of, or consists of an RNA sequence encoding a GP35 recombinase.
Aspect 40. A kit of parts comprising:
   1) a GP35 recombinase protein, and
   2) a second nucleotide arrangement comprising a naturally occurring Mycoplasma sequence with a minimum length of 5 nucleotides.
Aspect 41. The kit of parts according to any one of aspects 36 to 40, wherein the nucleotide arrangement comprising the naturally occurring Mycoplasma sequence is comprised as two non-adjacent nucleotide sequences, each having a minimum length of 5 nucleotides Aspect 42. The kit of parts according to any one or aspects 36 to 41, further comprising a third nucleotide arrangement as described in aspects 14 to 18.

### BRIEF DESCRIPTION OF THE DRAWINGS

FIGS. 1A and 1B. Screening of different ssDNA recombinases to perform oligo recombineering in M pneumoniae. (FIG. 1A) Scheme depicting the chromosome of M129MutCm+1 strain, showing the bidirectional replication fork that starts at the origin of replication (ori) and enlarges until reaching the terminus of replication (ter). The plus and minus strands follow the indicated colour code; newly synthesized DNA is either continuous (solid line) or discontinuous way (dashed line). The location of MutCm+1 recombineering at the minus strand of the MPN049 locus is shown, as well as the orientation of CmONsense or CmONantisense editing oligonucleotides. (FIG. 1B) Barplot showing in logarithmic scale the colony-forming units (CFU) obtained for M pneumoniae M129 cells carrying the recombineering sensor (M129MutCm+1 strain) or the recombineering sensor plus a second transposon coding for the different recombinases (M129MutCm+1GP35, M129MutCm+lRecTₛₘ, M129MutCm+1RecTₛₚ, or M129MutCm+1RecT_{sc}). Each group of four bars represents the CFU values obtained for different transformations of the indicated strains, being from left to the to the right (i) mock transformation seed on Cm-selective plates, (ii) CmONantisense editing oligo transformation seed on Cm-selective plates and (iii) CmONsense editing oligo transformation seed on Cm-selective plates. The far left bar of each group represents the mean of CFU counted on non-selective plates for the three transformations done in each strain. The editing rate (edited cells/total cells) obtained with the CmONsense oligonucleotide for each strain is shown above the bars. Those differences in terms of editing rate that were found to be statistically significant (P<0.05) after conducting a paired t-test are indicated with an asterisk (*). Error bars represent the mean of the standard deviation (SEM) of three different biological replicas.

FIG. 2. Western blot analysis of the expression of the different recombinases used in the screening. Bacterial lysates of the indicated Mycoplasma strains were subjected to analysis. All recombinases were immunodetected with anti-FLAG tag mAb. Ribosomal protein RL7 was used as loading control (lower panel) and detected with anti-RL7 pAb.

FIGS. 3A-3C. Optimization of GP35-oligo recombineering protocol for M pneumoniae. (FIGS. 3A-3C) Barplots showing in logarithmic scale the CFU obtained for the M129MutCm+1GP35 strain after transformation with the CmONsense oligonucleotide. For each group of paired bars, the left bars represent the CFU value obtained after seeding the transformation on non-selective plates, whereas the right bars represent the CFU value obtained after seeding the transformation on Cm-selective plates. The editing rate (edited cells/total cells) is shown above each group of bars. Those differences in terms of editing rate that were found to be statistically significant (P<0.05) after conducting a paired t-test are indicated with an asterisk (*). Error bars represent the mean of the standard deviation (SEM) of three different biological replicas. (FIG. 3A) Transformed cells were allowed to recover for different time intervals prior to seeding, as indicated on the x-axis. (FIG. 3B) M129MutCm+1GP35 cells were transformed with different amounts of oligo, as indicated on the x-axis, and seeded on plates at 24-hours after transformation. (FIG. 3C) M129MutCm+1GP35 cells were transformed with 5 μl of the CmONsense oligo, and subjected to 1, 3, 6, or 10 electroporation pulses, as indicated on the x-axis. Cells were allowed to recover for 24 hours before seeding. Cell viability for each condition is expressed as a percentage of that observed after one electroporation pulse, as shown below each bar.

FIGS. 4A-4C. Chromosome locations of the different recombineering sensors. Schemes depict the chromosomes of the M129MutCm+50GP35, M129MutCm+750GP35, and M129MutCm+1800GP35 strains, showing the bidirectional replication fork that would start from the origin of replication (ori) and enlarge until reaching the terminus of replication (ter). The plus and minus strands follow a color code as indicated, as well as the newly synthesized DNA in either continuous (solid line) or discontinuous way (dashed line). Arrows shown at each chromosome indicate the approximate locations of the MutCm+50, MutCm+750, and MutCm+1800 recombineering sensors at the plus strand of the MPN493, MPN582, and MPN034 loci, respectively. The appropriate editing oligonucleotide for each strain is depicted showing their orientation in respect with the chloramphenicol resistance coding gene.

FIGS. 5A and 5B. Efficiency of GP35-oligo recombineering for large chromosomal modifications.

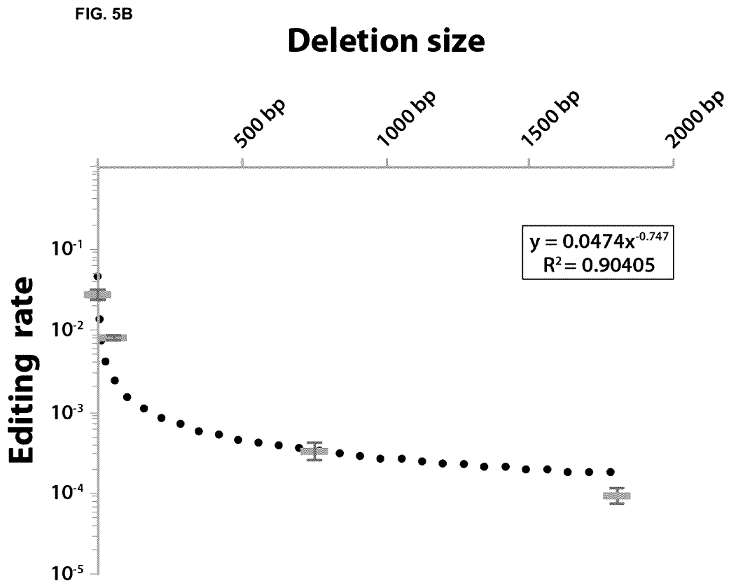

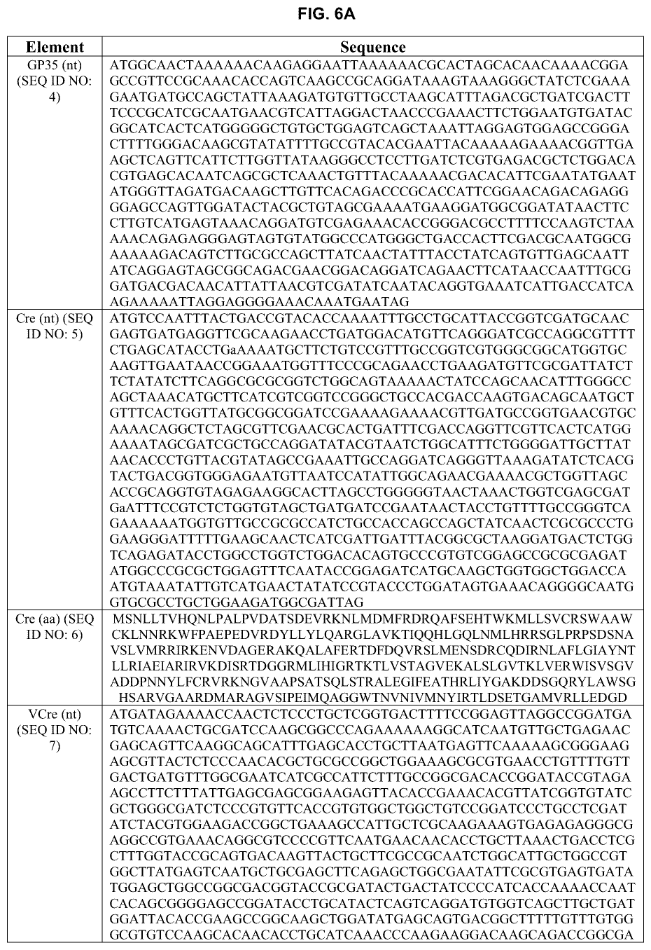

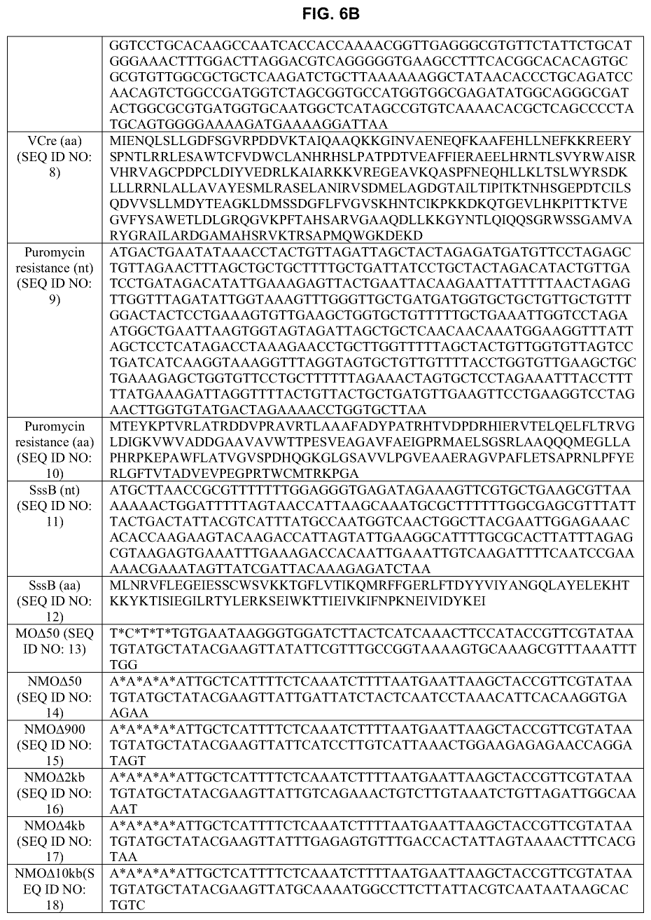

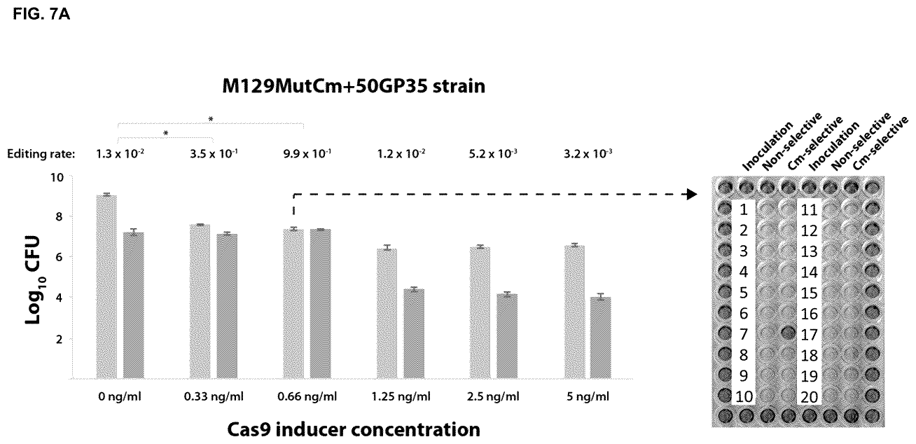

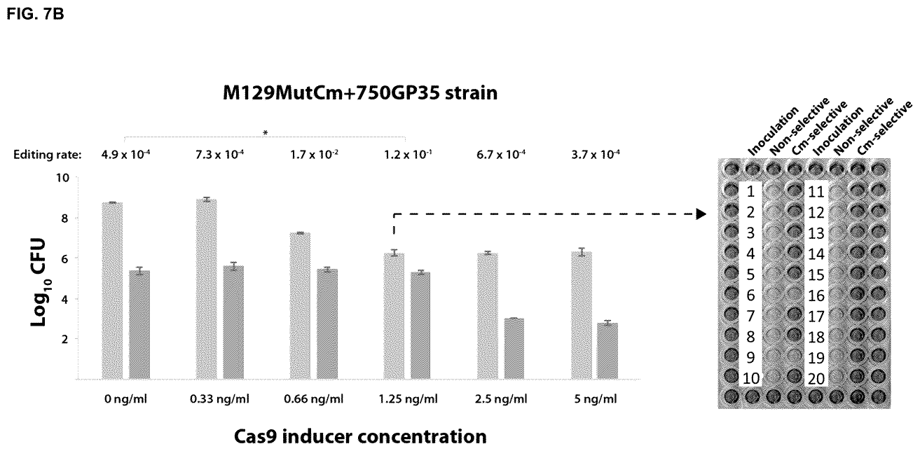

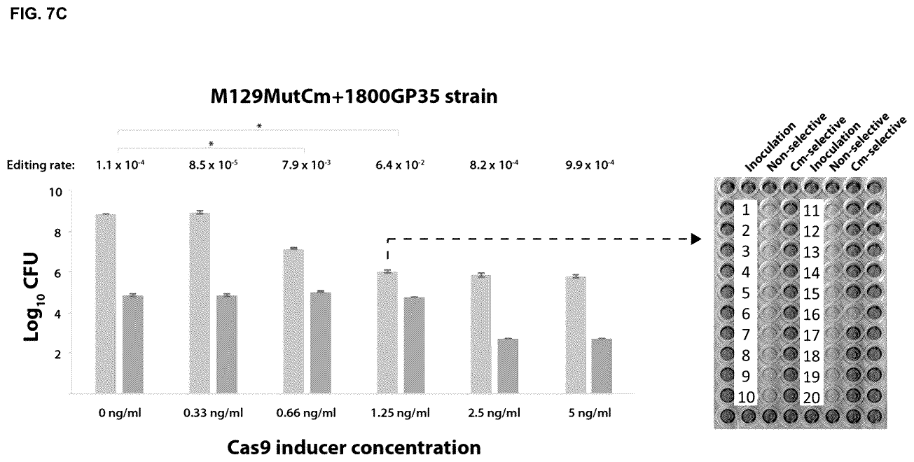

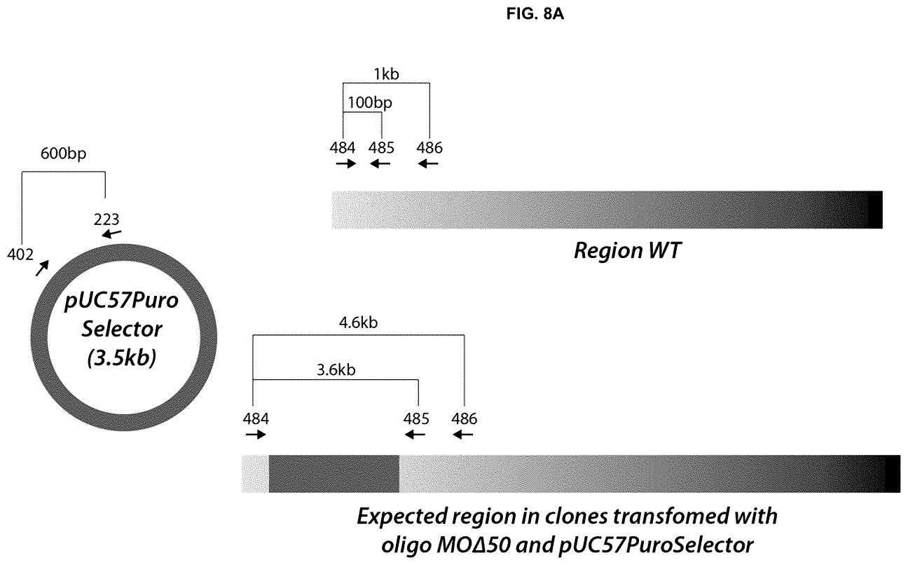

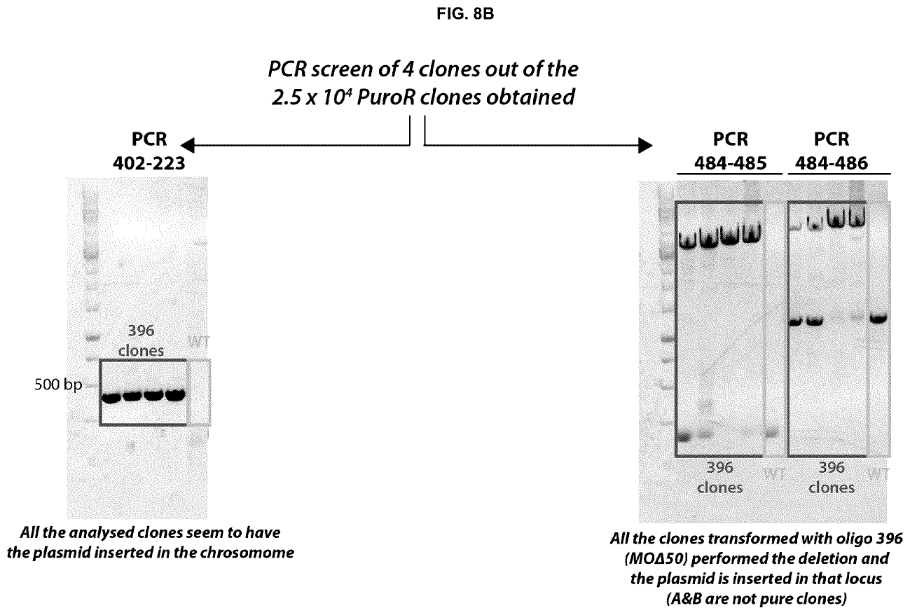

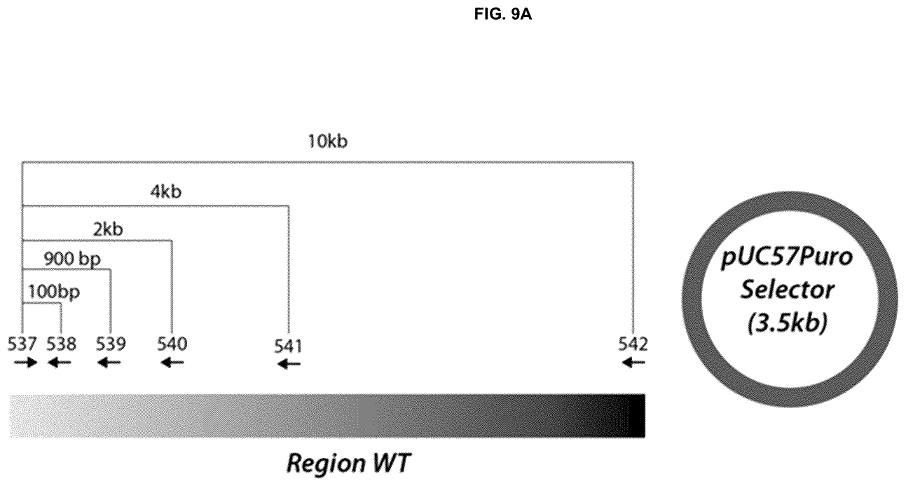

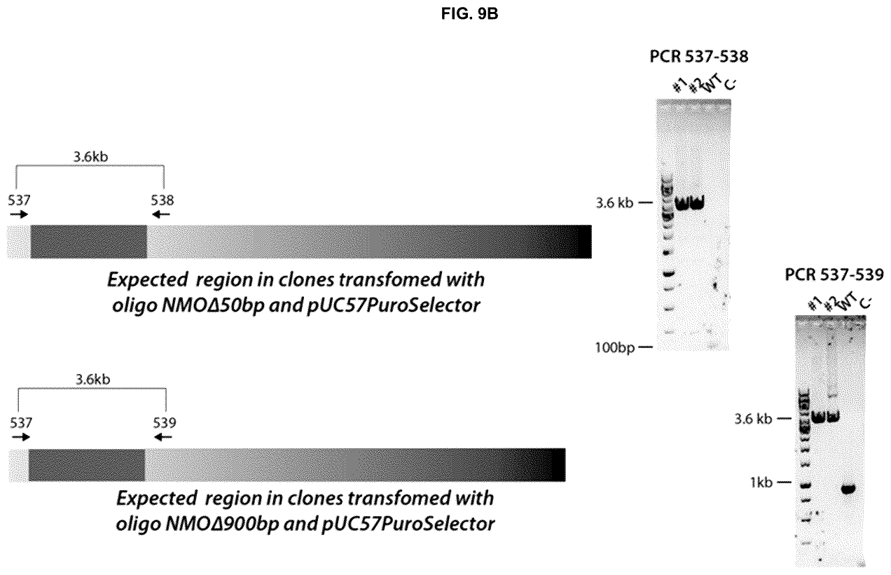

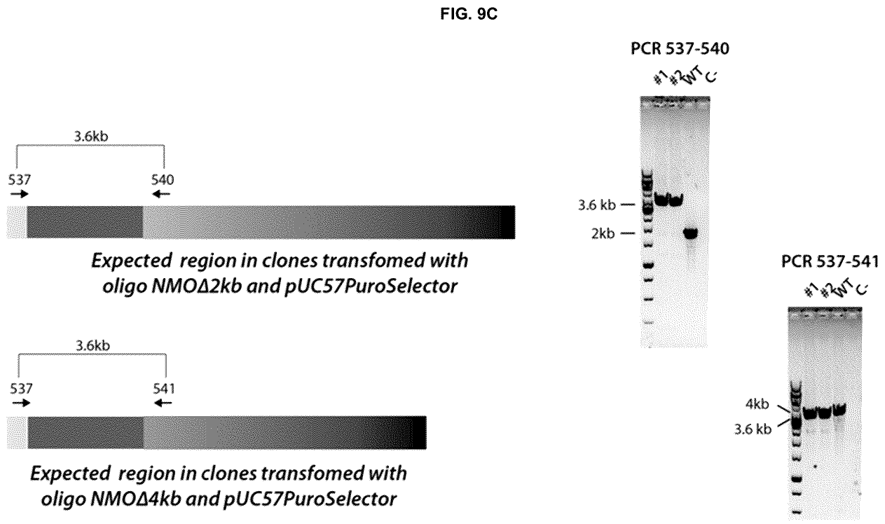

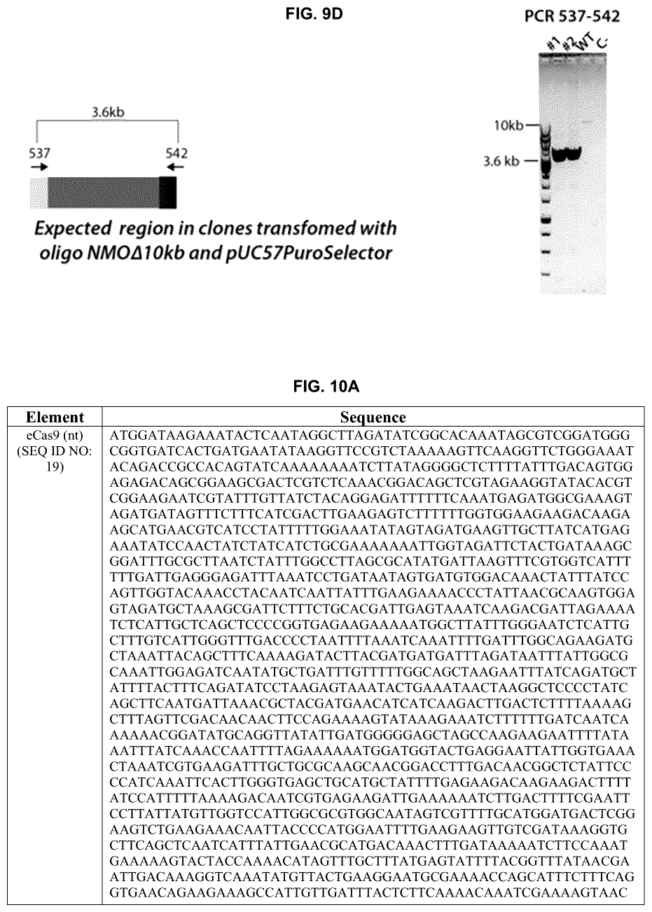

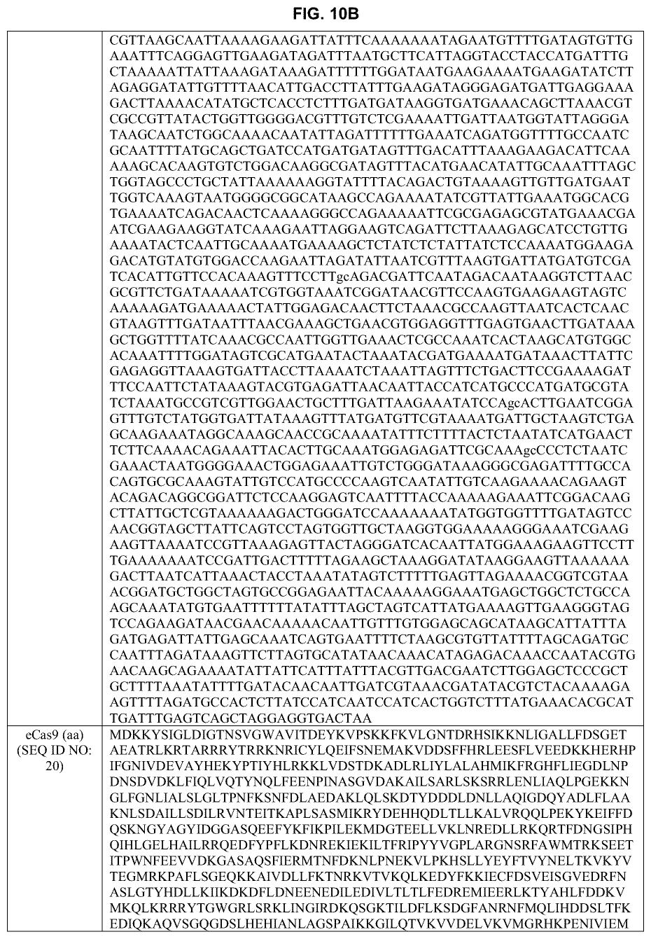

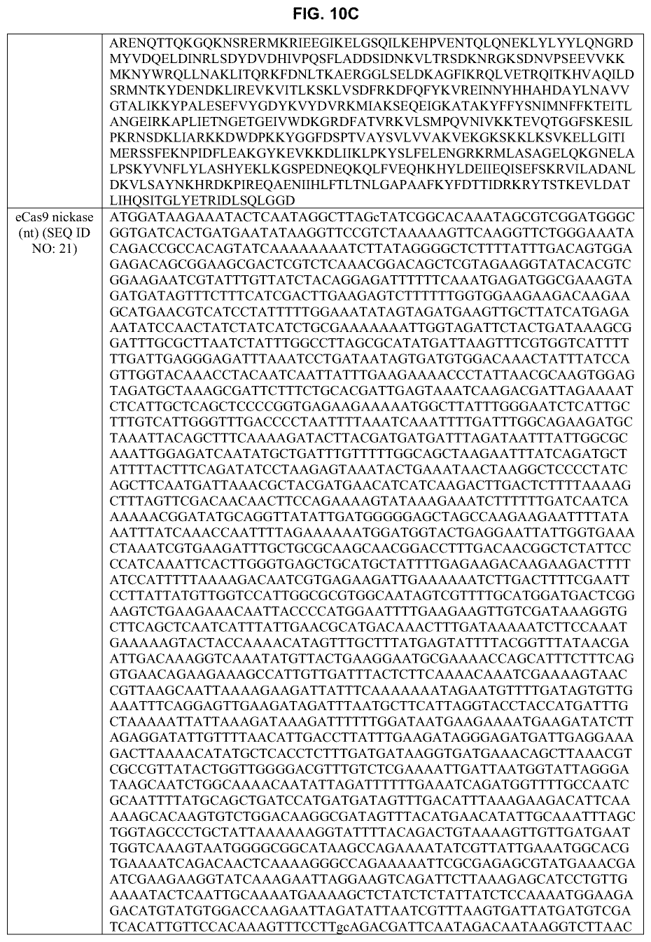

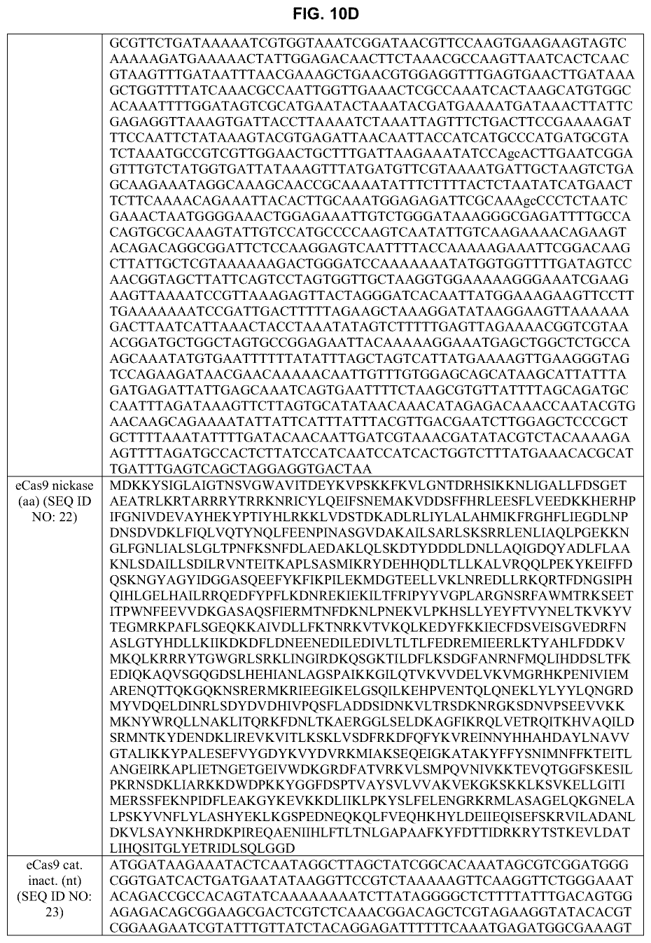

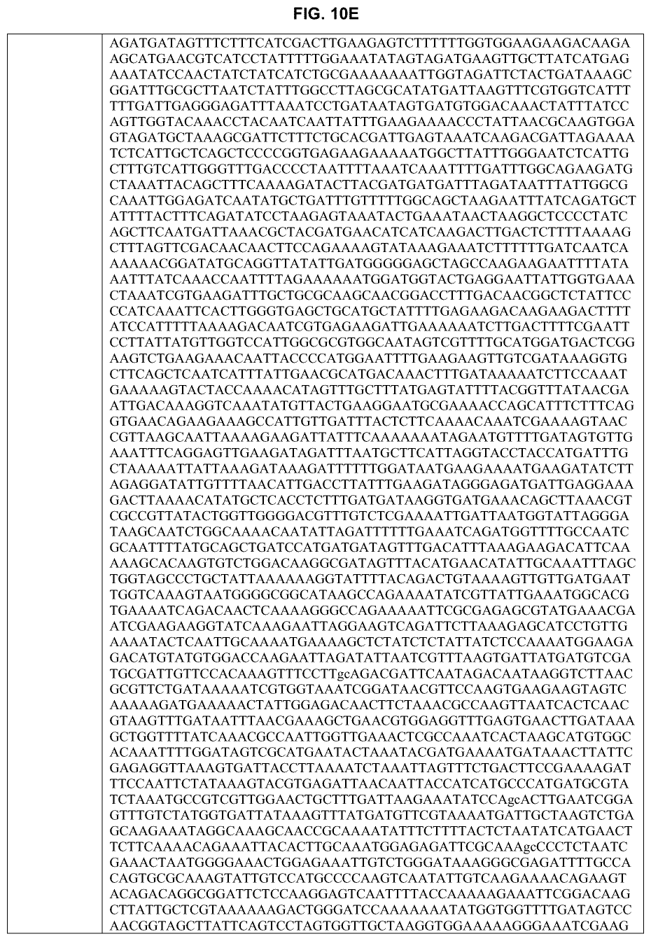

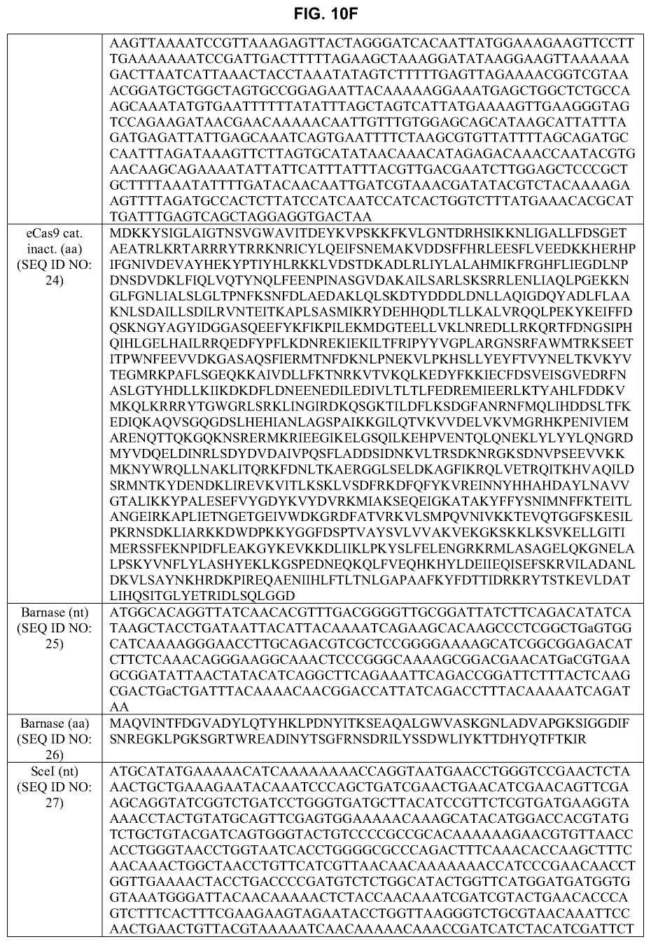

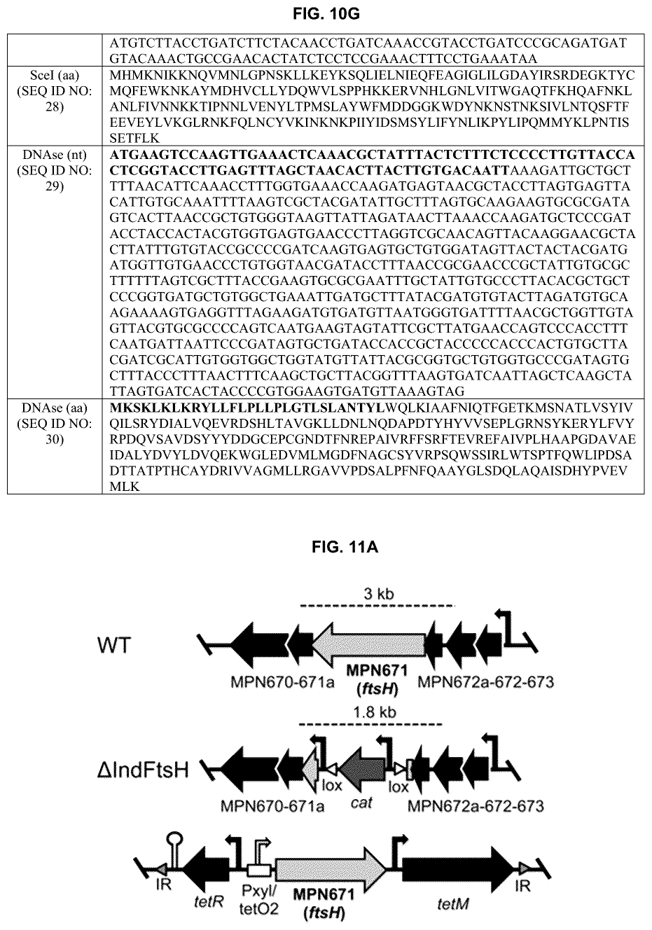

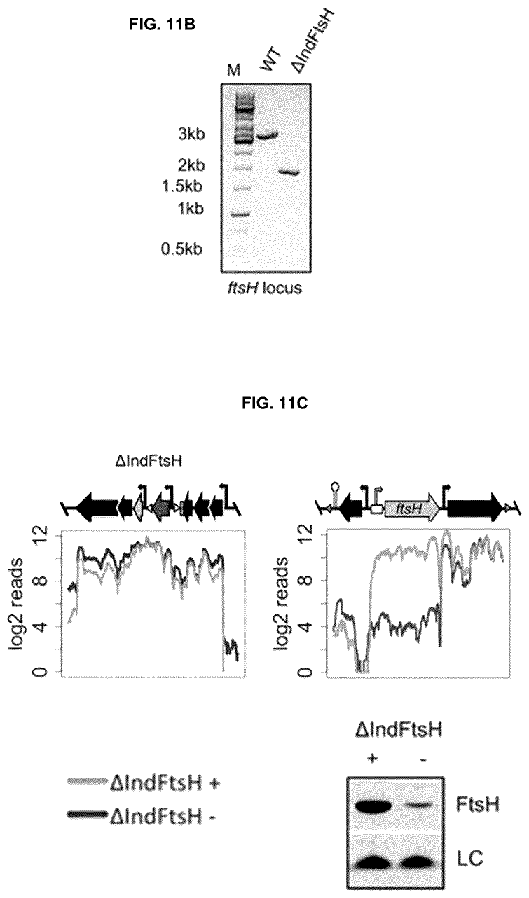

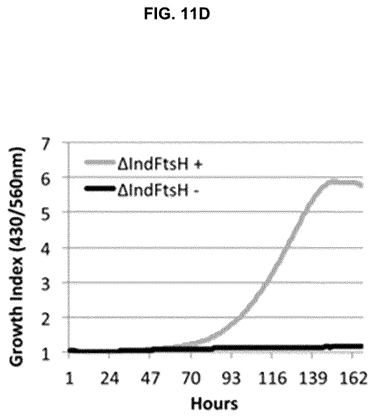

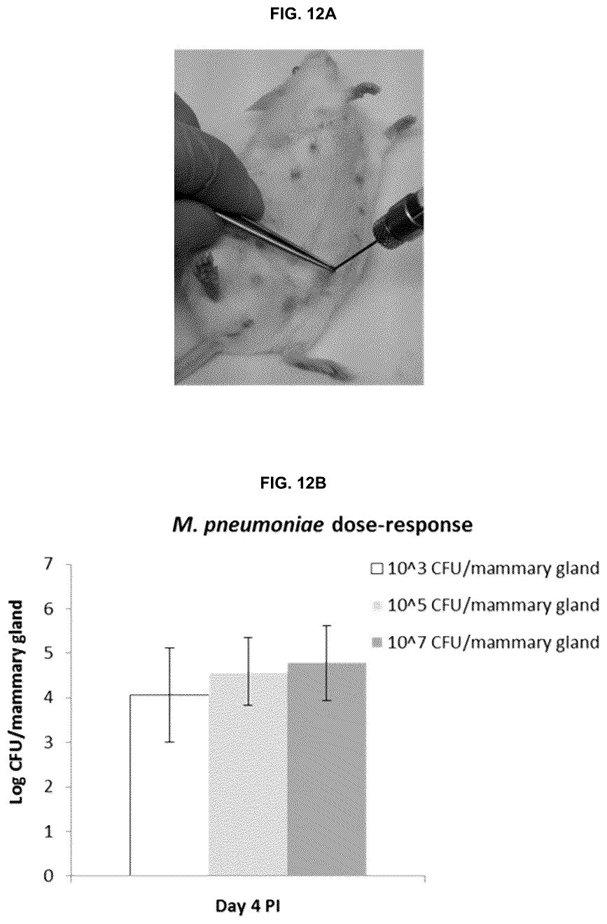

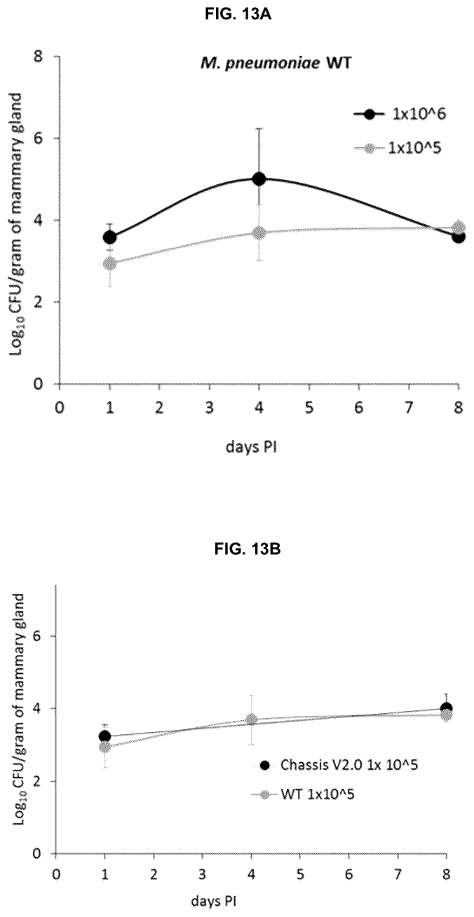

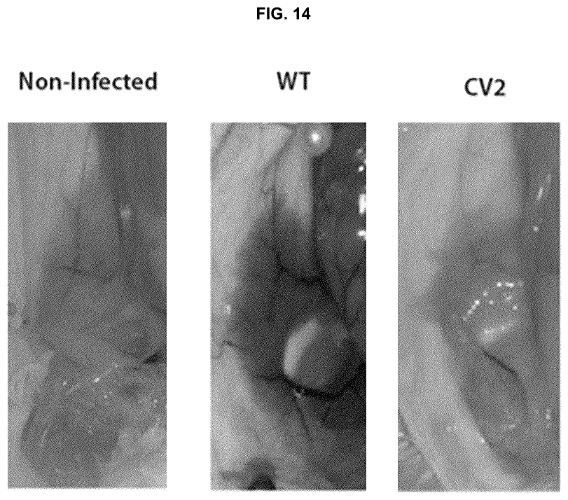

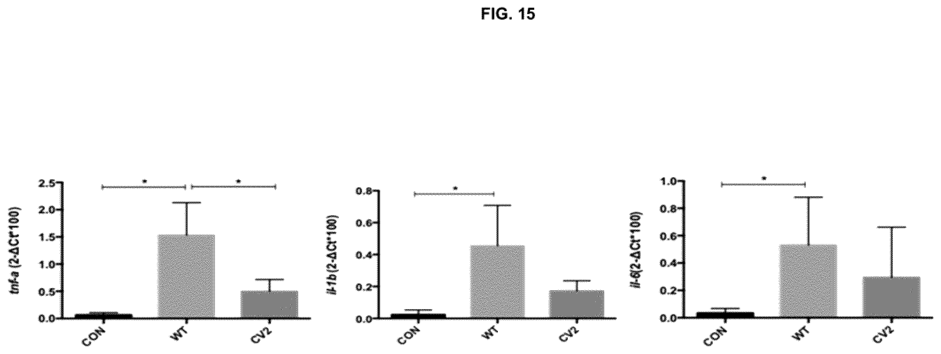

(FIG. 5A) Barplot showing in logarithmic scale the CFU obtained for different recombineering sensor strains (x-axis) after transformation with their respective editing oligos following the conditions established in the optimization screening. For each group of paired bars, the left bars represent the CFU value obtained after seeding the transformation on non-selective plates, whereas the right bars represent the CFU value obtained after seeding the transformation on Cm-selective plates. All strains expressed GP35 recombinase and different recombineering sensors whose activation required the deletion of 50 bp, 750 bp, or 1800 bp, depending on the strain. The editing rate (edited cells/total cells) obtained for each strain is shown on top of each group of bars. The differences in terms of editing rate that were found to be statistically significant (P<0.05) after conducting a paired t-test are indicated with an asterisk (*). Error bars represent the mean of the standard deviation (SEM) of three different biological replicas. (FIG. 5B) Plot comparing the size of the attempted chromosomal deletion and the editing rate obtained for that modification. Each rectangle represents the mean editing rate of three independent biological replicas performed with M129MutCm+1GP35, M129MutCm+50GP35, M129MutCm+750GP35, or M129MutCm+1800GP35 strains. Error bars represent the mean of the standard deviation (SEM) of three different biological replicas. Dotted line represents the decreasing power trend observed between deletion size and efficiency. The equation describing this trend as well as the coefficient of determination (R²) is shown inside the square.

FIGS. 6A and 6B. Relevant sequences to the recombineering approach. Nucleotide (nt) and amino acid (aa) sequences of GP35, Cre, VCre, Puromycin resistance, and SssB elements. Nucleotide sequence of the MOΔ50, NMOΔ50, NMOΔ900, NMOΔ2kb, NMOΔ4kb, and NMOΔ10kb, oligonucleotides. Asterisks shown at the 5′ end of the oligos are indicative for phosphorothioate bonds.

FIGS. 7A-7C. Improvement of editing rates mediated by Cas9-based counterselection. (FIGS. 7A-7C) Barplots showing in logarithmic scale (left side) the CFU obtained for the recombineering sensor strains M129MutCm+50/eiCas9 (FIG. 7A), M129MutCm+750/eiCas9 (FIG. 7B), and M129MutCm+1800/eiCas9 (FIG. 7C). Strains were transformed with their respective editing oligos following the conditions established in the optimization screening. For each group of paired bars, the left bars represent the CFU value obtained after seeding the transformation on non-selective plates, whereas the right bars represent the CFU value obtained after seeding the transformation on Cm-selective plates. All plates were supplemented with different anhydrotetracycline (aTc) concentrations, as indicated on the x-axis. Pictures of the screening plates (right side) of 20 colonies randomly picked from the non-selective plate supplemented with aTc concentrations rendering the highest editing rate possible, as indicated by the dashed arrow. The growth of each clone in non-selective or Cm-selective medium was monitored by the ability ofM pneumoniae cells to acidify and thus mediate a color change (from dark grey when cells have not grown, to light grey when cells proliferated) on the medium containing phenol red. The editing rate (edited cells/total cells) obtained for each strain and condition is shown at the top of each group of bars. Those differences in terms of editing rate that were found to be statistically significant (P<0.05) after conducting a paired t-test are indicated with an asterisk (*). Error bars represent the mean of the standard deviation (SEM) of three different biological replicas.

FIGS. 8A and 8B. Combining ssDNA recombinases (GP35) and site specific recombinases (Cre) to edit Mycoplasma genome at unprecedent efficiencies. On top schemes depicting the structure of pUC57PuroSelector plasmid and the oligos employed to detect its integration into M pneumoniae genome (left), as well as the structure of the M pneumoniae genome and the location of the oligos employed for the screening in those cells that did not perform the intended edition (WT) or those ones that acquired the pursued modification (MOΔ50) (right). At the bottom electrophoresis analysis of the indicated PCR screenings performed in four different clones subjected to modification (396 clones) and one non-treated clone (WT).

FIGS. 9A-9D. Combining ssDNA recombinases (GP35) and site specific recombinases (Cre) to edit Mycoplasma genome at unprecedent efficiencies (II). On the left side schemes depicting the chromosomal structures of the WT strain, and the strains transformed with different editing oligos as indicated, as well as the location of the oligonucleotides employed for the PCR screening. On the right side, electrophoresis analyses of the indicated PCR screening performed in two different clones transformed with the indicated editing oligo, and also on the WT strain.

FIGS. 10A-10G. Sequences of illustrative endonucleases and restrictases suitable for use in the invention. Nucleotide (nt) and amino acid (aa) sequences of enhanced Cas9 (eCas9), eCas9 nickase, catalytically inactive eCas9, Barnase, Scel, and DNAse (MPN142 OPT signal for secretion in bold).

FIGS. 11A-11D. Construction and characterization of FtsH conditional mutants in M pneumoniae. (FIG. 11A). Schematic representation of the genetic architecture of ΔIndFtsH conditional mutant compared to the WT strain. The DNA rearrangement in the ftsH locus and the ftsH inducible platform inserted by transposon delivery are shown. The Pxyl/tetO2 inducible promoter is highlighted with a white bent arrow and the terminator sequence used to isolate the promoter is represented by a hairpin structure. The tetR repressor gene and the resistance markers cat and tetM are also indicated. (FIG. 11B) Agarose electrophoresis gels showing the PCR experiments to confirm the intended genome rearrangements at the ftsH locus. The PCR products and expected sizes for each strain are shown in the scheme of panel A. (FIG. 11C) RNA-seq transcriptional profiles across the modified locus, as well as immunoblots are shown for ΔIndFtsH strain grown under inducing or depleting conditions. Symbols+/−indicate inducing or depleting conditions. LC, represents the loading control. (FIG. 11D) Growth curve analysis for ΔIndFtsH strain grown under inducing (+) or depleting conditions (−) determined by the 430/560 absorbance rate index that reports on pH changes in the medium.

FIGS. 12A and 12B. Study of infection of mice mammary gland with different doses of Mycoplasma WT strain (10\hat{ }3, 10\hat{ }5 and 10\hat{ }7). This experiment was repeated with 10\hat{ }6 and 10\hat{ }5 dose8s and after scarifying the animals at different days (1, 4 and 8 days) with the WT and Chassis strains (FIGS. 9A-9C). The dose of 10\hat{ }5 was determined as the optimal to use this model for the maintenance of Mycoplasma and the time point of for 4 days. We found that both strains behave similarly with the dose of 10\hat{ }5 and that at 4 days is the best time to recover similar CFUs than the one infected. Thus, WT and Chassis strains behave similarly in mammary gland tissue.

FIGS. 13A and 13B. Maintenance of Mycoplasma stains in the mammary gland tissue. (FIG. 13A) Mice infected with WT strain, two doses (10\hat{ }6 and 10\hat{ }5) and sacrificed after 1, 4 and 8 days of infection (5 animals/group). (FIG. 13B) Comparison between WT strain and chassis at different days (1, 4 and 8) after infecting animals with dose of 10\hat{ }5.

FIG. 14. Study of biosafety and maintenance of different chassis strains in vivo. Images of hemorrhagic lesions caused by wild type and different versions of the chassis (CV1 and CV2) strains. The histology of the mast of CV2 is almost identical when compared to non-infected mice.

FIG. 15. Comparison of levels of interleukins in mammary gland tissue after 4 days of infection with WT and Chassis strains (CV2). Left: TNF-α, middle: IL-1β, right: IL-6.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Amino acids are referred to herein with their full name, their three-letter abbreviation or their one letter abbreviation.

“Mycoplasma”, “Mycoplasma bacteria”, or “Mycoplasmas” as used interchangeably herein refers to the mollicute genus Mycoplasma which is characterized by lack of a cell wall around their cell membranes. Therefore, the plasma membrane forms the outer boundary of the Mycoplasma bacterial cell. Due to the absence of a cell wall, Mycoplasma has been found to have versatile shapes ranging from round to oblong, and display pleomorphism. “Pleomorphism” as used herein is a term used in histology and cytopathology to describe cells and/or their nuclei that may contain variable sizes, shape and staining. CulturableMycoplasma species typically form small umbonate colonies on agar. The exact shape of the Mycoplasmas may depend on numerous parameters including osmotic pressure, nutritional quality of the culture medium, and growth phase. Certain Mycoplasma bacteria may be filamentous in their early and exponential growth phases or when attached to surfaces or other cells. The filamentous form may be transitory, and in certain conditions the filaments may branch or fragment into chains of cocci or individual vegetative cells. Alternative species are typically coccoid and do not develop a filamentous phase. Certain species develop specialized attachment tip structures involved in the process of colonization and/or contribute to virulence. Mycoplasma bacteria comprise 16S and 70S type ribosomes and a replicating disc to assist the replication process, and isolation of the genetic material. Mycoplasma bacteria may either live as saprophytes or more commonly as parasites. The term “saprophytes” refers to the chemoheterotropic extracellular digestion that takes place in the processing of decayed organic matter. Mycoplasma bacteria are commonly described as one of the smallest and simplest self-replicating organisms known to date. Naturally occurring Mycoplasma genomes vary from about 500 kilobases (kb) to 1500 kb and GC contents between 23-41 mole percent (mol %) have been described.

Techniques for enrichment and/or isolation of Mycoplasmas from humans, various species of animals, and cell cultures have been extensively described in the art and are well-known to a skilled person (Tully and Razin, Molecular and diagnostic procedures in Mycoplasmology, Vol. 2, 1996). A skilled person is also aware that minimal standards for descriptions of new species have been outlined (Brown et al., Revise standards for description of new species of the class Mollicutes (division Tenericutes), International Journal of Systematic and Evolutionary Microbiology, 2007).

A substantial amount of Mycoplasma species have been described and exemplary Mycoplasma species include those of the following non-exhaustive list: M. adleri, M agalactiae, M agassizii, M alkalescens, M. alligatoris, M. alvi, M. amphoriforme, M anatis, M. anseris, M arginine, M. arthritidis, M auris, M bovigenitalium, M bovirhinis, M. bovis, M bovoculi, M. buccale, M. buteonis, M californicum, M. canadense, M. canis, M capricolum, M capricolum subsp. capricolum, M capricolum subsp. capripneumoniae, M. caviae, M. cavipharyngis, M. ciconiae, M. citelli, M cloacale, M collis, M columbinasale, M. columbinum, M columborale, M. conjunctivae, M corogypsi, M. cottewii, M cricetuli, M. crocodyli, M cynos, M dispar, M edwardii, M elephantis, M. equigenitalium, M equirhinis, M falconis, M fastidiosum, M faucium, M. felifaucium, M feliminutum, M. felis, M. feriruminatoris, M fermentans, M flocculare, M. gallinaceum, M. gallinarum, M gallisepticum, M gallopavonis, M. gateae, M. genitalium, M. glycophilum, M gypis, M haemocanis, M haemofelis, M haemomuris, M hominis, M. hyopharyngis, M hyopneumoniae, M. hyorhinis, M hyosynoviae, M. iguana, M imitans, M indiense, M. iners, M iowae, M lagogenitalium, M leachii, M leonicaptivi, M leopharyngis, M lipofaciens, M. lipophilum, M maculosum, M meleagridis, M microti, M moatsii, M mobile, M molare, M. mucosicanis, M muris, M mustelae, M mycoides, M mycoides subsp. capri, M mycoides subsp. mycoides, M neophronis, M. neurolyticum, M. opalescens, M orale, M ovipneumoniae, M. ovis, M oxoniensis, M. penetrans, M phocicerebrale, M phocidae, M. phocirhinis, M pirum, M pneumoniae, M primatum, M pullorum, M pulmonis, M putrefaciens, M. salivarium, M simbae, M spermatophilum, M spumans, M. sturni, M sualvi, M subdolum, M. suis, M. synoviae, M testudineum, M testudinis, M tullyi, M. verecundum, M. wenyonii, M. yeatsii, M coccoides. When the term Mycoplasma is used herein, this includes the non-limiting list of candidate species Moeniiplasma glomeromycotorum, M aoti, M corallicola, M erythrocervae, M girerdii, M haematoparvum, M haemobos, M. haemocervae, M haemodidelphidis, M haemohominis, M haemolamae, M haemomacaque, M haemomeles, M haemominutum, M haemomuris subsp. musculi, M haemomuris subsp. ratti, M haemovis, M haemozalophi, M kahaneii, M ravipulmonis, M struthiolus, M turicensis, M haemotarandirangiferis, M preputii and others such as M insons, M sphenisci, M vulturis, and M zalophi. Furthermore, it is evident to a skilled person that the term Mycoplasma additionally includes any Mycoplasma strain or species that is generated by genetic or chemical synthesis, or any sort of rational design and/or the reorganization of a naturally occurring Mycoplasma genomic sequence and that the term therefore also covers those Mycoplasma strains and species that are termed “synthetic Mycoplasma”, alternatively “Mycoplasma laboratorium”, “Mycoplasma synthia”, or even short “Synthia” in the art (Gibson et al., Creation of a bacterial cell controlled by a chemically synthesized genome, Science, 2010). Hence, in certain embodiments described throughout this specification, the Mycoplasma species subject of the invention have as genomic sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to a naturally occurring Mycoplasma bacterium. In certain embodiments, the Mycoplasma bacterium is M pneumoniae M129-B7 as available from the American Type Culture Collection accession number 29342.

Accordingly, a first aspect of the invention is directed to an oligonucleotide modification system comprising a DNA binding protein or a first nucleotide arrangement which comprises a promoter or a functional variant or fragment thereof which is active in Mycoplasma bacteria, operably linked to a nucleotide sequence encoding said DNA binding protein, or an RNA sequence encoding said DNA binding protein, and a second nucleotide arrangement comprising a naturally occurring Mycoplasma sequence with a minimum length of 5 nucleotides.

In certain embodiments, the oligonucleotide modification system is a set of nucleotide arrangements comprising a first nucleotide arrangement which comprises a promoter or a functional variant or fragment thereof which is active in Mycoplasma bacteria, operably linked to a nucleotide-encoded DNA binding protein and a second nucleotide arrangement comprising a naturally occurring Mycoplasma sequence with a minimum length of 5 nucleotides. In alternative embodiments, the oligonucleotide modification system comprises a DNA binding protein and a second nucleotide arrangement comprising a naturally occurring Mycoplasma sequence with a minimum length of 5 nucleotides. In alternative embodiments, the oligonucleotide modification system comprises an RNA sequence encoding a DNA binding protein and a second nucleotide arrangement comprising a naturally occurring Mycoplasma sequence with a minimum length of 5 nucleotides.

The term “oligonucleotide modification system” as used in the context of the invention refers to a collection, i.e. a multitude of distinct molecular elements, capable of specific targeting and subsequently modifying an nucleotide sequence. Oligonucleotide modification systems have been described in the art, and can be introduced in a target cell under various forms such as described further herein, including DNA, RNA, proteins, or any combination hereof. A skilled person understands that in the context used herein, the term modification system is indicative for a genome engineering system, thus a collection of distinct molecular elements that attribute to a change in sequence of a targeted nucleotide sequence. The term “oligonucleotide modification system” does by no means indicate any limitation on the physical entities comprised in the modification system, and only reflects the intended entity to be modified. Hence, the oligonucleotide system may comprise one or more components that do not fall under the term “oligonucleotide”, but for example would be appreciated by a skilled person as e.g. proteins. For example, the GP35 recombinase may be present in the modification system as a oligonucleotide encoding said recombinase, or as a GP35 recombinase protein as such. While modification of the genomic sequence of a target cell is envisaged, the term is not limited to this interpretation and therefore instances wherein oligonucleotides comprised in a cellular system not part of the genomic sequence of said cell are modified by the system are also envisaged by the term. Unless explicitly mentioned otherwise, the intended nucleotide sequence targeted by the modification system is a DNA nucleotide sequence.

“DNA binding protein” as used herein is indicative for proteins that comprise a DNA binding domain or at least are capable to bind DNA. DNA binding proteins are thus commonly described as proteins that have a specific or at least general affinity for single or double stranded DNA. DNA binding proteins can either bind to the major groove of DNA, the minor groove, or both. Non-limiting examples of DNA binding proteins are transcription factors, polymerases, (designer) nucleases, and histones Unless indicated otherwise, DNA binding proteins that are able to direct inclusion or depletion of DNA sequences at defined nucleotide sequences, preferably an nucleotide sequence comprised in a genomic sequence. A skilled person is aware that besides DNA binding proteins, other DNA binding molecules may be envisaged such as but not limited to DNAzymes (Morrison et al., DNAzymes: Selected for applications, Small Methods, 2018).

“Nucleotide arrangements” as used herein, or synonymously “nucleotide sequences”, “polynucleotide arrangements”, “polynucleotide sequences”, refers to a sequence of a multitude of nucleotides physically connected to form a nucleotide sequence. Unless the contrary is mentioned, the nucleotide arrangements are not presented as part of, or embedded in their naturally occurring genome. Means and methods to obtain, generate and modify isolated polynucleotide sequences are well known to a person skilled in the art (Alberts et al., Molecular Biology of the Cell. 4th edition, 2002). In certain embodiments, the nucleotide arrangement is a double stranded DNA sequence. In alternative embodiments, the nucleotide arrangement is a single stranded DNA sequence. In yet alternative embodiments, the nucleotide arrangement is a single stranded RNA sequence. In yet alternative embodiments, the nucleotide arrangement is a double stranded RNA sequence. In even alternative embodiments, the nucleotide arrangement is a DNA/RNA hybrid oligonucleotide.

In certain embodiments, the second nucleotide arrangement consists exclusively of naturally occurring nucleotide sequences. In further embodiments, the second nucleotide arrangement consists exclusively of naturally occurring nucleotide sequences that are occurring in said nucleotide arrangement in an order different of their order in the genomic sequence of Mycoplasma (i.e. in a scrambled order). In alternative embodiments, the nucleotide arrangements comprise non-naturally occurring nucleotides between the naturally occurring sequences.

In certain embodiments, the oligonucleotide modification system comprises multiple DNA sequences (i.e. arrangements or a set of nucleotide arrangements). In further embodiments, the set of nucleotide arrangements may comprise multiple RNA sequences (i.e. arrangements). In alternative further embodiments, the set of nucleotide arrangements may comprise both DNA nucleotide arrangements and RNA nucleotide arrangements. In certain embodiments, the nucleotide arrangement is a recombinant arrangement. In certain embodiments, the (gene product of the) first nucleotide arrangement is a single stranded DNA binding protein, preferably a recombinase, more preferably a GP35 recombinase, even more preferably a SPP1 GP35 recombinase as defined further herein. Alternatively, the nucleotide sequence encoding the DNA binding protein, preferably the GP35 recombinase as described herein is replaced by GP35 recombinant protein as such or a functional fragment thereof. In these embodiments, the term “first nucleotide arrangement” may be exchanged for the more appropriate term “amino acid arrangement”, which in turn may be interchangeably used with the term “amino acid sequence”. Combinations of oligonucleotide modification systems comprising both GP35 recombinase encoded in a nucleotide arrangement and GP35 recombinase protein are also envisaged.

By means of guidance and not limitation, any nucleotide arrangement can be part of an expression vector such as a plasmid optionally a non-replicative plasmid, a phagemid, a bacteriophage, a bacteriophage-derived vector, an artificial chromosome, a minicircle, a lentiviral vector, a retroviral vector, an adenoviral or adeno-associated viral vector, a piggyback vector, or a tol2 vector. A skilled person is aware of these different types of constructs and their generation and manipulation has been detailed at numerous instances (Sambrook et al., Molecular cloning: a laboratory manual, ISBN 0879693096, 1989 and the corresponding updated 4^{th} Edition, Cold Spring Harbor Laboratory Press, 2012). Furthermore, it is evident to a skilled person that plasmid DNA, or (circular) recombinant DNA is commonly referred to in the art as copy DNA, complement DNA, or even referred to by the abbreviation “cDNA”, which may each be used interchangeably. In certain embodiments, a nucleotide arrangement comprised in the oligonucleotide modification system is part of a bicistronic expression construct. In further embodiments, the nucleotide arrangement is incorporated, i.e. inserted, in a cellular genome, preferably a genomic sequence of a Mycoplasma bacterium, more preferably the genomic sequence of the Mycoplasma bacterium whose genomic sequence is to be modified. In yet further embodiments, the nucleotide arrangement is part of a cellular genome, e.g. a de novo designed cellular genome or a mutagenized or synthetic Mycoplasma bacterium. In further embodiments, the nucleotide arrangement is comprised in a bacterial artificial chromosome or a yeast artificial chromosome. In certain embodiments, one or more of the nucleotide arrangements described herein are comprised in a genomically modified Mycoplasma strain having as reference genome the genome of M. pneumoniae M129-B7 as available from the American Type Culture Collection accession number 29342. In certain embodiments, the 5′ and/or 3′ end of the polynucleotide arrangement is modified to improve the stability of the sequence in order to actively avoid degradation. Suitable modifications in this context include but are not limited to biotinylated nucleotides and phosphorothioate nucleotides. In certain embodiments, the polynucleotide sequence, multiple nucleotide arrangements, or the complete oligonucleotide modification system as disclosed herein is comprised in a bacteriophage. In further embodiments, the polynucleotide sequence, multiple nucleotide arrangements, or the complete oligonucleotide modification system as disclosed herein is comprised in a bacteriophage in the form of a gene drive. A skilled person is aware of the term “gene drive” as it is further described in the present disclosure. The term “bacteriophage” as described herein is indicative for a virus that infects and optionally is able to replicate within bacteria and archaea, which may be modified for therapeutic purposes as has been described in the art (e.g. Principi et al., Advantages and Limitations of Bacteriophages for the Treatment of Bacterial Infections, Frontiers in Pharmacology, 2019).

The term “promoter” as defined herein is a region of DNA that initiates transcription of a particular gene and hence enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene. Promoters may be located in close proximity of the start codon of genes, in preferred embodiments on the same strand and typically upstream (5′) of the gene. Promoters may vary in size, and are preferably from about 100 to 1000 nucleotides long. In certain embodiments, the promoter may be a constitutive promoter. A constitutive promoter is understood by a skilled person to be a promoter whose expression is constant under the standard culturing conditions, i.e. a promoter which expresses a gene product at a constant expression level. In alternative embodiments, the promoter may be an inducible (conditional) promoter. It is understood that inducible promoters are promoters which are responsive at least one induction cue. Inducible promoters, and more specifically bacterial inducible promoter systems have been described in great detail in the art (inter alia in Brautaset et al., Positively regulated bacterial expression systems, Microbial biotechnology, 2009). In certain embodiments, the inducible promoter is chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be regulated by other transcription factors that are constitutive or are themselves directly regulated by chemical or physical cues. In certain embodiments, the promoter is a TetR promoter part of a Tet-On or Tet-off system (Krueger et al., Tetracycline derivatives: alternative effectors for Tet transregulators, Biotechniques, 2004, and, Loew et al., Improved Tet-responsive promoters with minimized background expression, BioMedCentral Biotechnology, 2010). In further embodiments, the concatenation of different sequence elements may be considered as an operon. In certain embodiments, different nucleotide arrangements as described herein may comprise different promoter sequences.

“Control sequences” or “regulatory sequences” as used interchangeably herein refer to any nucleotide sequence which is capable of increasing or decreasing the expression of specific genes. This regulation may be imposed by either influencing transcription rates, translation rates, or by modification of the stability of the sequence. In further embodiments, the polynucleotide sequence comprises regulatory elements such as but not limited to the following: enhancers, selection markers, origins of replication, linker sequences, polyA sequences, terminator sequence, and degradation sequences. In certain embodiments, at least one nucleotide arrangement comprises one or more suitable control sequences. In certain embodiments, the control sequences are identical for all nucleotide arrangements. In alternative embodiments, different control sequences are used for or within different nucleotide arrangements. In certain embodiments, the control sequences are control sequences naturally occurring in Mycoplasma bacteria. In other embodiments, the control sequences are adapted to perform their intended function in Mycoplasma bacteria. It is evident to the skilled person that any component of the oligonucleotide modification system as described herein may further comprise tag sequences that ameliorate purification or localization. Both oligonucleotide motifs and sequences that bind to other oligonucleotides or proteins and amino acid motifs or sequences are envisaged. Non-limiting examples of amino acid tag sequences and linker sequences are described further below.

“Operon” as used herein refers to a functional unit of DNA containing a cluster of genes in which all genes are controlled by a single promotor. It is evident to a skilled person that genes from an operon are co-transcribed. Transcribed genes from an operon are transcribed to a single mRNA strand and may be either translated together in the cytoplasm or spliced to generate monocistronic mRNAs that may be translated separately.

“Origin of replication” also known as “ORI” refers to a sequence at which replication is initiated in either prokaryotic or eukaryotic organisms. DNA replication may proceed from this point bidirectionally or unidirectionally. Commonly used prokaryotic origins or replication include but are by no means limited to pMB1, modified pMB1, pBR322, ColEl, ColEl derivative, F1, R6K, p15A, pSC101, and pUC.

The wording “operably linked” refers to a multitude of genetic elements that are joined as part of the same nucleic acid molecule, suitably positioned and oriented for transcription to be initiated from the promoter. DNA operably linked to a promoter is under transcriptional initiation regulation of the promoter or in functional combination therewith.

The terms “plasmid vector”, “expression vector” or “vector” as used herein refers to nucleic acid molecules, typically DNA, to which nucleic acid fragments, preferably the recombinant nucleic acid molecule as defined herein, may be inserted and cloned, i.e., propagated. Hence, a vector will typically contain one or more unique restriction sites, and may be capable of autonomous replication in a defined cell or vehicle organism such that the cloned sequence is reproducible. A vector may also preferably contain a selection marker, such as, e.g., an antibiotic resistance gene, to allow selection of recipient cells that contain the vector.

In certain embodiments, multiple recombination systems and/or short target sequence sites may be used in a larger recombination scheme within a cell. In certain embodiments, different short target sequences may display varying degrees of cross-reactivity in terms of recombination potential. In alternative embodiments, there is no cross-reactivity between different target sequences simultaneously present within one cell.

In certain embodiments, the naturally occurring Mycoplasma sequence comprised in the second nucleotide arrangement has a minimum length of at least 6, at least 7, at least 8, at least 9, at least 10, at least 15, at least 20, at least 25, at least 30, at least 35, at least 40 nucleotides. In certain embodiments, the naturally occurring Mycoplasma sequence comprised in the second nucleotide arrangement has a length of between 5 and 15 nucleotides, of between 5 and 20 nucleotides, of between 5 and 25 nucleotides, of between 5 and 30 nucleotides, of between 5 and 35 nucleotides, of between 5 and 40 nucleotides, of between 5 and 50 nucleotides, of between 5 and 75 nucleotides, of between 5 and 100 nucleotides, of between 5 and 150 nucleotides, of between 5 and 250 nucleotides, of between 5 and 500 nucleotides, of between 5 and 1000 nucleotides, of between 5 and 2000 nucleotides, of between 5 and 5000 nucleotides. In certain embodiments, the naturally occurring mycoplasma sequence comprised in the second nucleotide arrangement has a length of between 10 and 100 nucleotides, between 20 and 200 nucleotides, between 50 and 500 nucleotides, between 75 and 750 nucleotides.

In certain embodiments, the naturally occurring Mycoplasma sequence is comprised as two non-adjacent nucleotide sequences in the second nucleotide arrangement, each having a minimum length of 5 nucleotides. It is evident to a skilled person that the term “adjacent” may be interchangeably used in this context for “immediately preceding”, “neighbouring” or “immediately following”, and that “non-adjacent” accordingly means that two elements, such as two nucleotide sequences in the context of the invention, are “not immediately preceding or following” the other.

In certain embodiments, the two non-adjacent nucleotide sequences of naturally occurring Mycoplasma sequences each have a length of between 5 and 1000 nucleotides, between 7 and 1000 nucleotides, between 9 and 1000 nucleotides, between 10 and 1000 nucleotides, between 11 and 1000 nucleotides, between 12 and 1000 nucleotides, between 13 and 1000 nucleotides, between 14 and 1000 nucleotides, between 15 and 1000 nucleotides, preferably between 15 and 500 nucleotides, between 15 and 300 nucleotides, between 15 and 250 nucleotides, between 15 and 200 nucleotides, between 15 and 150 nucleotides, between 15 and 150 nucleotides, between 15 and 100 nucleotides, between 15 and 50 nucleotides. In certain embodiments the two non-adjacent nucleotide sequences in the second nucleotide arrangement of naturally occurring Mycoplasma sequences each have a length of between 20 and 60 nucleotides, preferably a length of between 30 and 50 nucleotides, more preferably a length of between 40 and 50 nucleotides. In certain embodiments, the two non-adjacent nucleotide sequences in the second nucleotide arrangement of naturally occurring Mycoplasma sequences each have a minimum length of 5 nucleotides, a minimum length of 7 nucleotides, a minimum length of 9 nucleotides, a minimum length of 11 nucleotides, a minimum length of 15 nucleotides, a minimum length of 20 nucleotides, a minimum length of 25 nucleotides, a minimum length of 30 nucleotides, a minimum length of 35 nucleotides, preferably a minimum length of 40 nucleotides. In certain embodiments, the length of the non-adjacent naturally occurring Mycoplasma sequences in the second nucleotide arrangement is identical. In alternative embodiments, the length of the non-adjacent naturally occurring Mycoplasma sequences in the second nucleotide arrangement is different. In certain embodiments, the distance between the two non-adjacent nucleotide sequences of the naturally occurring Mycoplasma sequences in the second nucleotide arrangement is at least 1, at least 2, at least 5, at least 9, at least 10, at least 20, at least 50, at least 100, at least 200, at least 500 bases.

In alternative embodiments, wherein the (second) nucleotide arrangement comprises a single (i.e. uninterrupted or continuous) sequence that is naturally occurring in a Mycoplasma bacterium whose genomic sequence is to be modified, said sequence may have a length of from 5 to 10000 nucleotides, preferably from 15 to 7500 nucleotides, preferably from 30 to 7500 nucleotides, preferably from 35 to 7500 nucleotides, more preferably from 50 to 5000 nucleotides, even more preferably from 50 to 2500 nucleotides, even more preferably from 50 to 1000 nucleotides, yet even more preferably from 50 to 750 nucleotides, yet even more preferably from 50 to 500 nucleotides, yet even more preferably between 100 to 500 nucleotides. In alternative embodiments, the nucleotide arrangement comprises a single sequence that is naturally occurring in Mycoplasma or a walled relative of Mycoplasma (e.g. NCBI taxonomy ID: 31969). By means of illustration and not limitation, the walled Mycoplasma relative may be Mycoplasma phage phiMFV1 (NCBI taxonomy ID: 280702), Mycoplasma phage MAV1 (NCBI taxonomy ID: 75590), or Mycoplasma phage P1 (NCBI taxonomy ID: 35238). In yet alternative embodiments, the nucleotide arrangement comprises one or more sequences that are naturally occurring in an organism selected from the group consisting of: Mycoplasma, Mycoplasma phage phiMFV1 (NCBI taxonomy ID: 280702), Mycoplasma phage MAV1 (NCBI taxonomy ID: 75590), and Mycoplasma phage P1 (NCBI taxonomy ID: 35238). A skilled person readily appreciates that the oligonucleotide arrangement may comprise distinct sequences which each naturally occur in distinct organisms.

In certain embodiments, the two non-adjacent nucleotide sequences of naturally occurring Mycoplasma sequences in the second nucleotide are separated from each other by three nucleotides which are not directly linking said two nucleotide sequences in a naturally occurring Mycoplasma genomic sequence. In certain embodiments, the three nucleotides encode a different amino acid than the one occurring at the corresponding position in a naturally occurring Mycoplasma genomic sequence. In alternative embodiments, the three nucleotides form a stop codon. In yet alternative embodiments, the three nucleotides encode the same amino acid as the one occurring at the corresponding position in a naturally occurring Mycoplasma genomic sequence. In a certain embodiment, the two non-adjacent nucleotide sequences of naturally occurring mycoplasma sequences in the second nucleotide arrangement are separated from each other by one or two nucleotides. In alternative embodiments, the two-non-adjacent nucleotide sequences of naturally occurring Mycoplasma sequences in the second nucleotide are separated from each other by more than 3 nucleotides, preferably more than 4, more than 5, more than 10, more than 20, more than 50, more than 100, more than 200, more than 500, or even more than 1000 nucleotides which are not directly linking said two nucleotide sequences in a naturally occurring Mycoplasma genomic sequence. In certain embodiments, each distinct nucleotide sequences comprised in the second oligonucleotide arrangement is an nucleotide sequence occurring in Mycoplasma albeit occurring in Mycoplasma in different arrangements or different genomic locations.

In certain embodiments, the naturally occurring Mycoplasma sequence or the two non-adjacent naturally occurring Mycoplasma sequences of naturally occurring Mycoplasma sequences in the second nucleotide arrangement are separated from each other by a nucleotide sequence not occurring in Mycoplasma. In further embodiments, the nucleotide sequence not occurring in Mycoplasma is a codon-optimized Mycoplasma sequence. By “non-adjacent”, it is evident to a skilled person that the two naturally occurring Mycoplasma sequences are not located immediately adjacent to each other in the second nucleotide arrangement when considered either in a 5′ to 3′ direction, or 3′ to 5′ direction. In certain embodiments, the nucleotide sequence not occurring in Mycoplasma may be a scrambled or shuffled sequence or a complementary sequence of a naturally occurring Mycoplasma sequence. In certain embodiments, the nucleotide sequence not naturally occurring in Mycoplasma has a length of 1 nucleotide, preferably between 1 and 5 nucleotides, between 1 and 10 nucleotides, between 1 and 34 nucleotides, between 1 and 48 nucleotides, between 1 and 100 nucleotides, between 1 and 200 nucleotides, between 1 and 500 nucleotides, between 1 and 1000 nucleotides, between 1 and 2500 nucleotides, between 1 and 5000 nucleotides, between 1 and 10000 nucleotides. In certain embodiments, the nucleotide sequence not naturally occurring in Mycoplasma has a length of between 10 and 50 nucleotides, between 15 and 50 nucleotides. In certain embodiments the nucleotide sequence not naturally occurring in Mycoplasma has a length of at least 3 nucleotides, at least 5 nucleotides, at least 10 nucleotides, at least 15 nucleotides, at least 25 nucleotides, at least 30 nucleotides, at least 35 nucleotides, at least 50 nucleotides, at least 100 nucleotides, at least 250 nucleotides, at least 500 nucleotides, at least 1000 nucleotides, at least 2500 nucleotides, at least 5000 nucleotides, at least 10000 nucleotides.

The term “naturally occurring” when referring to a nucleotide sequence is indicative that said nucleotide sequence is identical to a (fragment of a) nucleotide sequence of a specific genome of a wild type or mutant organism (here, Mycoplasma). Likewise, the term “non-naturally occurring” refers to a nucleotide sequence not occurring in a genomic sequence of wild type or unmodified Mycoplasma, i.e. in a wild type or mutant organism naturally occurring in nature. Non-naturally is also indicative for any sequence that is not present in the targeted Mycoplasma strain, but may be present in the genomic sequence of any distinct Mycoplasma strain or any other organism. Hence, a non-naturally sequence may be obtained from any other Mycoplasma or even organism that is not the Mycoplasma subject to the oligonucleotide modification system. Furthermore, a non-naturally occurring sequence may be obtained from, or derived from, a naturally occurring sequence by introducing modifications including the changing of nucleotides, deleting of nucleotides, or inserting of nucleotides within a naturally occurring nucleotide sequence, or at the 5′ or 3′, or both ends of a naturally occurring nucleotide sequence. It is evident that the terms “naturally occurring” and “non-naturally occurring” also apply to peptide or protein sequences. When peptide or protein sequences are envisaged, a non-naturally occurring sequence may be obtained from, or derived from, a naturally occurring sequence by introducing modifications including the changing of amino acids, deleting of amino acids, or inserting of amino acids within a naturally occurring amino acid sequence, or at the N- or C- terminus, or both the N- and C- termini of a naturally occurring amino acid sequence.

“Scrambled sequence” as used herein refers to a scrambled naturally occurring sequence having an identical nucleotide composition as said naturally occurring sequence, but wherein all nucleotides or at least a substantial part of the nucleotides are arranged in an order of occurrence different to the naturally occurring sequence. The term “complementary sequence” is indicative for a nucleotide sequence that is able to form a double-stranded structure with a naturally occurring nucleotide sequence by matching base pairs (i.e. base pairing). A skilled person is aware that base pairing is indicative for a process of binding separate nucleotide sequences by base pairs. A base pair is a unit consisting of two nucleobases bound together by hydrogen bonds. Appropriate geometrical correspondence of hydrogen bond donors and acceptors allows only the “right” pairs to form stably. Examples of base pairs are the A-T (adenine-thymine) base pair, the G-C(guanine-cytosine) base pair, and the U-A (uracil-adenine) base pair.

In certain embodiments wherein the two non-adjacent nucleotide sequences in the second nucleotide arrangement that are naturally occurring Mycoplasma sequences are separated from each other by a nucleotide sequence not-naturally occurring in Mycoplasma, said not-naturally occurring sequence is at least 70%, at least 75%, at least 80%, at least 85%, at least 90%, at least 95%, at least 97% identical, but below 100%, preferably below 99%, more preferably below 98% identical to a nucleotide sequence naturally occurring in a Mycoplasma genomic sequence, preferably the Mycoplasma genomic sequence wherein the oligonucleotide modification system is used in. In certain embodiments, the nucleotide sequence not-naturally occurring in a Mycoplasma genomic sequence encodes an identical amino acid sequence as a naturally occurring Mycoplasma genomic sequence, i.e. in these embodiments the nucleotide sequence not-naturally occurring in a Mycoplasma genomic sequence is said to be codon optimized. In certain embodiments the non-naturally occurring nucleotide sequence encodes at least 2, at least 3, at least 4, at least 5, at least 10, at least 15, at least 20, at least 25 amino acid mutations when compared to the naturally occurring Mycoplasma sequence. In certain embodiments multiple nucleotide-encoded amino acid mutations are adjacent mutations. In further embodiments, no nucleotide-encoded amino acid mutations are adjacent.

In certain embodiments, the second nucleotide arrangement has a nucleotide sequence identity to a naturally occurring Mycoplasma sequence of less than 100%, but preferably at least 60%, at least 65%, at least 70%, at least 75%, at least 80%, at least 85%; at least 90%, at least 95%, at least 97%, at least 99%, based on the total length of the nucleotide sequence of the second nucleotide arrangement.

In further embodiments, the nucleotide sequence not-naturally occurring in Mycoplasma comprises at least one site-specific recombinase target site. Non-limiting examples of recombination target sites suitable for use in an embodiment according to the invention include an FRT site, a KDRT site, a B2RT site, a B2RT site, a RSRT site, a loxP site, a VloxP site, a SloxP site, a vox site, a rox site, an attP site, an attB site, a gix site, a res site, or a site I. In certain embodiments, the site-specific recombination site comprises two 13 nucleotide recognition sequences separated by an 8 nucleotide spacer sequence. Therefore in certain embodiments the site-specific recombination site is a nucleotide sequence with a length of 34 nucleotides. In alternative embodiments the site-specific recombination site comprises a third 13 nucleotide recognition sequence and is separated from a recognition site on the non-spacer end with a single nucleotide. Therefore in certain embodiments the site-specific recombination site is a nucleotide sequence with a length of 48 nucleotides. In certain embodiments, the site-specific recombination target site is a lox site. In further embodiments the site-specific recombination lox site is a loxP site, a VloxP site, a SloxP site, a vox site, or a rox site. In yet further embodiments the site-specific recombination lox site is a loxP site, a VloxP site or a SloxP site. In certain embodiments, the nucleotide sequence not-naturally occurring in Mycoplasma comprises at least one restriction site. In further embodiments, the nucleotide sequence not-naturally occurring in Mycoplasma comprises a nucleotide-encoded selection marker. In further embodiments, the nucleotide sequence not-naturally occurring in Mycoplasma comprises a nucleotide-encoded barcode. In further embodiments, the nucleotide sequence not-naturally occurring in Mycoplasma comprises at least one site-specific recombinase target site and a nucleotide-encoded selection marker. In yet a further embodiment, the nucleotide sequence not-naturally occurring in Mycoplasma comprises a gene drive. A skilled person is aware that a gene drive is a genetic engineering technology that disseminates a particular set of a priori defined genes throughout a population by altering the 50% chance that an allele is transmitted from a parent organism to its offspring. Gene drives have been described extensively (Collins, Gene drives in our future: challenges of and opportunities for using a self-sustaining technology in pest and vector management, BioMedCentral Proceedings, 2018). In further embodiments, the nucleotide sequence not-naturally occurring in Mycoplasma comprises at least one restriction site and/or at least one site-specific recombinase target site and/or at least one nucleotide-encoded selection marker.

“Selection markers” or “selectable markers” as used herein refer to genes or gene products that confer a trait suitable for artificial selection of a cell comprising the marker sequence. Commonly used selection markers are prokaryotic or eukaryotic antiobiotic resistance genes not limited to ampicillin, chloroamphenicol, tetracycline, kanamycine, blasticidine, neomycin, or puromycin. Alternatively, fluorescent markers are envisaged such as (enhanced)GFP or mCherry. In certain embodiments, the nucleotide sequence comprises a dual reporter system combining any of the above mentioned markers, e.g. EGFP/Puromycin resistance gene. In certain embodiments, the nucleotide sequence comprises a toxin or an antitoxin protein. Toxin/antitoxin systems are known to a skilled person (Unterholzner et al., Toxin-antitoxin systems, Mobile genetic elements, 2013). Alternatively, the selection marker is based on a nuclease and nuclease inhibitor system such as the non-limiting example of barnase and barstar (Hartley, Barnase-barstar interaction, Methods in enzymology, 2001).

In certain embodiments, the DNA binding protein comprised in the first nucleotide arrangement is a recombinase, preferably a GP35 recombinase. In further embodiments, the GP35 recombinase is a GP35 recombinase having a nucleotide sequence encoding a protein which is at least 65%, at least 70%, preferably at least 75%, at least 80%, more preferably at least 85%, at least 90%, most preferably 95%, 97%, 99% identical to the amino acid sequence of GP35 from Bacillus subtilis bacteriophage SPP1 as defined by SEQ ID NO: 1 based on the total length of the amino acid sequence of the recombinase. In certain embodiments, the nucleotide sequence of the GP35 recombinase has been optimized for expression in one or more Mycoplasma species. In further embodiments, the nucleotide sequence of the GP35 recombinase has been optimized for expression in M. pneumoniae. In yet alternative embodiments, the sequence of the GP35 recombinase is optimized to maximize the average and/or the median expression level across different Mycoplasma species, i.e. at least two or more distinct Mycoplasma species.

The SPP1 GP35 amino acid sequence annotated under NCBI reference sequence NP_690727.1 is reproduced below (SEQ ID NO: 1):

MATKKQEELKNALAQQNGAVPQTPVKPQDKVKGYLERMMPAIKDVLPKHLDADRLSRIAM NVIRTNPKLLECDTASLMGAVLESAKLGVEPGLLGQAYILPYTNYKKKTVEAQFILGYKGLL DLVRRSGHVSTISAQTVYKNDTFEYEYGLDDKLVHRPAPFGTDRGEPVGYYAVAKMKDGGY NFLVMSKQDVEKHRDAFSKSKNREGVVYGPWADHFDAMAKKTVLRQLINYLPISVEQLSGV AADERTGSELHNQFADDDNIINVDINTGEIIDHQEKLGGETNE

Methods and tools to verify sequence homology or sequence identity between different sequences of amino acids or nucleic acids are well known to a person skilled in the art and include non-limiting tools such as Protein BLAST, ClustalW2, SIM alignment tool, TranslatorX and T-COFFEE. The percentage of identity between two sequences may show minor variability depending on the algorithm choice and parameters. The term “sequence identity” refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence.

“Recombinase” as defined herein is an enzyme capable of effectuating genetic recombination. A skilled person is aware that genetic recombination also known as genetic reshuffling, when used in a context of genetic engineering, refers to an artificial and deliberate recombination of distinct pieces of DNA to create recombinant DNA. Depending on the number and orientation of the DNA sites, there can be an inversion, deletion or insertion of DNA. Recombinases catalyze DNA exchange reactions, which are directionally sensitive, between short target site sequences that are specific to each recombinase. In certain embodiments, the distinct pieces of DNA originate from different organisms. In certain embodiments, one distinct piece of DNA is integrated in the genome of a host cell, and a second distinct piece of DNA is recombinant DNA. In the art, the process describing the use of a recombinase for exchanging DNA sequences is commonly referred to as “recombineering”. Recombineering is an efficient homologous recombination-based method for genome engineering and allows precise insertion, deletion, or any kind of alteration of any DNA sequence (Sharan et al., Recombineering: a homologous recombination-based method of genetic engineering, Nature Protocols, 2009). It is evident to a person skilled in the art that the term “recombinase” as used herein is in its broadest interpretation indicative for any protein that may aid, assist, or contribute to any sort of recombination activity, or provide any sort of recombination activity. Accordingly, in certain embodiments, the recombinase is a catalyzer of DNA exchange reactions. In further embodiments, the recombinase is ssbA or ssbB. In preferred embodiments, the recombinase is a GP35 recombinase as described throughout the present disclosure.

It is well known to a person skilled in the art that different recombinases have been described in eukaryotes, bacteria, archaea, phages and viruses. Both recombinases that only rely on homology between the distinct pieces of DNA and recombinases that only function upon inclusion of (a) specific nucleotide sequence(s) in the distinct pieces of DNA have been described. Several systems comprising site specific recombinases and short target sequences are used in molecular biology. The group of site specific DNA recombinase systems popular for use in genome engineering or synthetic biology includes but is by no means limited to the Hin/hix system, the Cre/lox system, the Flp/FRT system, the XerCD/dif system, the FimBE/fims system, the KD/KDRT system, the B2/B2RT system, the B3/B3RT system, the R/RSRT system, the VCre/VloxP system, the SCre/SloxP system, the Vika/vox system, the Dre/rox system, the λ-Int/attP-attB system, the HK022/attP-attB system, the φC31/attP-attB system, the Bxb1/attP-attB system, the Gin/gix system, the Nigri/nox system, the Panto/pox system and the Tn3 res-siteI system. Different site specific recombinase systems have been reviewed extensively at several occasions (Grindly et al., Mechanisms of site-specific recombination, Annual review of biochemistry, 2006, and Blakely, Mechanisms of horizontal gene transfer and DNA recombination, Molecular Medical Microbiology (Second edition—chapter 15), 2015).

In further embodiments, the nucleotide-encoded GP35 sequence may comprise additional nucleic acids such as those corresponding to or translating into a tag sequence, regulatory sequence, or localization signal. In further embodiments, the sequence comprises additional nucleic acids contributing to the turnover time of the recombinase or to its activity when translated. In yet further embodiments, portions of the sequence naturally occurring have been removed or are changed in position relative to other portions of the sequence. In certain embodiments, the sequence or a portion of the sequence corresponds to the functionally active portion of the GP35 recombinase, wherein said functionally active portion is able to achieve approximately equal, or preferably even higher efficiencies when compared to the corresponding naturally occurring GP35 recombinase, preferably the GP35 recombinase characterized by SEQ ID NO: 1. Also intended herein are nucleotide arrangements in the form of RNA sequences comprising a sequence coding for GP35 recombinases bearing tag sequences, regulatory sequences or localization signals. Further intended are nucleotide arrangements in the form of RNA sequences comprising a sequence coding for mutants of GP35 recombinases bearing tag sequences, regulatory sequences or localization signals, or portions of GP35 recombinases bearing tag sequences, regulatory sequences or localization signals.

In certain embodiments, the set of nucleotide arrangements further comprises a third nucleotide arrangement comprising a promoter or a functional variant or fragment thereof which is active in Mycoplasma bacteria, operably linked to a heterologous nucleotide-encoded gene product.

“Gene product” as used herein is indicative for any molecule directly derived from a nucleotide arrangement. In certain embodiments, the gene product is an RNA molecule. In certain embodiments, the gene product is a polypeptide or protein. A skilled person is aware that the term “gene product” may additionally be indicative for the product derived from a non-naturally occurring operon comprised in a Mycoplasma bacterium, as commonly indicated in the art by the term “heterologous gene product”. The term may therefore cover any protein of biotechnological interest. In certain embodiments, the gene product may be a protein naturally occurring in a distinct Mycoplasma species or in any other organism. The term “heterologous gene product should therefore be interpreted in its broadest interpretation throughout the present disclosure. In certain embodiments where the gene product is a protein, the gene product may contain amino acid mutations when compared to the naturally occurring amino acid sequence of said protein which may impact protein stability or protein activity levels. In certain embodiments, the gene product is a fusion protein comprising multiple proteins or functional fragments thereof. In yet further embodiments, portions of the sequence naturally occurring have been removed or are changed in position relative to other portions of the sequence.

Unless explicitly stated otherwise, reference herein to any peptide, polypeptide, protein, or nucleic acid, or fragment thereof may generally also encompass modified forms of said peptide, polypeptide, protein, or nucleic acid, or fragment thereof, such as bearing post-expression modifications including the following non-limiting examples: phosphorylation, glycosylation, lipidation, methylation, cysteinylation, sulphonation, glutathionylation, acetylation, oxidation of methionine to methionine sulphoxide or methionine sulphone, combinations thereof.

In certain embodiments, the heterologous nucleotide-encoded gene product is polycistronic, preferably bicistronic or tricistronic. The terms “polycistronic”, “bicistronic”, and “tricistronic” as used herein indicate that respectively multiple, two, or three separate proteins are encoded in a single messenger RNA. In certain embodiments, the heterologous nucleotide-encoded gene product comprises a 2A peptide. It has been established in the art that 2A peptides are short peptides that cause produce equimolar levels of multiple genes from the same mRNA. The ribosome skips the synthesis of a peptide bond at the C-terminus of a 2A peptide, leading to separation between the end of the 2A sequence and the next peptide downstream. This skipping occurs between the Glycine and Proline residues found on the C-terminus meaning the upstream cistron will have a few additional residues added to the end, while the downstream cistron will start with the Proline that is part of the 2A sequence (Liu et al., Systematic comparison of 2A peptides for cloning multi-genes in a polycistronic vector, Scientific Reports, 2017). In further embodiments, the one or more 2A peptides comprised in the heterologous nucleotide-encoded gene product are selected from the group of 2A peptides consisting of T2A, P2A, E2A or F2A. In alternative embodiments the heterologous nucleotide-encoded gene product is a fusion protein comprising at least two different polypeptides or proteins linked together. In further embodiment the at least two different polypeptides or proteins are linked directly by a peptide bond connecting the C-terminus of the first polypeptide or protein with the N-terminus of the second polypeptide or protein. In alternative embodiments the at least two different polypeptides or proteins are linked together by a linker sequence. Linker sequences have been described in the art (Chen et al. Fusion protein linkers: property, design and functionality, Advanced Drug Delivery Reviews, 2014). By means of guidance and not limitation, suitable linker sequences include GS, GSS, GGS, GSG, GGGS (SEQ ID NO: 44), GGGGS (SEQ ID NO: 45), GGGGG (SEQ ID NO: 46), EAAAK (SEQ ID NO: 47), EAAAR (SEQ ID NO: 48), AEAAAK (SEQ ID NO: 49), PAPAP(SEQ ID NO: 50), SS, GFLG (SEQ ID NO: 51), LE, GSAT (SEQ ID NO: 52), SEG, or combinations thereof.

The heterologous nucleotide-encoded gene product may be any protein or peptide that has an advantageous effect for the Mycoplasma bacterium, for the infected host, said host possibly being affected by a disease condition such as but not limited to pulmonary infections, or for the environment. In preferred embodiments, the heterologous nucleotide-encoded gene product comprises a therapeutic protein or peptide. The term “therapeutic protein” or “therapeutic peptide” is considered clear to a person skilled in the art and the skilled person understands that a wide range of therapeutic proteins have been described in the art. Therapeutic proteins can be stratified into five large groups: (a) replacing a protein that is deficient or abnormal; (b) augmenting an existing pathway; (c) providing a novel function or activity; (d) interfering with a molecule or organism; and (e) delivering other compounds or proteins, such as a radionuclide, cytotoxic drug, or effector proteins. Alternatively, therapeutic proteins may also be grouped based on their molecular types that include antibody-based drugs, Fc fusion proteins, anticoagulants, blood factors, bone morphogenetic proteins, engineered protein scaffolds, enzymes, growth factors, hormones, interferons, interleukins, and thrombolytics. Therapeutic proteins and therapeutic peptides can also be classified based on their molecular mechanism of activity as (a) binding non-covalently to target such as monoclonal antibodies; (b) affecting covalent bonds such as enzymes; and (c) exerting activity without specific interactions, e.g. serum albumin. The above mentioned classifications and the contribution of each of the groups to the state of the art have been described in scientific literature (Dimitrov, Therapeutic proteins, Methods in Molecular Biology, 2012). Non-limiting examples of classes of therapeutic proteins include cytokines, antibodies, nanobodies, (soluble) receptors, antibody-like protein scaffolds, and functional fragments hereof.

In alternative preferred embodiments, the heterologous nucleotide-encoded gene product comprises an immunogenic protein or a fragment thereof or an immunogenic peptide. A skilled person understands that immunogenic proteins are proteins that are able to elicit or provoke an immune response upon expression in an organism. It is therefore the ability to induce a humoral and/or cell-mediated immune response. Immunogenicity is dependent of different characteristics of an antigen including the non-limiting examples of phylogenetic distance between the immunogenic protein or immunogenic peptide and the host organism, the molecular size of the immunogenic protein since larger proteins are generally observed to be more immunogenic, the epitope density of the immunogenic protein, protein structure, and degradability. Methods have been described in the art to estimate the immunogenicity score of a protein (Baker et al., Immunogenicity of protein therapeutics, Self/nonself Immune Recognition and Signaling, 2010). Also envisaged are known immunogenic proteins described in the art or a fragment thereof. In certain embodiments, the heterologous nucleotide-encoded gene product is an interleukin or antigen.

In further embodiments where the gene product is a protein said gene product may further comprise a nucleotide-encoded peptide or protein tag sequence. Non-limiting examples of commonly used peptide tag sequences are the AviTag, C-tag, calmodulin-tag, polyglutamate tag, E-tag, Flag-tag, HA-tag, His-tag, Myc-tag, NE-tag, Rho1D4-tag, S-tag, SBP-tag, Softag 1, Softag 3, Spot-tag, Strep-tag, TC tag, Ty tag, V5 tag, VSV-tag, Xpress tag, isopeptag, SpyTag, SnoopTag, DogTag, and the SdyTag. In further embodiments, the gene product comprises at least two nucleotide-encoded peptide or protein tag sequences.

In certain embodiments, the heterologous nucleotide-encoded gene product further comprises an exposure signal sequence. Exposure signal sequences have been defined in the state of the art and are known to a skilled person. In further embodiments, the exposure signal sequence is a naturally occurring sequence in Mycoplasma, preferably M pneumoniae. In yet further embodiments, the exposure signal sequence is a Mycoplasma, preferably M pneumoniae exposure signal sequence. In alternative embodiments, the exposure signal sequence is a not-naturally occurring Mycoplasma sequence.

In certain embodiments, the heterologous nucleotide-encoded gene product comprises a secretion signal sequence. In further embodiments, the secretion signal sequence is a naturally occurring sequence in Mycoplasma, preferably M. pneumoniae. In yet further embodiments, the secretion signal sequence is a Mycoplasma, preferably M pneumoniae secretion signal sequence. In alternative embodiments, the secretion signal sequence is a not-naturally occurring Mycoplasma sequence. Mycoplasma secretion signals have been described in International patent application WO2016/135281 and are therefore known to a person skilled in the art. A skilled person furthermore understands that mutagenized exposure or secretion signals may be further mutagenized to improve exposure or secretion respectively of the heterologous nucleotide-encoded gene product. In certain embodiments, concatenated secretion signals are comprised in the nucleotide-encoded gene product. In certain embodiments, a plurality of distinct secretion signals is comprised in the nucleotide-encoded gene product. In further embodiments, different secretion signals are comprised at different locations of the nucleotide-encoded gene product. In embodiments wherein the heterologous nucleotide-encoded gene product is polycistronic, the polycistronic sequence may contain both at least one secretion signal sequence and at least one exposure signal sequence.

The terms “exposure signal sequence” and “secretion signal sequence” as used herein are indicative for sequences encoding exposure or secretion signal peptides that targets the linked protein for exposure on the cell membrane of extracellular secretion respectively. Afterwards, the signal sequence may be removed from the linked protein by proteolytic cleavage. In certain embodiments, the exposure or secretion signal sequence is located at the N-terminus of the nucleotide-encoded gene product, here a protein.

In alternative embodiments, the third nucleotide arrangement comprised in the set of nucleotide arrangements comprises a promoter or functional variant or fragment thereof which is active in Mycoplasma bacteria, and which promoter is operably linked to a nucleotide-encoded nuclease or a nucleotide-encoded recombinase. In certain embodiments, the nuclease or recombinase is a protein. In certain embodiments the recombinase comprised in the third nucleotide arrangement is distinct from the recombinase comprised in the first nucleotide arrangement. In further embodiments wherein the third nucleotide arrangement comprises a recombinase, said recombinase preferably is a Cre recombinase, a VCre recombinase, or a SCre recombinase. In further embodiments wherein the third nucleotide arrangement comprises a nuclease, said nuclease may be an artificial nuclease originating from the fusion of a DNA recognition domain and a catalytic domain. In alternative embodiments, the nuclease is a catalytic RNA molecule, preferably a ribozyme. In certain embodiments, the nucleotide-encoded nuclease is not functional in the absence of another molecule capable of interacting with the nuclease in the host cell. In certain embodiments, the nucleotide-encoded nuclease has been codon optimized for expression in Mycoplasma, preferably M. pneumoniae.

Tools and protocols that allow codon optimization of a nucleotide sequence for expression in a host organism have been described in the art at numerous occasions in the art (Ayyar et al., Optimizing antibody expression: the nuts and bolts. Methods. 2017, and Kaur J. et al., Strategies for optimization of heterologous protein expression in E. coli: Roadblocks and reinforcements, International Journal of Biological Macromolecules, 2018).

The term “nucleases” as used herein, also known as “nucleodepolymerases” and “polynucleotidases” are a group of enzymes that effectuate cleavage of phosphodiester bonds between the nucleotides of nucleic acids (i.e. molecular scissors). Both nucleases that are capable of inducing single stranded breaks and double stranded breaks have been described in the art. It is known that there is a large diversity in terms of structure and function among different nucleases. Nucleases can be either endonucleases that lead to the generation of oligonucleotides as a consequence of their activity, or exonucleases that have single nucleotides as cleavage products (i.e. exerting their enzymatic activity from the 5′ or 3′ end of a oligonucleotide).

“Ribozymes” as defined herein are RNA molecules that are capable of catalyzing specific biochemical reactions. Non limiting examples of ribozymes include RNaseP, Peptidyl transferase 23S rRNA, GIR1 branching ribozyme, leadzyme, Group I introns, Group II introns, Hairpin ribozyme, Hammerhead ribozyme, HDV ribozyme, VS ribozyme, Mammalian CPEB3 ribozyme, CoTC ribozyme and glmS ribozyme.

In further embodiments where the third nucleotide arrangement comprises a nucleotide-encoded nuclease, the nuclease is an endonuclease. In yet further embodiments, the endonuclease is selected from the group comprising of restriction enzymes, meganucleases, zinc-finger nucleases (ZFNs), transcription-activator like effector nucleases (TALENs), and CRISPR-associated (Cas)-based nucleases.

The term “restriction enzyme” as used herein and which can be used interchangeably with “restriction endonuclease” or “restrictase” refers to a subgroup of endonucleases that cleave DNA at or in close proximity of specific recognition sites, which have been termed restriction sites in the art. A fragment resulting from the cutting of a DNA strand by a restriction enzymes is known as a restriction fragment.

A large body of different restriction enzymes has been identified in the art. A skilled person is aware of tools and databases that are available to find information on both restriction enzyme activity and restriction sites (Roberts et al., REBASE—enzymes and genes for DNA restriction and modification, Nucleic Acids Research, 2007).

“Meganucleases” are a group of nucleases that are characterized by a large recognition site, typically between 12 and 40 base pairs. Due to its size, the recognition site of meganucleases is unique or near unique for any given genome. Meganucleases have been identified in a large number of organisms including Archaea, bacteria, phages, fungi, yeast, algae, plants with unique recognition sites. Furthermore, tools to produce artificial meganucleases have been described in the art (Bartsevich, et al., Meganucleases as an efficient tool for genome engineering, Molecular Therapy, 2016). In certain embodiments, the meganuclease is selected from the group consisting essentially of one of the following families (based on sequence and structure motif): LAGLIDADG, GIY-YIG, HNH, His-Cys box, PD-(D/E)XK. In certain embodiments the meganuclease is an intron-encoded nuclease. In further embodiments the meganuclease is selected from the LAGLIDADG meganuclease family, wherein preferably the meganuclease is Sce-J.

“Zinc-finger nucleases”, abbreviated as ZFNs, are artificial restriction enzymes that comprise a zinc finger DNA binding domain fused to a DNA cleavage domain. Zinc finger domains can be engineered to target specific desired DNA sequences and this enables zinc finger nucleases to target unique sequences within any given genome. A typical zinc finger DNA binding domain includes between three and six zinc finger repeats that are each capable of recognizing 9 to 18 basepairs. Diverse methods to generate zinc finger arrays capable of targeting desired sequences have been explored, have been publicly described and are therefore known to the skilled person (Wu et al., Custom-designed zinc finger nucleases: What is Next?., Cellular and molecular life sciences, 2007). A non-limiting example of a suitable non-specific cleavage domain is the obligate dimeric endonuclease FokI and FokI domains with enhanced cleavage activity such as Sharkey (Guo et al., Directed evolution of an enhanced and highly efficient FokI cleavage domain for zinc finger nucleases, Journal of molecular biology, 2010). Obligate heterodimeric ZFNs have been designed that contain FokI domains comprising modified dimerization interfaces whereby only the intended heterodimeric species possess a catalytic activity (Szczepek et al., Structure-based redesign of the dimerization interface reduces the toxicity of zinc-finger nucleases, Nature Biotechnology, 2007).

“Transcription-activator like effector nucleases”, abbreviated as TALENs, are artificial restriction enzymes that comprise a Transcription Activator Like (TAL) effector DNA binding domain fused to a DNA cleavage domain. Similar to zinc fingers, TAL effector domains can be engineered to bind any given DNA sequence present in any genome. Tal effector domains contain a repeated highly conserved 33 or 34 amino acid sequence with variable amino acids at the 12^{th} and 13^{th} position, commonly annotated as repeat variable diresidues. These repeat variable diresidues are highly variable and show a strong correlation with specific nucleotide recognition. Tools and protocols to generate TAL effector domains specific for a desired sequence are publicly available (Heigwer et al., E-TALEN: a web tool to design TALENs for genome engineering, Nucleic acids research, 2013, and Neff et al., Mojo Hand, a TALEN design tool for genome editing applications, BioMedCentral Bioinformatics, 2013). In accordance to the above-described ZFNs, TALENs typically use a (modified) FokI domain as DNA cleavage domain.

The term “CRISPR-associated (Cas)-based nucleases”, which may be used interchangeably with “CRISPR/Cas nuclease” refers to an enzyme that relies on the use of Clustered Regularly Interspaced Short Palindromic Repeats (CRISPR) sequences to recognize and cleave specific strands of nucleotide sequences that are complementary to the CRISPR sequence. CRISPR/Cas is a prokaryotic immune system conferring a prokaryotic defense mechanism to foreign nucleotide sequences. CRISPR/Cas systems are commonly regarded in the state of the art as a prokaryotic acquired immune system. Both single stranded, double stranded, RNA and DNA cleaving CRISPR/Cas systems have been described in the art and are therefore known to a person skilled in the art (Makarova and Koonin, Annotation and classification of CRISPR-Cas systems, Methods in molecular biology, 2015, and Makarova et al., Classification and nomenclature of CRISPR-Cas systems: where from here?, The CRISPR journal, 2018). Non-limiting examples of Cas proteins include Cas3, Cas 8a, Cas5, Cas8b, Cas8c, Cas1Od, Csel, Cse2, Csy1, Csy2, Csy3, GSU0054, CasIO, Csm2, Cmr5, CasIO, Csx11, Csx10, Csfl, Cas9, Csn2, Cas4, Cpf1 (i.e. Cas12), C2c1, C2c3, Cas13a, Cas13b, Cas13c, and Cas13d. A skilled person is able to appreciate that different Cas proteins may require different CRISPR sequences. It is furthermore known that CRISPR/Cas activity can be inhibited by the use of anti-CRISPR molecules, preferably anti-CRISPR proteins.

In certain embodiments, the Cas-based nuclease is a type II nuclease. In further embodiments, the Cas-based nuclease is a Cas9 nuclease. In even further embodiments, the Cas-based nuclease is a Cas9 nuclease characterized by a nucleotide sequence encoding a protein which is at least 65% identical, at least 70% identical, preferably at least 75% identical, at least 80% identical, more preferably at least 85% identical, at least 90% identical, most preferably at least 95% identical, at least 97% identical, at least 99% identical to the amino acid sequence of Cas9 from Streptococcus pyogenes as defined by SEQ ID NO: 2 based on the total length of the amino acid sequence of the Cas9 or functional variant thereof. In certain embodiments, the nucleotide sequence encoding the Cas9 protein is codon optimized for expression in Mycoplasma, preferably M pneumoniae. In alternative embodiments, the Cas9 protein is a modified Cas9 protein comprising a mutagenized HNH and/or RuvC catalytic domain. In further embodiments, the Cas9 protein is a nickase Cas9 (nCas9). In yet alternative embodiments, the Cas9 protein is a catalytically inactive Cas9 (i.e. dead Cas9, or dCas9). In such embodiments, double stranded breaks in Mycoplasma may be achieved by fusing the catalytically inactive Cas9 protein to a DNA cleavage domain such as the non-limiting example FokI.

Cas9 is a RNA-guided DNA endonuclease enzyme originally identified in Streptococcus pyogenes. Cas9 is characterized by a bi-lobed architecture, an alpha helical lobe (i.e. a recognition lobe) and a nuclease lobe, the two lobes being connected by a bridge helix. Cas9 comprises two nuclease domains, a RuvC domain and a HNH nuclease domain, which are responsible for cleavage of the non-target DNA strand and the target strand respectively. The CRISPR/Cas9 system has the capacity to recognize any DNA sequence that comprises a protospacer adjacent motif (PAM), with the PAM being the nucleotide sequence NGG, wherein N may be any nucleotide. A skilled person understands that a functional Cas9 relies on complex formation of the protein with CRISPR RNA (crRNA) and trans-activating crRNA (tracrRNA).

Alternatively to the crRNA and tracrRNA, Cas9 may function by interaction with a single guide RNA (gRNA) sequence. Single guide RNA sequences have been designed artificially and are able to replace the crRNA and tracrRNA. It is known that the single guide RNA is a single RNA chimera of tracrRNA and crRNA. The crRNA or gRNA sequence comprises the sequence which the Cas9 will be targeted to by conventional base pairing of the crRNA or gRNA with the target sequence. Hence in certain embodiments described herein, the crRNA sequence or target specific portion of the gRNA sequence has a length of 15, 16, 17, 18, 19, 20, 21, 22, 23, 24, 25, 26, 27, 28, 29, or 30 nucleotides.

Tools for designing gRNA sequences have been reported in the art. Non-limiting examples of CRISPR-Cas design tools, optionally comprising an off-target analysis module include Breaking-Cas, Cas-OFFinder, CASTING, CCTop, CHOPCHOP, CHOPCHOP v2, CRISPOR, CRISPR Design, CRISPRdirect, CRISPRscan, CRISPRseek, DESKGEN, GuideScan, GT-Scan, Off-Spotter, sgRNA Designer, Synthego Design Tool, TUSCAN, and VARSCOT.

The S. pyogenes Cas9 amino acid sequence annotated under NCBI Reference sequence WP_001040087.1 is reproduced below (SEQ ID NO: 2):

MNKPYSIGLDIGTNSVGWSIITDDYKVPAKKMRVLGNTDKEYIKKNLIGA
LLFDGGNTAADRRLKRTARRRYTRRRNRILYLQEIFAEEMSKVDDSFFHR
LEDSFLVEEDKRGSKYPIFATLQEEKDYHEKFSTIYHLRKELADKKEKAD
LRLIYIALAHIIKFRGHFLIEDDSFDVRNTDISKQYQDFLEIFNTTFENN
DLLSQNVDVEAILTDKISKSAKKDRILAQYPNQKSTGIFAEFLKLIVGNQ
ADFKKYFNLEDKTPLQFAKDSYDEDLENLLGQIGDEFADLFSAAKKLYDS
VLLSGILTVIDLSTKAPLSASMIQRYDEHREDLKQLKQFVKASLPEKYQE
IFADSSKDGYAGYIEGKTNQEAFYKYLSKLLTKQEDSENFLEKIKNEDFL
RKQRTFDNGSIPHQVHLTELKAIIRRQSEYYPFLKENQDRIEKILTFRIP
YYIGPLAREKSDFAWMTRKTDDSIRPWNFEDLVDKEKSAEAFIHRMTNND
FYLPEEKVLPKHSLIYEKFTVYNELTKVRYKNEQGETYFFDSNIKQEIFD
GVFKEHRKVSKKKLLDFLAKEYEEFRIVDVIGLDKENKAFNASLGTYHDL
EKILDKDFLDNPDNESILEDIVQTLTLFEDREMIKKRLENYKDLFTESQL
KKLYRRHYTGWGRLSAKLINGIRDKESQKTILDYLIDDGRSNRNFMQLIN
DDGLSFKSIISKAQAGSHSDNLKEVVGELAGSPAIKKGILQSLKIVDELV
KVMGYEPEQIVVEMARENQTTNQGRRNSRQRYKLLDDGVKNLASDLNGNI
LKEYPTDNQALQNERLFLYYLQNGRDMYTGEALDIDNLSQYDIDHIIPQA
FIKDDSIDNRVLVSSAKNRGKSDDVPSLEIVKDCKVFWKKLLDAKLMSQR
KYDNLTKAERGGLTSDDKARFIQRQLVETRQITKHVARILDERFNNELDS
KGRRIRKVKIVTLKSNLVSNFRKEFGFYKIREVNNYHHAHDAYLNAVVAK
AILTKYPQLEPEFVYGDYPKYNSYKTRKSATEKLFFYSNIMNFFKTKVTL
ADGTVVVKDDIEVNNDTGEIVWDKKKHFATVRKVLSYPQNNIVKKTEIQT
GGFSKESILAHGNSDKLIPRKTKDIYLDPKKYGGFDSPIVAYSVLVVADI
KKGKAQKLKTVTELLGITIMERSRFEKNPSAFLESKGYLNIRADKLIILP
KYSLFELENGRRRLLASAGELQKGNELALPTQFMKFLYLASRYNESKGKP
EEIEKKQEFVNQHVSYFDDILQLINDFSKRVILADANLEKINKLYQDNKE
NISVDELANNIINLFTFTSLGAPAAFKFFDKIVDRKRYTSTKEVLNSTLI
HQSITGLYETRIDLGKLGED

In certain embodiments, the third nucleotide arrangement comprises a nucleotide-encoded hyperactive Cas9 protein. Hyperactive Cas9 proteins have been described in the art and are known to a person skilled in the art. Non-limiting examples of hyperactive Cas9 proteins are HypaCas9, eCas9, and HiFiCas9.

In certain embodiments, at least two nucleases are comprised in the third nucleotide arrangement. In further embodiments, at least two different nucleases are comprised in the third nucleotide arrangement. In certain embodiments, the one or more nucleases have been codon optimized for expression in Mycoplasma, preferably M pneumoniae. In certain embodiments, the one or more nuclease targets a naturally occurring sequence of Mycoplasma. In alternative embodiments, the one or more nuclease targets a sequence comprise in any other nucleotide arrangement described herein. In yet alternative embodiments, the one or more nuclease targets (a part of) the nucleotide sequence comprised in the third nucleotide arrangement that encodes the one or more nuclease.

In certain embodiments, the set of nucleotide arrangements comprises a fourth nucleotide arrangement comprising a single guide RNA sequence capable of base pairing with a naturally occurring sequence in a Mycoplasma genome. In alternative embodiments, the set of nucleotide arrangements comprises a fourth nucleotide arrangement comprising at least a tracrRNA sequence and optionally a crRNA sequence capable of base pairing with a naturally occurring sequence in a Mycoplasma genome. In further embodiments, the crRNA sequence may be a pre-crRNA sequence. In alternative embodiments, the set of nucleotide arrangements comprises a fourth nucleotide arrangement comprising at least a crRNA sequence and optionally a tracrRNA sequence capable of base pairing with a naturally occurring sequence in a Mycoplasma genome. In further embodiments, the fourth nucleotide arrangement comprises at least two different single guide RNA sequences or at least two different crRNA sequences capable of base pairing with a naturally occurring sequence in a Mycoplasma genome. In certain embodiments, multiple iterations of a fourth nucleotide arrangement are included in the set of nucleotide arrangements each encoding a different single guide RNA or crRNA sequence capable of base pairing with a naturally occurring sequence in a Mycoplasma genome. In certain embodiments, multiple iterations of a fourth nucleotide arrangement are included in the set of nucleotide arrangements each encoding a single guide RNA or crRNA and/or tracrRNA sequence tailored for interaction with a different Cas protein. In certain embodiments, the fourth nucleotide arrangement further comprises a promoter sequence, preferably a U6 promoter or a T7 promoter. In certain embodiments the fourth nucleotide arrangement is an RNA molecule. In further embodiments the fourth nucleotide arrangement is a crRNA-tracrRNA duplex.

In certain embodiments, at least one nucleotide arrangement comprises a promoter with a nucleotide sequence of at least 65% identity, at least 70% identity, preferably at least 75% identity, at least 80% identity, more preferably at least 85% identity, at least 90% identity, most preferably at least 95% identity, at least 97% identity to the nucleotide sequence of SEQ ID NO: 3. In alternative embodiments, the at least two, at least three, at least four nucleotide arrangements comprise a promoter with a nucleotide sequence of at least 65% identity, at least 70% identity, preferably at least 75% identity, at least 80% identity, more preferably at least 85% identity, at least 90% identity, most preferably at least 95% identity, at least 97% identity to the nucleotide sequence of SEQ ID NO: 3.

The SEQ ID NO: 3 promoter nucleotide sequence is reproduced below:

5′-TAGTATTTAGAATTAATAAAGT-3′

In certain embodiments, at least one nucleotide arrangement further comprises a regulatory sequence capable of modulating transcription. In certain embodiments, the regulatory sequence capable of modulating transcription is an enhancer sequence. In further embodiments, the regulatory sequence capable of modulating transcription is a riboswitch.

“Riboswitch” as defined herein is a regulatory sequence comprised in messenger RNA that may bind to a small molecule, wherein said binding has as consequence a change in the production of the one or more proteins encoded by the messenger RNA. A riboswitch is commonly divided into two parts: an aptamer and an expression platform. The aptamer directly binds a small molecule, and the expression platform undergoes structural changes in response to the changes in the aptamer. The expression platform is what regulates gene expression. Depending on the type of riboswitch, binding by a small molecule may enable translation, or inhibit translation. Non-limiting examples of riboswitches include cobalamin riboswitches, cyclic AMP-GMP riboswitches, cyclic di-AMP riboswitches, cyclic di-GMP riboswitches, fluoride riboswitches, FMN riboswitches, glnS riboswitches, glutamine riboswitches, glycine riboswitches, lysine riboswitches, manganese riboswitches, NiCo riboswitches, preQ1 riboswitches, purine riboswitches, SAH riboswitches, SAM riboswitches, SAM-SAH riboswitches, tetrahydrofolate riboswitches, TPP riboswitches, ZMP/ZTP riboswitches and the Moco RNA motif, the latter which is presumed to be a riboswitch. In certain embodiments, each promoter-containing nucleotide arrangement comprises a different riboswitch. In alternative embodiments, each promoter-containing nucleotide arrangement comprises a different riboswitch. In yet alternative embodiments, at least one nucleotide arrangement comprises two different riboswitches.

“Small molecule” as used herein is indicative for compounds, preferably organic compounds, with a size comparable to those organic molecules generally used in pharmaceuticals. Small organic molecules range in size up to about 5000 Da, e.g., up to about 4000 Da, preferably up to 3000 Da, more preferably up to 2000 Da, even more preferably up to about 1000 Da, e.g., up to about 900, 800, 700, 600 or up to about 500 Da.

Accordingly an aspect of the invention is directed to the use of a GP35 recombinase for altering the genomic sequence of a Mycoplasma bacterium. In certain embodiments, the GP 35 recombinase is a GP35 recombinase having a nucleotide sequence encoding a protein which is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the recombinase. In certain embodiments, the GP35 recombinase is introduced into the Mycoplasma bacterium as a nucleotide arrangement comprising a sequence encoding for said GP35 recombinase. In further embodiments the nucleotide arrangement is a DNA sequence. In alternative embodiments the nucleotide arrangement is a RNA sequence. In alternative embodiments the nucleotide arrangement is a recombinant protein. In certain embodiments the GP35 recombinase is used in combination with a chemical agent or recombinant protein that increases homologous recombination in bacteria, preferably in Mycoplasma bacteria. In yet alternative embodiments the GP35 recombinase is integrated in the genomic sequence of a Mycoplasma bacterium. In certain embodiments, the GP35 recombinase is used for deletion of a part of the genomic sequence of a Mycoplasma bacterium, i.e. for generation of a knock-out Mycoplasma strain. In certain embodiments, the GP35 recombinase is used for insertion of a non-naturally occurring sequence in the genomic sequence of a Mycoplasma bacterium. In further embodiments the GP35 recombinase is used for insertion of a non-naturally occurring sequence encoding a gene product in a Mycoplasma bacterium. In yet further embodiments the GP35 recombinase is used for insertion of a non-naturally occurring sequence encoding a gene product in a Mycoplasma bacterium. In alternative embodiments the GP35 recombinase is used for simultaneous deletion of a part of the genomic sequence and insertion of a non-naturally occurring sequence in the genomic sequence of a Mycoplasma bacterium.

In any of the embodiments described herein concerning the modification system, methods, kits, and uses, it is evident that the DNA binding protein, preferably a DNA recombinase, more preferably a GP35 recombinase, even more preferably a GP35 recombinase having an amino acid sequence which is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the GP35 recombinase, may be comprised in and/or used in its protein form, i.e. the translated product.

Using the protein form of the DNA binding protein, preferably the GP35 DNA recombinase, may entail several advantages over using an oligonucleotide sequence encoding said protein:
1) The gene encoding DNA binding protein is not comprised in the genome. By using the protein form, the protein is not constitutively expressed and will not generate a metabolic load. Furthermore, insertion of the DNA binding protein in the genome might undesired side effects associated to unexpected or unpredicted genetic variations of the strain. An inserted gene can be removed but it would imply additional steps in the engineering, requiring also another resistance marker insertion and posterior elimination by using e.g. the Cre-Lox system which is time consuming.
2) Reduction in the number of transformations to be performed. Since the gene is not inserted and the protein is degraded, cells do not maintain the Gp35 protein. No puromycine marker is needed for selection.
3) It can be used in any strain independently of the genetic background or resistance markers included in the genome. Since no selection by inclusion of GP35 is required the protein can be included in any proteins with any genetic background. Thus, more rounds of depletions can be done, reducing also the timing in the these protocols.
4) The protocol could be extrapolated to other proteins like Cre, vCre, Cas9, transposase, Scel. The same procedure could be used to transform with other proteins.
5) Using the protein form bypasses any problems with respect to translation difficulties that are commonly observed when introducing foreign genes in an organism. There is no dependency on the translation machinery of the transformed cells.

In general, the present genome modification approach could be a safety mechanism to maintain the cell for some rounds of division. The system could be used to deplete essential genes or to enhance their replacement. The protein encoded by essential gene (the essential protein) could be transformed in a strain wherein GP35 is expressed, either by insertion of said recombinase GP35 in the genome or introduced into the Mycoplasma cell by means of an RNA sequence or (recombinant) GP35 protein, together with the oligo that directs the depletion or replacement of the essential gene of interest.

Subsequently, the essential gene would be depleted. Hence, the essential gene and any of its gene products would be eliminated from the cell, hereby generating a lethal strain, which requires one or more external supplements in order to be able to survive and/or propagate. While several genome engineering methods may be used to generate lethal versions of a given genome, the paucity of Mycoplasma engineering methods hampered the generation of lethal Mycoplasma strains. The present invention enables to generate lethal Mycoplasma strains.

Therefore, in certain embodiments of the disclosure when GP35 recombinase protein is used, the protein may be GP35 recombinase protein that is recombinantly produced in any suitable production cell line. Recombinant protein production and purification strategies have been described in great detail at numerous occasions throughout the art (e.g. Structural Genomics Consortium, Protein production and purification, Nature Methods, 2011). In certain embodiments, the recombinant GP35 protein may be introduced as two or more complementary protein portion (i.e. fragments) that only lead to recombinase activity in a host cell when complemented with each other to form a functional GP35 recombinase. In further embodiments, each GP35 recombinase fragment may be physically linked to one part of an inducible complementation system wherein complementation is obtained by addition of a stimulus (e.g. light) or a chemical compound. In such embodiments, the recombineering event may be considered as a data recording means in the genomic sequence of a Mycoplasma bacterium. In certain methods that are presented throughout the present disclosure wherein recombinant GP35 recombinase is used, the GP35 recombinase may be introduced simultaneously with the oligonucleotide arrangement that is to be recombineered into the genome of the Mycoplasma bacterium, or at another point in time (i.e. before or after addition of said nucleotide arrangement). In certain embodiments, a preformed GP35-oligunucleotide arrangement may be formed in vitro before introducing the GP35 recombinase (complex) to the target Mycoplasma bacterium. In alternative embodiments, the GP35 recombinase and the oligonucleotide arrangement to be recombineered into the Mycoplasma genome may be formed after introduction of the GP35 recombinase into said bacterium. Introduction of GP35 recombinase into the target bacterium may be performed by any suitable methods as a skilled person readily appreciates. A non-limiting example of such a method is electroporation.

A further aspect of the invention is directed to a method of altering the genome of a Mycoplasma bacterium wherein the method comprises introducing an oligonucleotide modification system as described herein or at least one of the nucleotide arrangements described herein into the Mycoplasma bacterium, preferentially into M pneumoniae. Additionally, an aspect of the invention is directed to a method of altering the genome of a Mycoplasma bacterium wherein the method comprises introducing at least one set of the nucleotide arrangements described herein into the Mycoplasma bacterium.

Methods and protocols to introduce nucleotide arrangements into bacteria, i.e. methods of bacterial transformation, are known to a person skilled in the art (Johnston et al., Bacterial transformation: distribution, shared mechanisms and divergent control, Nature reviews Microbiology, 2014). The term “transformation” is indicative for a genetic alteration of a cell resulting from the direct uptake and incorporation of exogenous genetic material. Transformation is a horizontal gene transfer process and is commonly used in context of introducing foreign DNA to a bacterial, yeast, plant, animal, or human cell. Cells capable of taking up foreign DNA are named competent cells. In other embodiments, transformation may be indicative for the insertion of new genetic material into animal and human cells, albeit the term “transfection” is more common for these cells.

Non-limiting examples of suitable transformation methods that can be applied to bacteria include heat-shock transformation and electroporation. In heat shock transformation, artificial competence is typically induced by making the cell permeable to DNA by subjecting them to non-physiological conditions. In such a typical transformation experiment, the cells are incubated in a solution containing divalent cations often in cold conditions, before the cells are exposed to a heat shock. It is theorized that exposure of the cells to divalent cations are responsible for a weakening of the cell surface structure, rendering it (more) permeable to DNA. The heat shock generates a thermal imbalance across the membrane, forcing entry of DNA through cell pores (i.e. adhesion zones or Bayer junctions) or through the damaged cell wall. An alternative method to induce transformation is by means of electroporation, which is hypothesized to create pores in the cellular membrane. In electroporation the bacterial cells are briefly exposed to an electric field of 10-20 kV/cm. After the shock, cellular membrane repair mechanisms remove the pores.

Methods to transform Mycoplasma bacteria, albeit historically a more difficult to transform genus of bacteria, have also been described in the art (Minion and Kapke, Transformation of Mycoplasmas, Mycoplasma protocols, 1998). Transformation protocols specific forM. pneumoniae are also part of the state of the art (Krishnakumar et al., Targeted chromosomal knockouts in Mycoplasma pneumoniae, Applied and environmental microbiology, 2010).

In certain embodiments, the method comprises introduction of at least one protein that is part of the oligonucleotide modification system as described herein together with at least one nucleotide arrangement that is part of the oligonucleotide modification system as described herein. In certain embodiments, the method comprises sequential introduction of at least one nucleotide arrangement. In certain embodiments, the method comprises sequential introduction of any one of the first, second, third and/or fourth nucleotide arrangements as described herein in any order of sequence. In certain embodiments, the method further comprises the introduction of a chemical agent, recombinant protein or nucleotide sequence beneficial for genomically modifying bacteria, preferably Mycoplasma bacteria in addition to the introduction of a set of nucleotide arrangements to the Mycoplasma bacterium. In certain embodiments the chemical agent, recombinant protein or nucleotide sequence influences the Mycoplasma growth rate, preferably induces a faster Mycoplasma growth rate. In certain embodiments, the method comprises culturing the Mycoplasma bacterium in a culture medium optimized for generation of genomically modified Mycoplasma bacteria. In certain embodiments, the method comprises culturing the Mycoplasma bacterium in serum-free medium.

In certain embodiments, the method comprises enrichment and/or isolation of Mycoplasma bacteria from a subject. In further embodiments, the subject is a human subject. In certain embodiments, the method comprises targeted sequencing of at least one genomic region in the Mycoplasma bacterium prior to the introduction of at least one nucleotide arrangement as described herein or prior to the introduction of a set of nucleotide arrangements as described herein. In certain embodiments, the method comprises targeted sequencing of at least one genomic region in the Mycoplasma bacterium after the introduction of at least one nucleotide arrangement as described herein or after the introduction of a set of nucleotide arrangements as described herein. In certain embodiments, the method comprises sequencing of the complete genome of the Mycoplasma bacterium after introduction of at least one nucleotide arrangement as described herein or after the introduction of a set of nucleotide arrangements as described herein. In certain embodiments the method comprises lyophilization of the Mycoplasma bacteria after introducing of at least one nucleotide arrangement as described herein or after the introduction of a set of nucleotide arrangements as described herein.

The term “lyophilization” which may be used interchangeably with terms such as “freeze-drying” and “cryodesiccation” may be used interchangeably herein and refers to dehydration process which involves freezing the product (i.e. Mycoplasma bacteria) without destroying the physical structure of the matter. Lyophilisation comprises at least a freezing step and a sublimation step. The sublimation step may comprise two stages of drying: a primary drying step and a secondary drying step. Advantages of lyophilisation may be but are not limited to improved aseptic handling, enhanced stability of a dry powder, the removal of water without excessive heating of the product, and enhanced product stability in a dry state. In general, the quality of a rehydrated, lyophilized product is excellent and does not show an inferior quality to a non-lyophilized product. In context of the invention, quality of the Mycoplasma bacterium may refer to any of the following non-limiting examples: growth rate, morphology, virulence, expression levels of heterologous nucleotide-encoded gene products, and metabolite production.

In certain embodiments, the method further comprises subjecting the Mycoplasma bacterium to a site-specific recombinase reaction. In certain embodiments, the site-specific recombinase is introduced as a protein. In the methods for transformingMycoplasma, a protein may be co-transformed with a nucleic acid. In certain embodiments, the site specific recombinase required for the site-specific recombination reaction is introduced to the Myoplasma bacterium after the introduction of at least one nucleotide arrangement as described herein or after the introduction of a set of nucleotide arrangements as described herein. In further embodiments, a site specific recombinase required for the site-specific recombinase reaction is introduced to the Myoplasma bacterium after the introduction of at least one second nucleotide arrangement as described herein or after the introduction of a set of nucleotide arrangements comprising a second nucleotide as described herein. In yet even further embodiments the recombinant protein comprises a cell penetrating peptide. In yet even further embodiments the cell penetrating peptide is a peptide that selectively penetrates bacterial cells. In alternative embodiments the site-specific recombinase is introduced as a RNA molecule. In alternative embodiments the site-specific recombinase is introduced as a DNA molecule. In certain embodiments the method comprises culturing the Mycoplasma bacteria in a fed-batch incubator. In certain embodiments the method comprises culturing the Mycoplasma bacteria in a co-culture. In further embodiments the co-culture comprises Mycoplasma bacteria and mammalian cells. In yet further embodiments the co-culture comprises Mycoplasma bacteria and human cells.

In more specific embodiments, the method comprises the introduction of a first and second nucleotide arrangement as defined herein in the Mycoplasma bacterium, wherein the first nucleotide arrangement comprises a nucleotide-encoded recombinase, and the second nucleotide arrangement comprises two non-adjacent naturally occurring Mycoplasma sequences each having a minimum length of 5 nucleotides, optionally comprising a site-specific recombinase target site and/or a nucleotide-encoded selection marker. In another embodiment, the method comprises the introduction of a first, second and third nucleotide arrangement as defined herein in the Mycoplasma bacterium, wherein the first nucleotide arrangement comprises a nucleotide-encoded recombinase, the second nucleotide arrangement comprises two non-adjacent naturally occurring Mycoplasma sequences each having a minimum length of 5 nucleotides, optionally comprising a site-specific recombinase target site and/or a nucleotide-encoded selection marker, and the third nucleotide arrangement comprises a heterologous nucleotide-encoded gene product, optionally wherein the heterologous nucleotide-encoded gene product comprises a therapeutic protein (or peptide) or an immunogenic protein (or peptide). In a further alternative embodiment, the method comprises the introduction of a first, second and third nucleotide arrangement as defined herein in the Mycoplasma bacterium, wherein the first nucleotide arrangement comprises a nucleotide-encoded recombinase, the second nucleotide arrangement comprises two non-adjacent naturally occurring Mycoplasma sequences each having a minimum length of 5 nucleotides, optionally comprising a site-specific recombinase target site and/or a nucleotide-encoded selection marker, and the third nucleotide arrangement comprises a nucleotide-encoded nuclease.

In a further embodiment, the method comprises the introduction of a first, second, third, and fourth nucleotide arrangement as defined herein in the Mycoplasma bacterium, wherein the first nucleotide arrangement comprises a nucleotide-encoded recombinase, the second nucleotide arrangement comprises two non-adjacent naturally occurring Mycoplasma sequences each having a minimum length of 5 nucleotides, optionally comprising a site-specific recombinase target site and/or a nucleotide-encoded selection marker, the third nucleotide arrangement comprises a nucleotide-encoded nuclease, and the fourth nucleotide arrangement comprises at least one of the following elements: a single guide RNA, a tracrRNA, or a crRNA. In a further embodiment, the method comprises the introduction of a first, second, third, and fourth nucleotide arrangement as defined herein in the Mycoplasma bacterium, wherein the first nucleotide arrangement comprises a nucleotide-encoded recombinase, the second nucleotide arrangement comprises two non-adjacent naturally occurring Mycoplasma sequences each having a minimum length of 5 nucleotides, optionally comprising a site-specific recombinase target site and/or a nucleotide-encoded selection marker, the third nucleotide arrangement comprises a nucleotide-encoded nuclease, and the fourth nucleotide arrangement comprises a tracrRNA and a crRNA.

It is evident to a skilled person that the typical target-specific portion of a Cas9 gRNA, or the typical length of a target-specific Cas9 crRNA is about 20 nucleotides. It is additionally evident that gRNA sequences or crRNA sequences with aberrant lengths complexed with Cas9 may still display activity to an extent. Since it is known for the CRISPR/Cas9 system that base pairing with the target site tolerates mismatches in the distal portion of the target-specific sequence, shortening the crRNA or gRNA sequences by a limited amount of nucleotides, preferably no more than 5 nucleotides, may display increased specificity. Further shortening of the target specific sequence will inevitably result in a loss of specificity.

In a further embodiment, the method comprises the introduction of a second nucleotide arrangement as defined herein in a Mycoplasma bacterium, the second nucleotide arrangement comprising two non-adjacent naturally occurring Mycoplasma sequences each having a minimum length of 5 nucleotides, optionally comprising a site-specific recombinase target site and/or a nucleotide-encoded selection marker, after a first nucleotide arrangement as described herein has been introduced into the Mycoplasma bacterium, the first nucleotide arrangement comprising a recombinase. In a further embodiment, the method comprises the introduction of a third nucleotide arrangement as defined herein in a Mycoplasma bacterium, the third nucleotide arrangement comprising a nuclease, after a first and second nucleotide arrangement as described herein have been introduced into the Mycoplasma bacterium, the first nucleotide arrangement comprising a recombinase and the second nucleotide arrangement comprising a two non-adjacent naturally occurring Mycoplasma sequences each having a minimum length of 5 nucleotides, optionally comprising a site-specific recombinase target site and/or a nucleotide-encoded selection marker. In a further embodiment, the method comprises the introduction of a third and fourth nucleotide arrangement as defined herein in a Mycoplasma bacterium, the third nucleotide arrangement comprising a nuclease and the fourth nucleotide arrangement comprising at least one single guide RNA, or a tracrRNA, or at least one crRNA, after a first and second nucleotide arrangement as described herein have been introduced into the Mycoplasma bacterium, the first nucleotide arrangement comprising a recombinase and the second nucleotide arrangement comprising two non-adjacent naturally occurring Mycoplasma sequences each having a minimum length of 5 nucleotides, optionally comprising a site-specific recombinase target site and/or a nucleotide-encoded selection marker. In yet further embodiments, the method comprises the introduction of at least a second nucleotide arrangement, optionally further a third nucleotide, optionally further a fourth nucleotide in a Mycoplasma bacterium already comprising the first nucleotide arrangement. In yet further embodiments, the method comprises the introduction of at least a second nucleotide arrangement, optionally further a third nucleotide, optionally further a fourth nucleotide arrangement in a Mycoplasma bacterium already comprising the first nucleotide arrangement in its genomic sequence, wherein the second nucleotide arrangement comprises two non-adjacent naturally occurring Mycoplasma sequences each having a minimum length of 5 nucleotides, optionally comprising a site-specific recombinase target site and/or a nucleotide-encoded selection marker. In certain embodiments, the method further comprises selecting the Mycoplasma bacterium based on resistance to an antibiotic corresponding to an antibiotics resistance gene comprised in a nucleotide arrangement, preferably comprised in the second nucleotide arrangement as described herein. In certain embodiments, the method further comprises selection of the Mycoplasma bacterium based on expression of a fluorescent marker which is nucleotide-encoded in a nucleotide arrangement, preferably comprised in the second nucleotide arrangement. In certain embodiments, the fluorescent marker expressing Mycoplasma bacteria are separated from Mycoplasma bacteria not expressing the fluorescent marker by fluorescence flow cytometry. Methods for fluorescence cell sorting of bacteria have been described in the state of the art and are known to a skilled person (Nebe-von-Caron, Analysis of bacterial function by multi-colour fluorescence flow cytometry and single cell sorting. Journal of Microbiological Methods, 2000). In certain embodiments, the method comprises detection, preferably quantitative assessment of production of a heterologous nucleotide-encoded gene product comprised in a third nucleotide arrangement as described herein, wherein the third nucleotide arrangement preferably comprises a therapeutic protein (or peptide) or an immunogenic protein (or peptide).

In certain embodiments, the method comprises the introduction of a third nucleotide arrangement into Mycoplasma which induces cell death to any Mycoplasma bacterium not comprising a second nucleotide arrangement, wherein the third nucleotide arrangement comprises a nuclease and the second nucleotide arrangement comprises two nucleotide sequences of naturally occurring non-adjacent Mycoplasma sequences each having a minimum length of 5 nucleotides optionally comprising a site-specific recombinase target site and/or a nucleotide-encoded selection marker. In further embodiments wherein a third nucleotide arrangement is introduced into the Mycoplasma, said Mycoplasma comprises a first nucleotide arrangement comprising a nucleotide-encoded GP35 recombinase. In alternative embodiments wherein a third nucleotide arrangement is introduced into the Mycoplasma, said Mycoplasma comprises a nucleotide-encoded GP35 in its genomic sequence. In certain embodiments, the cell death is a direct consequence of DNA breaks induced by the activity of the nuclease comprised in the third nucleotide arrangement in the genome of the Mycoplasma bacterium. In further embodiments, the DNA breaks induced by activity of the nuclease comprised in the third nucleotide arrangement are positioned in a naturally occurring sequence within the Mycoplasma genome. In yet further embodiments, the DNA breaks induced by the activity of the nuclease comprised in the third nucleotide arrangement are positioned in a naturally occurring Mycoplasma genomic sequence which is unique for at least the Mycoplasma species used in the method.

In certain embodiments, the method is conducted on M pneumoniae. In certain embodiments, the M pneumoniae used in the herein described methods is publicly available. Suitable cell line providers are known to the skilled person and include both commercial and non-profit providers. In certain embodiments the M. pneumoniae used in the herein described methods is isolated from a subject diagnosed with pneumonia, preferably bacterial pneumonia. “Pneumonia” as used herein refers to an inflammatory condition of the lung affecting in particular the alveoli of the subject. The diagnosis of pneumonia is usually based on the assessment of physical signs, a chest radiograph, PCR-based methods, lung ultrasound, sputum cultures, or a combination thereof. Typical physical signs include but are not limited to low blood pressure, high heart rate, low oxygen saturation, increased respiratory rate, decreased chest expansion on the side affected by the pneumonia, bronchial breathing, crackling noises during inspiration, altered percussion of an affected lung, and increased vocal resonance. Methods and tools to investigate and/or verify the genetic identity of M pneumoniae have been described in the art and are therefore known to a skilled person (Xiao et al., Comparative genome analysis of Mycoplasma pneumoniae, BioMedCentral Genomics, 2015). In alternative embodiments, the method is conducted on a population of enriched Mycoplasma bacteria isolated from at least one subject diagnosed with pneumonia. In further embodiments, the method is conducted on a population of enriched Mycoplasma bacteria which may comprise different Mycoplasma species. In yet further embodiments wherein the method is conducted on a population of enriched Mycoplasma bacteria which may comprise different Mycoplasma species, the second nucleotide as described herein introduced to said Mycoplasma population may comprise at least one naturally occurring Mycoplasma sequence which is specific for M pneumoniae.

The term “diagnosed” as described herein indicates that a process of “diagnosing” has occurred and refers to the process or act of recognising, deciding on or concluding on a disease or condition in a subject on the basis of symptoms and signs and/or from results of various diagnostic procedures. It is common that a healthcare practitioner will simultaneously or near-simultaneously formulate a “prognostication” or “prognosis”. These terms are commonplace and well-understood in medical and clinical practice. It shall be understood that the phrase “a method for the diagnosis, prediction and/or prognosis” of a given disease or condition may also be interchanged with phrases such as “a method for diagnosing, predicting and/or prognosticating” of said disease or condition or “a method for making (or determining or establishing) the diagnosis, prediction and/or prognosis” of said disease or condition, or the like. A subject may be diagnosed as not having a disease despite displaying one or more conventional symptoms or signs reminiscent of such.

In certain embodiments the M pneumoniae used in the herein described methods is isolated from a subject predicted to have pneumonia, preferably bacterial pneumonia. In alternative embodiments the M pneumoniae used in the herein described methods is isolated from a subject predicted to have pneumonia, preferably bacterial pneumonia despite no manifestation of clinical symptoms. In alternative embodiments the M. pneumoniae used in the herein described methods is isolated from a subject predicted to show clinical manifestations of pneumonia symptoms.

“Predicting” or “prediction” generally refers to an advance declaration, indication or foretelling of a disease or condition in a subject not (yet) having said disease or condition. A prediction of a disease or condition in a subject may indicate a probability, chance or risk that the subject will develop said disease or condition within a certain time period or by a certain age. Said probability, chance or risk may be indicated inter alia as an absolute value, range or statistics, or may be indicated relative to a suitable control subject or subject population (e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population.

Also intended is the use of an oligonucleotide modification system according to any embodiment described herein for generating a genomically modified Mycoplasma bacterium. “Genomically modified” as used herein is indicative for an organism or a cell that comprises a genomic sequence aberrant from the genomic sequence of that organism or cell occurring in natural conditions. In certain embodiments, the oligonucleotide modification system used to generate a genomically modified Mycoplasma is a set of nucleotide arrangements, i.e. the system comprises a first nucleotide arrangement encoding a DNA binding molecule or protein, preferably a recombinase, and a second nucleotide arrangement a naturally occurring Mycoplasma sequence with a minimum length of 5 nucleotides. In certain embodiments, the oligonucleotide modification system used to generate a genomically modified Mycoplasma is a DNA binding molecule or protein, preferably a recombinase, and a nucleotide arrangement comprising a naturally occurring Mycoplasma sequence with a minimum length of 5 nucleotides. In alternative embodiments, the oligonucleotide modification system used to generate a genomically modified Mycoplasma is an RNA sequence encoding a DNA binding molecule or protein, preferably a recombinase, and a nucleotide arrangement comprising naturally occurring Mycoplasma sequences with a minimum length of 5 nucleotides. In certain embodiments, the genomic modification comprises at least one deletion of a naturally occurring sequence. In certain embodiments, the genomic modification comprises at least one insertion of a non-naturally occurring sequence. In a further embodiment the genomic modification comprises multiple insertions of non-naturally occurring sequences, preferably non-naturally occurring sequences comprising distinct sequences. In a further embodiment the genomic modification comprises a translocation of a naturally occurring sequence. In certain embodiments the genomic modification may induce a frame shift in an open reading frame of the organism's genome. In certain embodiments the genomic modification alters viability of the host organism or cell. In certain embodiments the genomic modification consists of a point mutation. In certain embodiments the genomic modification may be inducible by a compound. In further embodiments, the compound may be a small molecule. In further embodiments the compound may directly interact with at least one nucleotide arrangement as described herein. In alternative embodiments the compound may directly interact with at least one nucleotide encoded gene product comprised in at least one nucleotide arrangement as described herein, preferably wherein the gene product is a nuclease.

In certain embodiments, the use of an oligonucleotide modification system according to any embodiment described herein for generating a genomically modified Mycoplasma bacterium that is an attenuated Mycoplasma bacterium is envisaged. In preferred embodiments, the Mycoplasma bacterium is M. pneumoniae.

The term “attenuated” as described herein can be used interchangeably with terms such as “weakened” and “diminished”. The wording “attenuated strain” is commonly used in the art and refers to weakened disease agents, i.e. attenuated pathogens. An attenuated bacterium is a weakened, less vigorous, less virulent bacterium when compared to the traditionally occurring counterpart. Multiple vaccines against different diseases are based on inclusion of an attenuated strain of a bacterium or virus that is still capable of inducing an immune response and creating immunity but not causing illness. An attenuated Mycoplasma bacterium according to embodiments of the invention is indicative for a genomically modified Mycoplasma bacterium wherein expression of genes whereof the gene product is responsible for a certain degree of virulence or toxicity have been modified in order to diminish the adverse effect of said gene on an infected subject. In further embodiments, expression of a gene product responsible for a degree of toxicity is completely impeded by the genomic modification. In further embodiments, the promoter of the gene encoding the toxic gene product is inactivated by endogenous mutagenesis. In alternative further embodiments, a coding region, or exon, of a gene contributing to toxicity is mutagenized or removed by the genomic modification. In yet a further embodiment, a frame shift in a gene contributing to toxicity is induced by the genomic modification. In even further embodiments, a gene encoding a toxic or harmful gene product is replaced by a heterologous nucleotide-encoded gene product. In even further embodiments, the expression level of a toxic or harmful gene product is diminished by the genomic modification. In further embodiments, one or more fragments of a toxic or harmful gene are removed, whereby optionally the one or more fragments are removed without altering the reading frame and hence the modified gene product is still expressed.

In certain embodiments, a Mycoplasma strain as described herein is considered attenuated because the Mycoplasma bacterium has a reduced cytoadherence capacity to host cells when compared to the corresponding naturally occurring Mycoplasma strain. In further embodiments, the cytoadherence capacity of the attenuated Mycoplasma strain is reduced by at least 30%, at least 40%, at least 50%, at least 60%, at least 65%, at least 70%, at least 75%, at least 80%, at least 85%, at least 90% when compared to the corresponding naturally occurring Mycoplasma strain. In certain embodiments, a Mycoplasma strain as described herein is considered attenuated because the Mycoplasma bacterium has a reduced ability to fuse with host cells when compared to the corresponding naturally occurring Mycoplasma strain. In further embodiments, the ability to fuse with host cells of the attenuated Mycoplasma strain is reduced by at least 30%, at least 40%, at least 50%, at least 60%, at least 65%, at least 70%, at least 75%, at least 80%, at least 85%, at least 90% when compared to the corresponding naturally occurring Mycoplasma strain. In certain embodiments, a Mycoplasma strain as described herein is considered attenuated because the Mycoplasma bacterium has a reduced capacity to survive within host cells when compared to the corresponding naturally occurring Mycoplasma strain. In further embodiments, the capacity to survive within host cells of the attenuated Mycoplasma strain is reduced by at least 30%, at least 40%, at least 50%, at least 60%, at least 65%, at least 70%, at least 75%, at least 80%, at least 85%, at least 90% when compared to the corresponding naturally occurring Mycoplasma strain. In certain embodiments, a Mycoplasma strain as described herein is considered attenuated because the Mycoplasma bacterium has lower immunogenicity, i.e. the capacity to provoke an immune response when compared to the corresponding naturally occurring Mycoplasma strain. In further embodiments, the immunogenicity of the attenuated Mycoplasma strain is reduced by at least 30%, at least 40%, at least 50%, at least 60%, at least 65%, at least 70%, at least 75%, at least 80%, at least 85%, at least 90% when compared to the immunogenicity of the corresponding naturally occurring Mycoplasma strain. In certain embodiments, a Mycoplasma strain as described herein is considered attenuated because the Mycoplasma bacterium has limited organismal effects when compared to the corresponding naturally occurring Mycoplasma strain. In further embodiments, the organismal effects of the attenuated Mycoplasma strain is reduced by at least 30%, at least 40%, at least 50%, at least 60%, at least 65%, at least 70%, at least 75%, at least 80%, at least 85%, at least 90% when compared to the corresponding naturally occurring Mycoplasma strain. In preferred embodiments the host cell is a mammalian host cell, preferably a human host cell.

In certain embodiments the use of an oligonucleotide modification system according to any embodiment described herein is intended to generate an attenuated Mycoplasma bacterium that has a reduced toxicity of at least 30%, at least 35%, at least 40%, at least 45%, preferably at least 50%, at least 55%, at least 60%, at least 65%, at least 70%, preferably at least 75%, at least 80%, most preferably at least 85%, at least 90%, at least 95%, to a subject compared to a corresponding wild type Mycoplasma bacterium as based on the degree of necroptosis and apoptosis in human epithelial cells.

The term “necroptosis”, or “controlled necrosis” refers to a programmed form of necrosis that may be activated in response to the stimulation of death receptors by their cognate ligands in absence of caspase activity, the latter being an essential mediator of apoptosis. Morphological features of necroptosis closely resemble those of necrosis and include early plasma permeabilization, swelling of organelles, an expanded nuclear membrane, and chromatin condensation. “Apoptosis” refers to a programmed form of cell death. Morphological features of apoptosis include membrane blebbing, cell shrinkage, nuclear fragmentation, chromatin condensation, chromosomal DNA fragmentation, and mRNA decay.

Apoptosis may be initiated by the so-called intrinsic pathway initiated by cellular stress, or by the so-called extrinsic pathway initiated by extracellular signals. A skilled person is aware that the assessment of necroptosis may be performed in vivo or on a culture of cells.

In certain embodiments the use of an oligonucleotide modification system according to any embodiment described herein is intended to generate an attenuated Mycoplasma bacterium that has a reduced toxicity of at least 30%, at least 35%, at least 40%, at least 45%, preferably at least 50%, at least 55%, at least 60%, at least 65%, at least 70%, preferably at least 75%, at least 80%, most preferably at least 85%, at least 90%, at least 95%, to a subject compared to a corresponding wild type Mycoplasma bacterium as based on assessment of lung lesions in a (infected) host organism. In further embodiments the lung lesions are macro lung lesions, micro lung lesions, or a combination thereof. The term “lesions” as used herein is indicative for any damage or abnormal change in lung tissue of an infected organism.

In certain embodiments the use of an oligonucleotide modification system according to any embodiment described herein is intended to generate an attenuated Mycoplasma bacterium that has a reduced toxicity of at least 30%, at least 35%, at least 40%, at least 45%, preferably at least 50%, at least 55%, at least 60%, at least 65%, at least 70%, preferably at least 75%, at least 80%, most preferably at least 85%, at least 90%, at least 95%, to a subject compared to a corresponding wild type Mycoplasma bacterium as based on assessment of protein markers indicative for an immune response in said subject.

In certain embodiments the level or degree of toxicity is quantified by comparative analysis of the immune response to said attenuated Mycoplasma strain when compared to the corresponding naturally occurring Mycoplasma strain. Non-limiting examples of markers indicative for an immune response, or an absence of an immune response include Tumor Necrosis Factor alpha (TNF-α), Neutrophil chemokines (KC), Interferon gamma (INF-γ), Interleukin 1 beta (IL-1β), Interleukin 4 (IL-4), Interleukin 6 (IL-6), and Interleukin (IL18). An illustrative example for an in vitro assay suitable to evaluate the immune response is an enzyme-linked immunosorbent assay (ELISA). In certain embodiments the level or degree of attenuation is measured by comparative analysis of macroscopic and/or microscopic lesions. In certain embodiments the presence of Mycoplasma bacteria in host organisms is measured by performing a colony forming unit (CFU) assay or PCR reaction. CFU assays are commonly performed in the art and are therefore well known to a skilled person.

In certain embodiments the level or degree of toxicity is quantified by comparative assessment of pulmonary capacity and/or lung volume in a subject. Hence, in these embodiments, a reduced pulmonary capacity and/or lung volume when compared to a comparative control Mycoplasma strain is indicative for a increased level of toxicity. In certain embodiments the use of a set of nucleotides according to any embodiment described herein is intended to generate an attenuated Mycoplasma bacterium that has a reduced toxicity of at least 30%, at least 35%, at least 40%, at least 45%, preferably at least 50%, at least 55%, at least 60%, at least 65%, at least 70%, preferably at least 75%, at least 80%, most preferably at least 85%, at least 90%, at least 95%, to a subject compared to a corresponding wild type Mycoplasma bacterium as based on assessment of pulmonary capacity and/or lung volume in said subject.

The term “subject” refers to animals, preferably warm-blooded animals, more preferably vertebrates, and even more preferably mammals specifically including humans and non-human mammals, that have been the object of treatment, observation or experiment. The term “mammals”, or “mammalian subjects” refers to any animal classified as such and include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred subjects are human subjects including all genders and all age categories thereof.

In certain embodiments the use of an oligonucleotide modification system according to any embodiment described herein is intended to generate a genomically engineered Mycoplasma bacterium that expresses at least one heterologous protein. In certain embodiments the use of a set of nucleotides according to any embodiment described herein is intended to generate a genomically engineered Mycoplasma bacterium that expresses at least one heterologous protein that induces an immune response. In certain embodiments the use of a set of nucleotides according to any embodiment described herein is intended to generate a genomically engineered Mycoplasma bacterium that expresses at least one heterologous protein comprising multiple antigens. In certain embodiments the use of a set of nucleotides according to any embodiment described herein is intended to generate a genomically engineered Mycoplasma bacterium that expresses at least one in silico designed heterologous protein. In further embodiments the in silico designed heterologous protein comprises at least two antigens derived from at least two different naturally occurring proteins. In further embodiments the expressed heterologous protein is a fusion protein. In certain embodiments the heterologous protein further comprises a peptide or protein tag sequence. In further embodiments the genetically modified Mycoplasma bacterium secretes at least one heterologous protein. In further embodiments the genetically modified Mycoplasma bacterium secretes at least one heterologous protein which inhibits bacterial propagation. In certain embodiments the at least one heterologous protein secreted by the genomically modified Mycoplasma bacterium forms a multimeric protein. In further embodiments the protein secreted by the genomically modified Mycoplasma bacterium forms a homomultimeric protein. In certain embodiments the at least one heterologous protein is a transcription factor not naturally occurring in Mycoplasma. In further embodiments the genetically modified Mycoplasma bacterium secretes multiple heterologous proteins.

In further embodiments the secreted heterologous protein is an antibody or antibody like protein, a nanobody, a cytokine, or an enzyme. In alternative embodiments the genetically modified Mycoplasma bacterium displays at least one heterologous protein on its membrane. In certain embodiments the displayed heterologous protein is capable of inducing a huImoral immune response. In alternative embodiments the displayed heterologous protein is capable of inducing a cell-mediated immune response. In certain embodiment the displayed heterologous protein is a fusion protein. In certain embodiments the displayed heterologous protein is an antigen not naturally encoded by the genome of Mycoplasma. In certain embodiments the genetically modified Mycoplasma bacterium both expresses at least one first heterologous protein and displays at least one second heterologous protein. In further embodiments the at least one first secreted heterologous protein and the at least one second displayed heterologous protein are transcribed from a bicistronic or polycistronic nucleotide arrangement. In alternative embodiments the at least one first secreted heterologous protein and the at least one second displayed heterologous protein are transcribed from different nucleotide arrangements.

Further intended are Mycoplasma bacteria comprising a set of nucleotide arrangements as described by any embodiment herein, or an oligonucleotide modification system as described herein. In certain embodiments at least one nucleotide arrangement as described herein is present as an extrachromosomal genetic element in the Mycoplasma bacterium. In certain embodiments at least one nucleotide arrangement as described herein is present as an extrachromosomal genetic element in the Mycoplasma bacterium and at least a second nucleotide arrangement comprising a non-naturally occurring Mycoplasma sequence is comprised in the genome of said Mycoplasma bacterium. In certain embodiments where Mycoplasma bacteria comprising at least a one nucleotide arrangement are described by any embodiment, the at least one nucleotide arrangement is present in said Mycoplasma as part of a larger construct, preferably an expression construct, more preferably a double stranded DNA expression construct. In certain embodiments wherein the Mycoplasma bacteria comprise at least a one nucleotide arrangement as described by any embodiment, the at least one nucleotide arrangement is present as linear double stranded DNA. In certain embodiments wherein the Mycoplasma bacteria comprise at least a one nucleotide arrangement as described by any embodiment, the at least one nucleotide arrangement is present as linear single stranded DNA. In certain embodiments wherein the Mycoplasma bacteria comprise at least a one nucleotide arrangement as described by any embodiment, the at least one nucleotide arrangement is present as linear double stranded RNA. In certain embodiments wherein the Mycoplasma bacteria comprise at least one nucleotide arrangement as described by any embodiment, the at least one nucleotide arrangement is present as linear single stranded RNA. In Mycoplasma bacteria comprising more than one nucleotide arrangement as described herein, the different nucleotide arrangements may be present as different kinds of oligonucleotides, e.g. RNA and DNA, or single stranded DNA and double stranded DNA, or single stranded RNA and double stranded DNA. In certain embodiments a first and second nucleotide arrangements as described in any embodiment herein are present in the Mycoplasma bacterium as part of a single genetic construct. In certain embodiments the first and third nucleotide arrangements are present in the Mycoplasma bacterium as part of a single genetic construct. In certain embodiments, a third and fourth nucleotide arrangement as described herein are present in the Mycoplasma bacterium as part of a single genetic construct. Also intended are Mycoplasma bacteria obtained by a method described in any embodiment herein.

In certain embodiments, the Mycoplasma bacterium comprising a set of nucleotide sequences as 5 described herein or obtained by a method described herein, expresses a protein in a therapeutic effective amount. In certain embodiments, the Mycoplasma bacterium comprising a set of nucleotide sequences as described herein or obtained by a method described herein expresses a protein in a prophylactically effective amount.

The term “therapeutically effective dose” as used herein refers to an amount of a therapeutic protein or 10 therapeutic peptide as taught herein, that when administered brings about a positive therapeutic response with respect to treatment of a subject suffering from a disease, e.g. a patient having been selected (e.g. diagnosed) to have or a certain disease. In certain embodiments, the patient is diagnosed with pneumonia, preferably bacterial pneumonia. The term “prophylactically effective amount” refers to an amount of a gene product that inhibits or delays in a subject the onset of a disorder as being sought by a researcher, veterinarian, medical doctor or other clinician.

In certain embodiments the Mycoplasma bacterium comprises at least one nucleotide arrangement as described in any embodiment herein in its genomic sequence. In certain embodiments the Mycoplasma bacterium comprises at least one set of nucleotide arrangements as described in any embodiment herein in its genomic sequence. In certain embodiments, the Mycoplasma bacterium comprises a first nucleotide arrangement as described herein in its genomic sequence. In certain embodiments, the Mycoplasma bacterium comprises a second nucleotide arrangement as described herein in its genomic sequence. In certain embodiments, the Mycoplasma bacterium comprises a third nucleotide arrangement as described herein in its genomic sequence. In certain embodiments, the Mycoplasma bacterium comprises a first nucleotide arrangement and a second nucleotide arrangement as described herein in its genomic sequence. In certain embodiments, the Mycoplasma bacterium comprises a first nucleotide arrangement and a third nucleotide arrangement as described herein in its genomic sequence. In certain embodiments, the Mycoplasma bacterium comprises a first nucleotide arrangement, a second nucleotide arrangement and a third nucleotide arrangement as described herein in its genomic sequence. Methods to assess incorporation of a nucleotide arrangement as described herein in a bacterial or Mycoplasma genome are known to a person skilled in the art and include the non-limiting example of polymerase chain reaction (PCR)-based methods optionally coupled to fluorescence based-readouts, agarose gel-based readouts, or alternatively to restriction fragment length based readouts, targeted sequencing methods, whole genome sequencing methods, mismatch cleavage readouts, phenotypic readouts, and melting curve analyses (Germini et al., A comparison of techniques to evaluate the effectiveness of genome editing, Trends in Biotechnology, 2018).

In further embodiments, the Mycoplasma bacterium comprises at least a second nucleotide arrangement as described herein integrated in its genomic sequence. In further embodiments, the Mycoplasma bacterium comprises as single non-naturally occurring sequence (part of) a second nucleotide arrangement as described herein integrated in its genomic sequence, wherein the second nucleotide arrangement comprises at least a recombination site. In further embodiments, the Mycoplasma bacterium comprises as only non-naturally occurring sequence (part of) a second nucleotide arrangement as described herein integrated in its genomic sequence, wherein the second nucleotide arrangement comprises at least one nucleotide-encoded selection marker. In certain embodiments, only Mycoplasma bacteria comprising at least a second nucleotide arrangement as described herein in their genomic sequence are able to survive in the presence of a site-specific nuclease. In certain embodiments, incorporation of at least a second nucleotide as described herein in the genome of the Mycoplasma bacterium impedes binding of a nuclease to a sequence naturally comprised in the genome of Mycoplasma bacteria. In certain embodiments, incorporation of at least a second nucleotide arrangement infers a competitive advantage in terms of viability to the Mycoplasma bacterium when compared to Mycoplasma bacteria not comprising the at least one second nucleotide.

Further aspects of the invention relate to a kit of parts comprising 1) a DNA binding protein, preferably a recombinase, more preferably a GP35 recombinase protein, most preferably a GP35 recombinase having an amino acid sequence that is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the GP35 recombinase and 2) a second nucleotide arrangement comprising a naturally occurring Mycoplasma sequence with a minimum length of 5 nucleotides. In certain embodiments, the kit of parts comprises the recombinant recombinase and the second nucleotide arrangement in different storage vials. In certain embodiments, the recombinant recombinase is lyophilized. In certain embodiments, the second nucleotide arrangement is lyophilized. In certain embodiments, both the recombinant recombinase and the second nucleotide arrangement is lyophilized. In further embodiments the recombinant recombinase is a recombinant GP35 recombinase. In yet further embodiments, the recombinant GP35 recombinase is a GP35 recombinase having a amino acid sequence which is at least 65%, at least 70%, preferably at least 75%, at least 80%, more preferably at least 85%, at least 90%, most preferably 95%, 97%, 99% identical to the amino acid sequence of GP35 from Bacillus subtilis bacteriophage SPP1 as defined by SEQ ID NO: 1 based on the total length of the amino acid sequence of the recombinase. In certain embodiments, amino acid sequence of the GP35 recombinase has been optimized for improved activity in one or more Mycoplasma species, preferably M pneumoniae. In alternative embodiments, the kit of parts comprises a first nucleotide arrangement comprising a promoter or a functional variant or fragment thereof which is active in Mycoplasma bacteria operably linked to a nucleotide sequence encoding said DNA binding protein, preferably a recombinase, and a second nucleotide arrangement comprising a naturally occurring Mycoplasma sequence with a minimum length of 5 nucleotides. In yet alternative embodiments the kit of parts comprises an RNA sequence encoding said DNA binding protein, preferably a recombinase, and a second nucleotide arrangement comprising a naturally occurring Mycoplasma sequence with a minimum length of 5 nucleotides. A skilled person readily appreciates that the GP35 recombinase protein may be recombinant GP35 recombinase, purified GP35 recombinase, or a combination thereof.

Alternative kit of parts that are envisaged by the present disclosure are kit of parts that comprise 1) a first nucleotide arrangement comprising a promoter or a functional variant or fragment thereof which is active in Mycoplasma bacteria, operably linked to a nucleotide sequence encoding a GP35 recombinase, and/or an RNA sequence encoding said DNA binding protein, and 2) a second nucleotide arrangement comprising a naturally occurring Mycoplasma sequence with a minimum length of 5 nucleotides. As evident to a skilled person, the contents of the kits of parts may be provided as lyophilized components, wherein the kit optionally comprises at least one resuspension buffer for one or more of the kit of part components. Alternatively, the contents of the kit of parts may be dissolved at a stock concentration.

Evidently, any kit of parts may be accompanied by instructions on the use thereof, documents regarding safety, documents concerning quality assurance and any other information that is commonly provided in kit of parts.

In certain embodiments, the kit of parts further comprises a third nucleotide arrangement as described in any instance throughout this description or a recombinant nuclease. In certain embodiments, the kit of parts further comprises a third nucleotide arrangement as described in any instance throughout this description and a recombinant nuclease. In certain embodiments, the kit of parts further comprises a single guide RNA. In alternative embodiments, the kit of parts further comprises a tracrRNA. In further embodiments, the kit of parts further comprises a tracrRNA and at least one crRNA. In certain embodiments wherein the recombinant nuclease is a Cas protein, the kit of parts comprises the Cas protein complexed with a single guide RNA. In alternative embodiments wherein the recombinant nuclease is a Cas protein, the kit of parts comprises the Cas protein complexed with both the tracrRNA and the crRNA. In certain embodiments wherein the kit of parts includes a nucleotide arrangement comprising at least one site-specific recombinase site, said kit of parts further comprises a second site specific recombinase specifically targeting said at least one site-specific recombinase site. In certain embodiments the kit of parts further comprises a viable Mycoplasma bacterium. In further embodiments, the viable Mycoplasma bacterium is lyophilized. In alternative embodiments, the viable Mycoplasma bacterium is provided in a frozen condition.

A further aspect of the invention is directed to a method of altering the genome of a Mycoplasma bacterium wherein the method comprises introducing the components of any kit of parts as described herein into the Mycoplasma bacterium, preferably M pneumoniae. In certain embodiments, the method comprises the introduction of all kit of parts components at the same time point. In alternative embodiments, the method comprises the introduction of at least two kit of parts components at different time points. In certain embodiments, the method comprises the repeated introduction of at least one kit of parts components.

Additionally, the disclosure provides the following statements:
Statement 1. An oligonucleotide modification system comprising:
   a DNA binding protein or a first nucleotide arrangement comprising a promoter or a functional variant or fragment thereof which is active in Mycoplasma bacteria, operably linked to a nucleotide sequence encoding said DNA binding protein, or an RNA sequence encoding said DNA binding protein, and
   a second nucleotide arrangement comprising a naturally occurring Mycoplasma sequence with a minimum length of 5 nucleotides.
Statement 2. The oligonucleotide modification system according to statement 1, wherein the naturally occurring Mycoplasma sequence is comprised as two non-adjacent nucleotide sequences in the second nucleotide arrangement, each having a minimum length of 5 nucleotides.
Statement 3. The oligonucleotide modification system according to statement 2, wherein the two non-adjacent nucleotide sequences in the second nucleotide arrangement that are naturally occurring Mycoplasma sequences are separated from each other by a nucleotide sequence not-naturally occurring in Mycoplasma.
Statement 4. The oligonucleotide modification system according to statement 3, wherein the nucleotide sequence not-naturally occurring in Mycoplasma comprises at least a restriction site, a site-specific recombinase target site or a nucleotide-encoded selection marker or any combination thereof, wherein preferably the site-specific recombinase target site is a lox site.
Statement 5. The oligonucleotide modification system according to any one of statements 1 to 4, wherein the DNA binding protein comprised in the first nucleotide arrangement is a recombinase, more preferably a GP35 recombinase, most preferably a GP35 recombinase having a nucleotide sequence encoding a protein which is at least 65% identical, preferably at least 75% identical, more preferably at least 85% identical, most preferably at least 95% identical to the amino acid sequence of GP35 from Bacillus subtilis bacteriophage SPP1 as defined by SEQ ID NO: 1 based on the total length of the amino acid sequence of the recombinase.
Statement 6. The oligonucleotide modification system according to any one of statements 1 to 5, further comprising a third nucleotide arrangement comprising a promoter or a functional variant or fragment thereof which is active in Mycoplasma bacteria, operably linked to a heterologous nucleotide-encoded gene product, preferably wherein the heterologous nucleotide-encoded gene product comprises a therapeutic protein or an immunogenic protein.
Statement 7. The oligonucleotide modification system according to statement 6, wherein the heterologous nucleotide-encoded gene product further comprises an exposure signal sequence or secretion signal sequence.
Statement 8. The oligonucleotide modification system according to any one of statements 1 to 5, further comprising a third nucleotide arrangement comprising a promoter or functional variant or fragment thereof which is active in Mycoplasma bacteria, operably linked to a nucleotide-encoded nuclease or a nucleotide-encoded recombinase.
Statement 9. The oligonucleotide modification system according to statement 8, wherein the nucleotide-encoded nuclease comprised in the third nucleotide arrangement is an endonuclease, preferably an endonuclease selected from the group comprising of restriction enzymes, meganucleases, zinc-finger nucleases (ZFNs), transcription-activator like effector nucleases (TALENs), and CRISPR associated (Cas)-based nucleases.
Statement 10. The oligonucleotide modification system according to statement 9, wherein the Cas-based nuclease is a type II Cas nuclease, preferably a Cas9 nuclease having a nucleotide sequence encoding a protein which is at least 65% identical, preferably at least 75% identical, more preferably at least 85% identical, most preferably at least 95% identical to the amino acid sequence of Cas9 from Streptococcus pyogenes as defined by SEQ ID NO: 2 based on the total length of the amino acid sequence of the Cas9 or functional variant thereof.
Statement 11. The oligonucleotide modification system according to statement 10, further comprising a fourth nucleotide arrangement comprising at least one single guide RNA sequence, or at least one crRNA sequence and a tracrRNA sequence, capable of base pairing with a naturally occurring sequence in a Mycoplasma genome.
Statement 12. The oligonucleotide modification system according to any one of statements 1 to 11, wherein at least one nucleotide arrangement comprises a promoter with a nucleotide sequence of at least 65% identity, preferably at least 75% identity, more preferably at least 85% identity, most preferably at least 95% identity to the nucleotide sequence of SEQ ID NO: 3.
Statement 13. The oligonucleotide modification system according to any one of statements 1 to 12, wherein at least one of the nucleotide arrangements further comprises a regulatory sequence capable of modulating transcription, preferably wherein the regulatory sequence is a riboswitch.
Statement 14. Use of a GP35 recombinase for altering the genomic sequence of a Mycoplasma bacterium.
Statement 15. A method of altering the genome of a Mycoplasma bacterium, comprising introducing the oligonucleotide modification system according to any one of statements I to 13, or introducing at least one of the nucleotide arrangements as defined in any one of statements 1 to 13 into a Mycoplasma bacterium.
Statement 16. Use of an oligonucleotide modification system according to any one of statements 1 to 13, or the method according to statement 15 for generating a genomically modified Mycoplasma bacterium.
Statement 17. A Mycoplasma bacterium comprising the oligonucleotide modification system according to any one of statements 1 to 13, or obtained by the method according to statement 15.

The herein disclosed aspects, embodiments, and statements of the invention are further supported by the following non-limiting examples.

### Examples

### 1. Identification of Effector Proteins for Use in an Oligonucleotide Modification System for Mycoplasma.

In order to overcome the lack of genome-editing tools available forM. pneumoniae, we aimed to develop an oligo recombineering system functional for this strain. This technology relies on two consecutive events. The first step is the homology-driven positioning of oligonucleotides at the lagging strand of the replication fork, a process that in bacteria can be boosted by phage-derived ssDNA recombinases (Datta et al., Identification and analysis of recombineering functions from Gram-negative and Gram-positive bacteria and their phages, Proceedings of the National Academy of Sciences of the United States of America, 2008). Subsequently, the arranged oligonucleotide is incorporated into the newly-synthesized chromosomal copy as an Okazaki fragment, thereby mediating the introduction of the intended modifications in the genome. The apparent simplicity of this process, together with the universal conservation of the replication mechanism, might led one to assume that oligo recombineering is a broadly portable technology capable of editing genomes independently of the host recombination machinery. However, phage-derived recombinases do not maintain their efficient performance across different bacterial genera, suggesting some sort of dependence on host machinery, as was recently published by Sun and colleagues, who observed a pronounced decrease in recombineering efficiencies when testing recombinases expressed by non-native B. subtilis phages (Sun et al., A high-efficiency recombineering system with PCR-based ssDNA in Bacillus subtilis mediated by the native phage recombinase GP35, Applied Microbiology and Biotechnology, 2015). Indeed, it seems that the recombineering frequency obtained depends on the phylogenetic distance between the native host of the phage and the bacteria being engineered (Wang et al., Programming cells by multiplex genome engineering and accelerated evolution, Nature, 2009). Prompted by these observations, we decided to survey the Mycoplasma pan-genome as well as their associated phages for the presence of orthologous proteins to RecB and RecT. These two proteins from the lambda and Rac phages, respectively, are the best-characterized and most frequently employed phage-derived ssDNA recombinases in oligo recombineering protocols (Ellis et al., High efficiency mutagenesis, repair, and engineering of chromosomal DNA using single-stranded oligonucleotides, Proceedings of the National Academy of Sciences of the United States of America, 2001).

We found three RecT-like proteins coded by the genomes of Spiroplasma melliferum, Spiroplasma citri, and Spiroplasma poulsonii, which we renamed RecTₛₘ, RecT_{sc}, and RecTₛₚ, respectively. On the other hand, our search for Mycoplasma RecB orthologues did not produce any relevant candidate and lead us to select the more distant GP35 as a RecB-like protein to include in our screening. This protein was recently reported to be an efficient phage-derived recombinase for performing genome editing in Bacillus subtilis (Sun et al., A high-efficiency recombineering system with PCR-based ssDNA in Bacillus subtilis mediated by the native phage recombinase GP35, Applied Microbiology and Biotechnology, 2015). Despite the notion in said publication that recombinases expressed by native phage are favorable for obtaining efficient recombineering in the corresponding bacterium, the recombineering efficiency of GP35 in Mycoplasma (a non-corresponding bacterium) was assessed.

### 2. Establishment of an Oligonucleotide Modification System for Mycoplasma.

Generation of aM. Pneumoniae Test Strain Comprising aMutCm+1 Recombineering Sensor

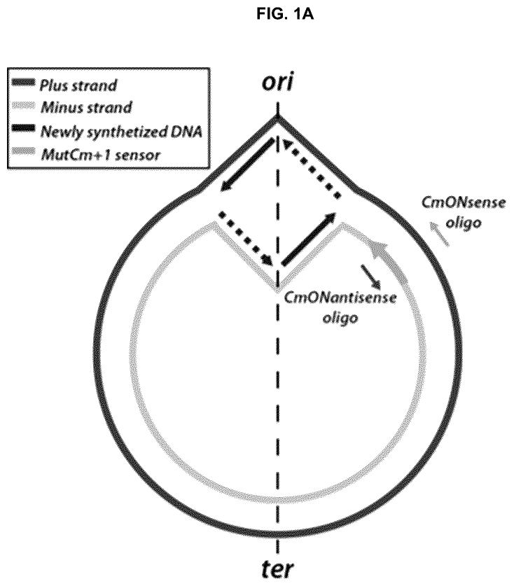

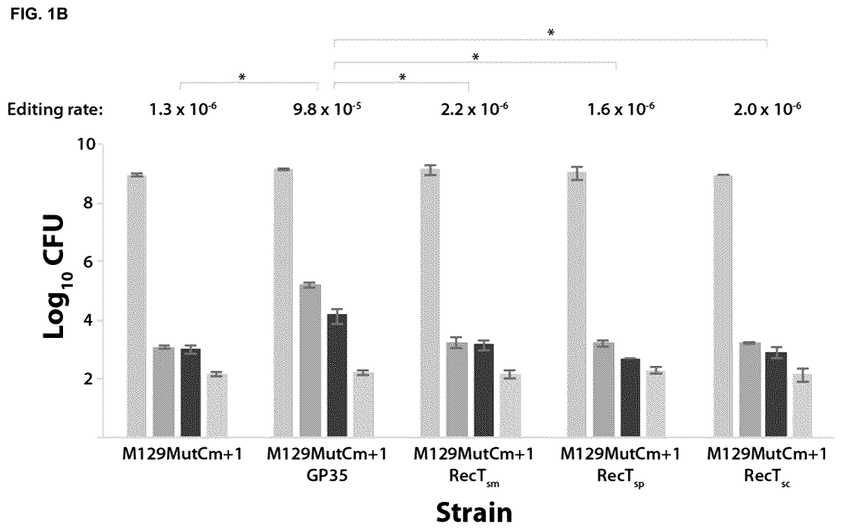

The ability of the different proteins to introduce changes to the M. pneumoniae genome by catalyzing oligo recombineering was experimentally monitored with a recombineering sensor termed MutCm+1. This sensor is based on a chloramphenicol acetyltransferase gene (cat) whose protein product confers resistance to chloramphenicol (Cm). Nevertheless, the cat coding sequence present in the MutCm+1 sensor is frame-shifted by the addition of a single nucleotide at position 310, rendering a protein product unable to confer resistance to the antibiotic. In order to correct the MutCm+1 sensor, we designed two different oligonucleotides termed CmONsense (T*T*G*T*TACACCGTTTTCCATGAGCAAACTGAAACGTTTTCATCGCTCTGGAGTGAATA CCACGACGATTTCCGGCAGTTTC; SEQ ID NO: 53) and CmONantisense (G*A*A*A*CTGCCGGAAATCGTCGTGGTATTCACTCCAGAGCGATGAAAACGTTTCAGTTT GCTCATGGAAAACGGTGTAACAA; SEQ ID NO: 54), following the rules reported in a screening of optimized design criteria for recombineering oligonucleotides (Ricaurte et al., A standardized workflow for surveying recombinases expands bacterial genome-editing capabilities, Microbial biotechnology, 2018). Asterisks shown at the 5′ end of the oligonucleotide sequences are indicative for phosphorothioate bonds. Both oligonucleotides have the exact same sequence present in the region surrounding the frame-shift included in the sensor, except for the extra nucleotide. However, whereas CmONsense has the same orientation as the frame-shifted cat gene, the CmONantisense oligonucleotide is antisense to the sequence of the cat gene. In principle, any of the oligonucleotides could mediate the deletion of the frame-shifted nucleotide and the consequent activation of the cat gene. However, as oligonucleotides are incorporated as Okazaki fragments into the newly synthesized chromosome, those targeting the lagging strand produce a substantially higher editing rate than those binding the leading strand of the replication fork. Thus, determining the location of the MutCm+1 sensor within the M pneumoniae genome was essential for our screen, as synthesis of a DNA strand as either leading or lagging depends on its chromosomal location with respect to the origin of replication. To this end, the MutCm+1 sensor was cloned into a transposon vector and transformed into M. pneumoniae WT cells, to generate a strain termed M129MutCm+1 (FIG. 1A). After clone isolation, we used an arbitrary PCR (A-PCR) protocol to locate the transposon insertion at genome position 60115 (MPN049 locus) of the minus strand (Welsh and McClelland, Fingerprinting genomes using PCR with arbitrary primers, Nucleic acids research, 1990). Thus, the CmONsense oligonucleotide would be the one targeting the lagging strand at this location and should yield a higher number of edited cells.

### Bacterial Strains and Culture Conditions

All the M. pneumoniae strains generated in this work are described in Table S5 and are derived from wild type strain M129-B7 (ATTC 29342, subtype 1, broth passage no. 35). All the strains were grown in Hayflick modified medium at 37° C. under 5% CO2 in tissue culture flasks (Coming). Hayflick broth was supplemented with tetracycline (2 μg ml⁻¹), puromycin (3 μg ml⁻¹) or chloramphenicol (20 μg ml⁻¹) for selection of cells as needed, or with anhydrotetracycline at the indicated concentrations for inducing Cas9 expression. When growth on plate was required Hayflick broth was supplemented with 0.8% bacto agar (Difco).

For cloning purposes, the E. coli NEB© 5-alpha High Efficiency strain (New England Biolabs) was grown at 37° C. in LB broth or on LB agar plates supplemented with ampicillin (100 μg ml−1).

### Plasmids

All the plasmids generated in this work were assembled using the Gibson cloning method (Gibson, Enzymatic assembly of overlapping DNA fragments). When required, DNA fragments were obtained through gene synthesis by a commercial supplier (IDT DNA Technologies). Oligonucleotides were synthesized by Sigma-Aldrich. Gene amplifications were carried out with Phusion DNA polymerase (Thermo Fisher Scientific) The correct assembly of all the plasmids was verified by Sanger sequencing (GATC biotech).

BLAST Search of Orthologues and CLUSTALW Multiple Sequence Alignment Mycoplasma orthologues of E. coli-derived proteins RecT (P33228) and RecB (P03698) were searched using BLASTp (protein-protein BLAST). Parameters of the search were restricted to Mycoplasmas and walled relatives (taxid: 31969), Mycoplasma phage phiMFV1 (taxid: 280702), Mycoplasma phage MAV1 (taxid: 75590) and Mycoplasma phage P1 (taxid: 35238). RecT-associated positive hits of this search, here renamed as RecTsm (WP_004028097.1), RecTsp (WP_127093247.1) and RecTsc (CAK99285.1) were later aligned using CLUSTALW software taking the native E. coli-derived RecT protein as a reference.

### Transposon Insertion Localization by A-PCR

Cultures of the clones of interest were grown on 25 cm² flasks until reaching confluence. The adherent layer of cells was washed three times with PBS and scrapped off in 300 μl of this buffer. This cell solution was treated with MasterPure DNA Purification Kit (Epicentre) following the manufacturer's instructions to isolate genomic DNA.

The A-PCR protocol followed is a variant of the one previously described (Das et al. An improved arbitrary primed PCR method for rapid characterization of transposon insertion sites, Journal of Microbiological Methods, 2005) in order to adapt it to M pneumoniae genome composition. Specifically, we modified the 3′ end of the arbitrary oligo in order to mimic the most frequent pentanucleotide sequence in the M. pneumoniae genome that ends in a “GC clump”. The script for finding the most frequent pentanucleotides for a given genome and sort them by the number of hits is freely available at: https://github.com/jdelgadoblanco/pentanucleotides.git.

### Screening of Edited Clones

96 well plates were prepared as following. All the perimeter wells were filled with 200 μl of Hayflick medium as colour reference. Then, the “inoculation wells” were filled with 200 μl of Hayflick medium, the “non-selective wells” with 150 μl of Hayflick medium, and the “Cm-selective wells” with 150 μl of Hayflick medium supplemented with Cm at 1.25× concentration. Colonies were picked from the plates of interest and transferred into the inoculation wells by pipetting up and down several times. Subsequently, separate aliquots of 50 μl were transferred from the inoculation well to both the “non-selective well” and the “cm-selective well”. Plates were incubated at 37° C. under 5% CO2 for seven days before taking pictures of them with ImageScannerIII (Epson).

### Testing of Candidate Effector Proteins in the MutCm+1I Strain

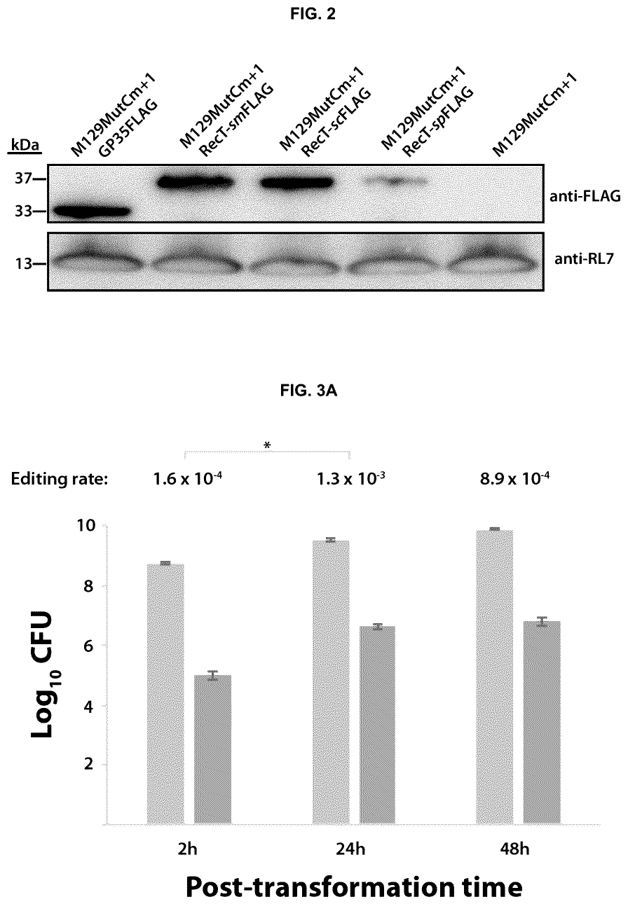

Four different strains, all containing the MutCm+1 sensor and the different recombinases found in our orthologue search (e.g., GP35, RecT-sm, RecT-sc, and RecT-sp), were subjected to a mock transformation or to transformations with the editing oligos CmONsense and CmONantisense. In addition, a control strain not expressing any recombinase was included in this screening (FIG. 2). Two hours post-transformation, serial dilutions of cells were seeded on non-selective plates to calculate the total amount of cells, or on Cm-supplemented plates to determine the amount of edited cells for each condition. These values allowed for calculation of the editing rate (edited cells/total cells) for each recombinase and condition.

M pneumoniae Transformations

Transformations were performed as described previously with few modifications (Hedreyda, et al., Transformation of M pneumoniae with Tn4001 by electroporation. Plasmid, 1993). Briefly, M pneumoniae cultures were grown to late-exponential phase in 75 cm2 tissue cultures flasks. The adherent layer of M pneumoniae cells was washed three times with chilled electroporation buffer, scrapped off and resuspended in 500 μl of this buffer at a concentration of approximately 10¹⁰ cells ml⁻¹. Next, this cell suspension was passed 10 times through a 25-gauge (G25) syringe needle, and 50 μl aliquots were mixed with the desired DNA molecules to transform. For transposon vector transformations 2 μg of DNA were added to the mix whereas for oligo transformations the volumes employed were 1, 5 or 10 μl of a 100 μM stock, corresponding to 0.1, 0.5 or 1 nmol, respectively. The mixture of DNA and cells was adjusted to a final volume of 80 μl and subsequently transferred into 0.1 cm electrocuvettes, letting it sit for 15 minutes on ice before being electroporated in a BIG-RAD Gene Pulser Xcell apparatus. The settings employed were 1250 V/25 μF/100Ω. After the pulse, cells were incubated on ice for 15 minutes and subsequently harvested by adding 420 μl of Hayflick medium into the cuvette. In the case of transposon vector transformations, cells were allowed to recover at 37° C. for two hours before inoculating one fifth of the transformation volume into a 25 cm² flask filled with 5 ml of Hayflick medium supplemented with the appropriate antibiotic. In those cases wherein individual clones of the transformation were required, after the two hours of recovering time, serial dilutions were seeded on plates and individual clones were picked and clonally expanded. In the case of oligo transformations wherein several pulses were performed, cells were allowed to recover 3 minutes on ice between the pulses. Afterwards, the total volume of the transformation was directly inoculated into T75 flasks filled 25 ml of Hayflick medium.

### Editing Rate Determination

M pneumoniae cells carrying one of the different recombineering sensors generated in this work and a second transposon harbouring one of the recombinases screened and optionally also the enhanced and inducibleCas9 (eiCas9) system were transformed with an editing oligo. At the indicated post-transformation time, the transformed cells were scraped off from the flask and into 500 μl of Hayflick medium. Subsequently, 10-fold serial dilutions were performed (from −1 to −8). Dilutions were made in a total volume of 100 μl and 10 μl of each dilution were spotted onto Hayflick 0.8% bacto agar plates supplemented with chloramphenicol and/or anhydrotetracycline when appropriate. Thus, the detection limit of these experiments is 500 CFU. When the number of cells obtained for a given condition was below this detection limit, the maximum possible number of cells (i.e. 499 CFU) was considered for statistical analyses. The editing rate is defined as the number of cells resistant to chloramphenicol divided by the total number of cells obtained for each condition and is depicted in FIG. 1B. Paired t-test analysis of the editing rates obtained in the three biological replicas conducted for each condition was performed using GraphPad QuickCalcs software. An asterisk (*) was included in FIG. 1B when the difference in the editing rate for two given conditions was found to be statistically significant (p<0.05).

### Western Blot Analysis

The strains to be analysed were grown on 25 cm² flasks until reaching confluence. The adherent layer of cells was washed twice with PBS and scraped off in 500 μl of this buffer. The cell solution was centrifuged (12000× g 5 min) and the resulting pellet lysed in 150 of lysis buffer (SDS 4%, Hepes 100 mM). Subsequently, Mycoplasma cell lysates were quantified using the Pierce BCA Protein Assay Kit and 10 μg of cell extracts were subjected to electrophoresis through NuPAGE 4-12% Bis-Tris pre-cast polyacrylamide gels (Invitrogen). Next, proteins were transferred onto nitrocellulose membranes using an iBlot dry blotting system (Invitrogen). Novex Sharp Pre-stained Proteins Standards allowed cutting the membrane into two individual pieces to process individually. Both membrane pieces were blocked with 5% skim milk (Sigma) in Tris-buffered saline (TBS) solution, containing 0.1% Tween 20 (TBST). Upper membrane piece (containing proteins above 20 kDa) was probed with monoclonal anti-FLAG M2 (Sigma) as primary antibody (1:5000) and anti-mouse IgG (1:10000) conjugated to horseradish peroxidase (Sigma) as secondary antibody. The lower membrane piece (containing proteins below 20 kDa) was probed with anti-RL7 polyclonal serum as primary antibody (1:1000) and anti-rabbit IgG (1:10000) coupled to horseradish peroxidase (Sigma) as secondary antibody. Blots were developed with the Supersignal West Femto Chemiluminescent Substrate Detection Kit (ThermoScientific) and the resulting signals were detected in a LAS-3000 Imaging System (Fujifilm).

### CONCLUSIONS

For all strains assessed, mock transformations rendered a low proportion of cells that are Cm-resistant (FIG. 1B). The small amount of cells observed in all the cases (i.e. ˜2×10²) might represent spontaneous mutants resistant to Cm or mutants that might arise as a consequence of poor Cm selective pressure when highly-concentrated dilutions are spotted. In any case, this frequency of Cm-resistant cells should be considered as a background signal of our screening, as its occurrence is not mediated by a recombineering phenomenon.

The amount of Cm-resistant cells increased in all the strains that were transformed with the CmONantisense oligonucleotide (FIG. 1B). However, the amount of edited cells barely overcome the background signal of the screening (i.e. ˜9×10²vs ˜2×10²), except for the strain expressing the GP35 recombinase, for which we detected an increase of almost two-orders of magnitude in the amount of Cm-resistant cells as compared to the background signal (1.6×10⁴ vs ˜2×10², respectively).

Moreover, the difference between the GP35-expressing strain and all others was further increased when CmONsense was the transforming oligonucleotide (FIG. 1B). In this scenario, the amount of edited (Cm-resistant) cells was 1.6×10⁵ for the strain expressing GP35, but only slightly higher than background signal (i.e. ˜2×10³ vs ˜2×10²) for all other strains.

Altogether, these results suggest that none of the RecT-like recombinases are functional in M. pneumoniae, even though the expression levels of at least RecT-sm and RecT-sc were similar to the those in the strain expressing the GP35 recombinase (FIG. 2). Surprisingly, RecT-sm and RecT-sc are annotated as a RecT family protein and a putative RecT protein, respectively. Although it cannot be ruled out that these proteins might behave as actual recombinases in their native organisms, it seems that they could be carrying out alternative functions, despite showing a moderate sequence similarity with RecT proteins. In contrast, we found that GP35 is a functional protein that performs oligonucleotide recombineering in M. pneumoniae, with an editing efficiency reaching 9.8×10⁻⁵. This result appears to contradict the finding that was reported by Sun et al. with respect to the inverse correlation between the phylogenetic distance between the native host of the phage and the bacteria being engineered

3. Optimization of GP35-Mediated Oligo Recombineering in M. pneumoniae.

Our screening highlighted GP35 as the first reported recombinase capable of mediating oligonucleotide recombineering in a Mycoplasma strain such as M pneumoniae. Yet, the editing efficiencies obtained were still far from those reported for other pairs of recombinase-microorganism. For instance, oligo recombineering in E. coli by expressing RecB protein mediates 1 bp gene editing with efficiencies up to 2×10⁻¹, whereas the same type of modification is obtained at frequencies of 1.8×10⁻³ for Pseudomonas putida expressing Rec2 protein or at 2.5×10⁻³ for Staphylococcus aureus expressing EF2132 protein. Consequently, we attempted to increase the efficiency by empirically improving some parameters of the recombineering protocol.

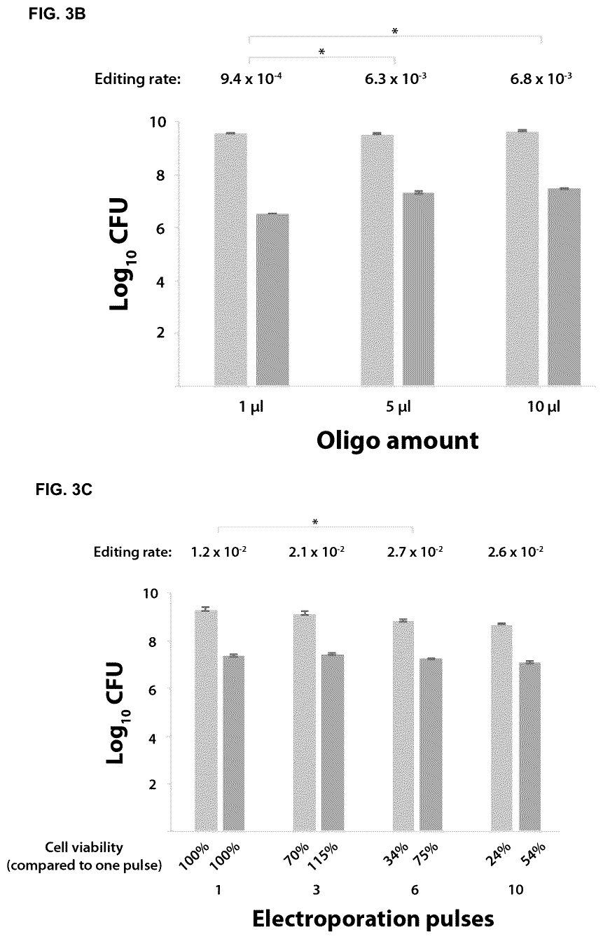

As a starting point, we reasoned that if GP35 protein catalyzes the incorporation of the oligonucleotide at the replication fork, a permissive window of time should be considered to perform its task. For a slow-dividing microorganism such as M. pneumoniae having a doubling time of approximately eight hours, the two hours interval between transformation and plate seeding employed in the initial screening might be insufficient to ensure that the replication fork has passed at least once across the desired locus in all the cells of the population. To test this hypothesis, the GP35-expressing strain carrying the MutCm+1 sensor was transformed with the CmONsense oligonucleotide, whereafter the cells were grown under non-selective conditions for either 2, 24, or 48 hours. following culturing, cells were seeded on plates to determine the amount of total and edited cells, as well as the edition rate for each condition (FIG. 3A).

In line with our hypothesis, when cells that received the oligonucleotide were grown for 24 hours before plating, the editing efficiency increased to 1.3×10⁻³. This improvement of one-order of magnitude reflects the fact that during the time window between 2-hours and 24-hours post-transformation, the total number of cells rose almost six times (5.3×10⁸ vs. 3.1×10⁹, respectively), whereas the numbers of edited cells increased ˜43 times (9.2×10⁴ vs. 4×10⁶, respectively). This further corroborates that the GP35-oligo editing mechanism is linked to the replication machinery. A similar editing efficiency was observed when 24— and 48-hour post transformation time points are compared (1.3×10⁻³ vs 8.9×10⁴, respectively). Firstly, this implies that longer times do not cause a further improvement of the editing efficiency, and secondly that after 24 hours, most oligonucleotides were either degraded and/or already incorporated into the chromosome.

To further increase the transformation efficiency, we examined the contribution of the amount of recombination substrate. We thus compared the editing rates obtained when 1 μl, 5 μl, or 10 μl of CmONsense oligonucleotide stock (100 μM) was added to the transformation mixture (note that 1 μl was used in the previous experiments detailed above). After transformation, cells were grown for 24 hours prior to seeding (FIG. 3B). Increasing the oligonucleotide amount from 1 μl to 5 μl enhanced the editing rate approximately seven times (9.4×10⁴ vs. 6.3×10⁹, respectively), whereas using 10 μl did not significantly improve this rate (giving 6.8×10⁻³). Thus, we conclude that using 5 μl of the editing oligonucleotide is sufficient to saturate the recombineering process.

Finally, we explored whether several electroporation pulses could improve editing efficiency. Though rarely employed, a similar strategy has been applied for plasmid transformation in Agrobacterium tumefaciens and led to an increase of the number of transformed cells by 2—to 5 times, depending on the particular strain. Thus, for this screening, M129 TcMutCm+1 cells were transformed with 5 μl of CmONsense oligonucleotide and subjected to 1, 3, 6, or 10 electroporation pulses. At 24 hours post-transformation, cells were seeded to determine the editing rate for each condition (FIG. 3C).

Essentially, we observed a general trend of compromised viability with increasing number of pulses: relative to the cells that survived a single pulse, only ˜30% of cells remained viable after 6 or 10 pulses. However, the number of edited cells increased with the number of pulses, at least until the viability of the total resulting population started to be severely compromised. Altogether, this screening pointed out that 6 electroporation pulses is an appropriate trade-off point between an increased number of edited cells and total cell viability. Indeed, there is a 2.3-fold increase in the editing rate obtained with 6 electroporation pulses as compared to one pulse (2.7×10⁻² vs 1.2×10⁻², respectively). In sum, after a limited screening of a series of parameters that could affect the recombineering process, we increased the editing rate 165 times (from 1.6×10⁻⁴ to 2.7×10⁻²). Indeed, after optimization, the frequency of incorporation of a 1-bp deletion in M. pneumoniae outperformed that reported for other bacteria, such as S. aureus or P. putida, by one order of magnitude. Further factors might also improve the efficiency of this process. By means of illustration and not limitation, extending the length of the oligonucleotide to increase complementarity with its target sequence might facilitate its incorporation into the replication fork. However, a longer length additionally facilitates the formation of secondary structures in the oligonucleotide, which may hinder its accessibility to the cell in the first place, as well as impede its introduction into the replication fork. For this reason, our oligonucleotide design was based on the rules followed by recombineering protocols for other bacteria (i.e. 80-90 bp in length, central position of the mismatch, and 5′-end protection). Nonetheless, we cannot exclude that recombineering in M. pneumoniae might be boosted by extended oligo-target complementarity.

### 4. Further Characterisation of Recombineering Efficiencies.

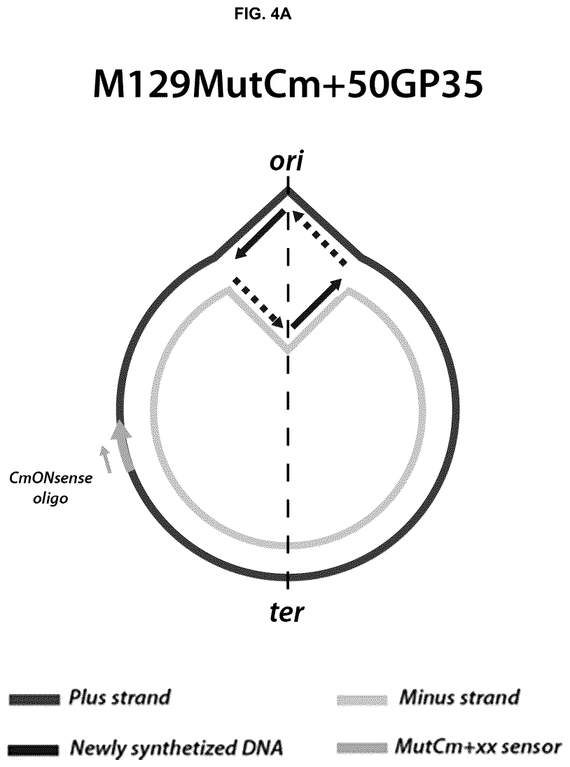

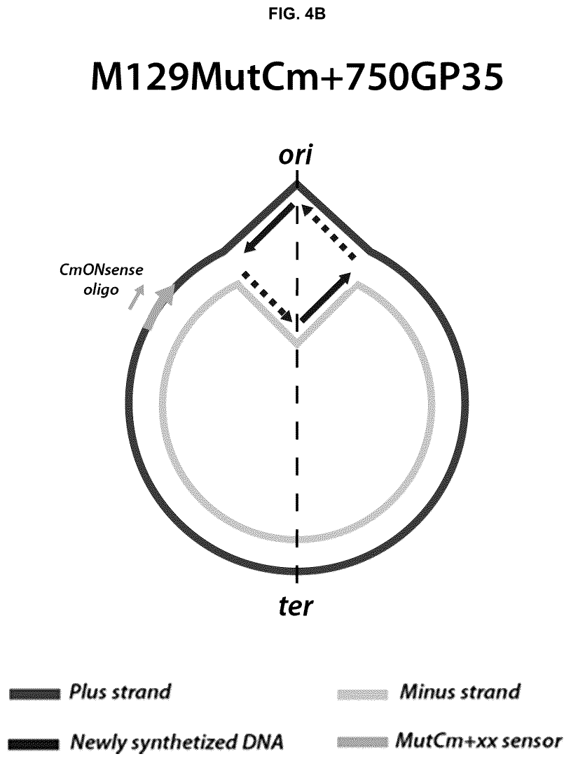

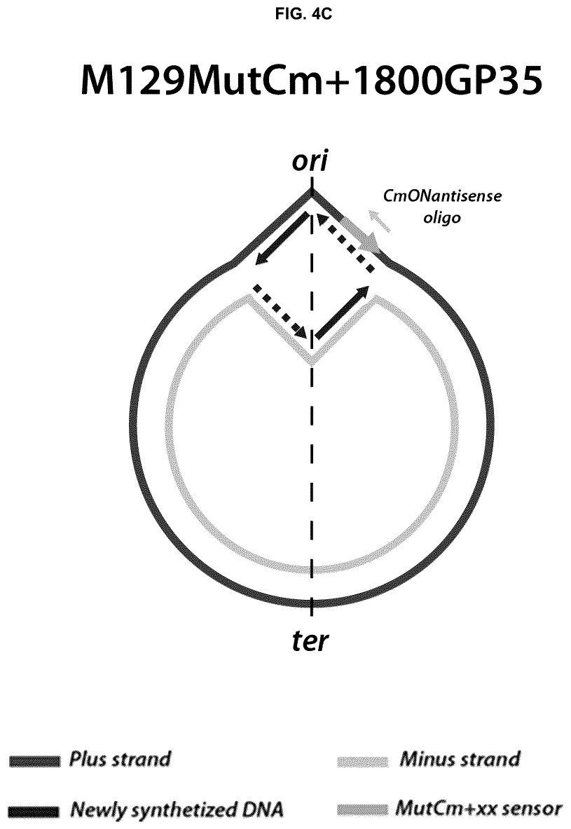

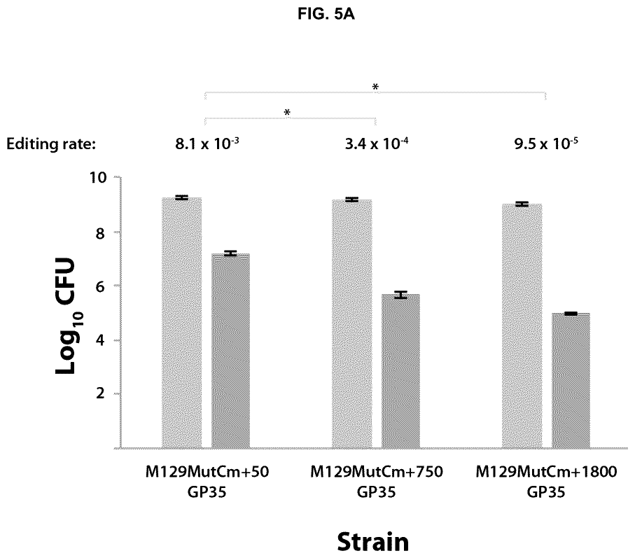

Apart from the interest that 1-bp gene editing could help to elucidate the role of genes with unknown functions, the future adoption of M pneumoniae as a suitable synthetic biology chassis strain would unarguably require modifications that go beyond the introduction of certain point mutations to its genome. Therefore, we determined which efficiencies could be obtained when using GP35-mediated oligo recombineering to carry out gene editions larger than 1 bp. To this end, and inspired by the MutCm+1 recombineering sensor, we created three new reporters in which a 50-bp, 750-bp, or 1800-bp frame-shifting sequence was placed in the cat coding sequence. The above-mentioned sensors were introduced into M. pneumoniae WT cells by means of transposon vector transformations. The A-PCR analysis conducted in individual clones obtained from these transformations revealed that the transposons carrying the different recombineering sensor were inserted in the positive strand of the MPN493, MPN582, and MPN034 loci, respectively. The resulting clonal strains carrying the different recombineering sensors were subsequently transformed with a second transposon vector coding for GP35 recombinase. Individual clones of these transformations were grown to generate three different clonal reporter strains termed M129MutCm+50GP35, M129MutCm+750GP35, and M129MutCm+1800GP35. Given the chromosomal locations of the sensors in these reporter strains (FIGS. 4A-4C), the CmONsense oligonucleotide was used as a recombineering substrate for both M129MutCm+50GP35 and M129MutCm+750GP35 strains, whereas the CmONantisense oligonucleotide was used for the M129MutCm+1800 strain. The three reporter strains were then subjected to 6 electroporation pulses with 5 μl of their corresponding oligos, and after 24 hours, serial dilutions of the transformations were seeded to obtain the amount of total and edited cells, as well as the editing rate achieved for each modification (FIG. 5A).

For all three reporter strains, there was a clear anti-correlation between recombineering efficiency and length of the attempted deletion (FIG. 5B). More specifically, a 50-bp deletion produced 1.5×10⁷ positively-edited cells, as inferred from their ability to grow on Cm-selective plates. Indeed, when this value was compared to the total amount of cells, the resulting editing rate obtained for the 50-bp deletion was 8.1×10⁻³, which is only 3-fold lower than that observed for a 1-bp deletion (2.7×10⁻²). In contrast, deletions of 750 bp and 1800 bp occurred at much lower frequencies, with editing rates of 3.4×10⁻⁴ and 9.5×10⁻⁵, respectively. Collectively, these results showed that GP35-mediated oligo recombineering can perform targeted chromosomal deletions of various sizes, although the efficiency is affected by the length of the deletion. Indeed, when the editing rates obtained for each reporter strain are plotted against the deletion size, it becomes evident that the results are characterized by a decreasing power trend (FIG. 5B). Of note, the different recombineering sensors were accommodated at distant chromosomal locations (i.e. MPN049, MPN493, MPN582, and MPN034), suggesting that the whole chromosome is susceptible of being edited by GP35-mediated oligo recombineering.

The sequences (gene and encoded protein) of GP35, Cre and vCre, Puromycine resistance, and the nucleotide sequence of the oligos used for editing in the examples are depicted in FIGS. 6A and 6B.

### 5. Generation of a Nuclease Counterselection System.

The main limitation of oligo recombineering resides in its inability to select for those cells carrying the intended modification, as the limited length of the oligonucleotides precludes the inclusion of a selection marker into the chromosome of edited cells in order to facilitate their identification. To overcome this limitation, spCas9, a Streptococcus pyogenes-derived protein, part of the widely known CRISPR/Cas system (Jiang et al., RNA-guided editing of bacterial genomes using CRISPR-Cas systems, Nature Biotechnology, 2013), has been recently repurposed as counterselection tool for recombineering protocols (Reisch et al., The no-SCAR (Scarless Cas9 Assisted Recombineering) system for genome editing in Escherichia coli, Scientific Reports, 2015) in view of its ability to specifically cleave a target DNA sequence in an easily reprogrammable manner. This ability relies on short guide RNAs (sgRNAs) that guide the endonuclease Cas9 to their complementary strand on the target DNA, and also on the presence of a 5′-NGG-3′ consensus sequence immediately downstream of the target site, which is called the protospacer adjacent motif (PAM). Cas9 chromosomal cleavage is highly lethal in bacteria, presumably due to the lack of NHEJ systems in most commonly studied bacteria (Cui et al., Consequences of Cas9 cleavage in the chromosome of Escherichia coli, Nucleic Acids Research, 2016). This toxicity has been exploited to counter select non-edited cells in oligo recombineering protocols developed for different strains (inter alia Penewit et al., Efficient and Scalable Precision Genome Editing in Staphylococcus aureus through Conditional Recombineering and CRISPR/Cas9-Mediated Counterselection, MBio, 2018, and Aparicio et al., CRISPR/Cas9-Based Counterselection Boosts Recombineering Efficiency in Pseudomonas putida, Biotechnology Journal, 2018).

For this reason, the transposon vector employed to introduce the GP35 recombinase into the three reporter strains also contained a Cas9-based counterselection platform. Specifically, this platform was composed of: (i) an inducible promoter responding to anhydrotetracycline (aTc), termed Pxyl/tetO2mod (Mariscal et al., All-in-one construct for genome engineering using Cre-lox technology, DNA Research, 2016), (ii) a copy of the enhanced-Cas9 (eCas9) coding sequence (Slaymaker et al., Rationally engineered Cas9 nucleases with improved specificity. Science, 2016), and (iii) a sgRNA termed eNT2 that targets the non-template strain of the gene coding for the Venus fluorescent protein (Qi et al., Repurposing CRISPR as an RNA-Guided Platform for Sequence-Specific Control of Gene Expression, Cell, 2013). Note that the sequence recognized by eNT2 sgRNA is present in the three different recombineering sensors, as part of the frame-shifting sequences. Thus, in principle, only edited cells —that is, those that have incorporated a recombineering oligo and consequently deleted the Cm frame-shifting sequence—can survive once eCas9 expression is induced. Nevertheless, non-edited “escapee” cells carrying mutations that somehow affect Cas9 activity or expression would also survive and still carry the sequence recognized by eNT2 sgRNA in their chromosomes (also termed Cas9 evaders). Remarkably, the proportion of evaders in a population largely influences the outcome of the recombineering protocol. Specifically, if the proportion of evaders is higher than the proportion of edited cells, the selection of the edited cells requires numerous clones to be screened. In contrast, if the rate of evaders is lower than the rate of editing, virtually all cells surviving Cas9 expression should carry the intended modification.

Thus, the three different reporter strains expressing GP35 recombinase and the eiCas9 system, were transformed with either CmONsense or CmONantisense oligos, and after 24 hours, seeded on Cm-selective or non-selective plates supplemented with different aTc concentrations. This allowed us to determine the inducer concentrations at which the expression levels of eiCas9 promoted a marked decrease in cell viability of non-edited cells, without compromising the survival of edited cells. In addition, data from the highest aTc concentrations enabled us to determine the eiCas9 evader rate for each strain.

The M129MutCm+50GP35 strain had an initial editing rate of 1.3×10⁻² (FIG. 7A), but had an evader rate of 3.1×10⁻³. This is inferred by dividing the number of survival cells at highest aTc concentration, 3.7×10⁶, by the total cell number, 1.2×10⁹. Thus, the ratio between the editing rate and the evader rate for this strain suggests that there should be an aTc concentration in which almost all survivors carry the intended modification. Indeed, eiCas9 expression induced at low levels (with 0.33 ng/ml aTc) led to a 30-fold reduction in viability of total cells (1.2×10⁹ vs. 3.8×10⁷) but did not affect the viability of the edited population (1.5×10⁷ vs. 1.3×10⁷). This boosted the editing rate from an initial value of 1.3×10² to 3.5×10⁻¹. Increasing the aTc concentration to 0.66 ng/ml further increased the editing rate, to 9.9×10⁻¹. Inducer concentrations above this value rendered editing rates comparable to the ones obtained without induction. This suggests that the protein levels obtained at these concentrations are high enough to lose eiCas9 sequence specificity, leading to a decrease in cell viability that equally affects edited and non-edited cells (i.e. off-target activity). The editing rate obtained at 0.66 ng/ml of aTc (of 9.9×10⁻¹) implies that virtually all cells that survive eiCas9 induction at this concentration are edited cells. To further confirm this, we randomly pick 20 colonies from non-selective plates supplemented with 0.66 ng/ml aTc and inoculate them into a 96-well plate containing either non-selective or Cm-selective medium (FIG. 7A). Notably, 19 of the 20 colonies were able to grow on the Cm-selective medium.

For the M129MutCm+750GP35 strain, we found that the initial editing rate after transforming CmONsense oligo was 4.9×10⁻⁴ (FIG. 7B), and that the eiCas9 evader rate for this strain was 3.9×10-. In other words, the frequency of edited cells was eight times lower than the frequency of cells that do not respond to eiCas9 induction, making selection of edited cells a challenge. Specifically, similar to what we observed for the M129MutCm+50 strain, we found a window of aTc concentrations at which the total cell viability of M129MutCm+750 strain was compromised without affecting the survival of edited cells. However, this window comprised aTc concentrations that are lower for M129MutCm+50 strain than for the M129MutCm+750 strain. Noteworthy, these two strains are clonal populations with different chromosome locations of the eiCas9 gene cassette. Thus, it seems that the gene context of the inserted cassette affects how much inducer concentration is required to obtain an effective eiCas9 counterselection. Regardless of this aspect, when the eiCas9 gene cassette was induced at 1.25 ng/ml aTc, the editing rate increased from the initial value of 4.8×10⁻⁴ to 1.2×10⁻¹. While this editing rate may be further improved in future work, the current protocol renders screening for edited clones a feasible endeavor for any present molecular biology lab. For instance, analysis of 20 clones randomly picked from the non-selective plate that had been supplemented with 1.25 ng/ml inducer revealed the presence of two clones (i.e. #6 and #9) that were resistant to Cm and thus had been edited (FIG. 7B).

Finally, for the M129MutCm+1800GP35 strain, the inducer concentrations required for effective eiCas9-mediated counterselection resembled those determined for the M129MutCm+750 strain (FIG. 7C). Remarkably, the evader rate for this strain was found to be 8.3×10⁻⁴ (Table S4), a value lower than that of the M129MutCm+50 and M129MutCm+750 strains (3.1×10— and 3.9×10⁹, respectively) or of that reported for P. putida (5.8×10⁻³). However, this value is still high when compared to the evader rate reported for E. coli (Reisch et al., The no-SCAR (Scarless Cas9 Assisted Recombineering system for genome editing in Escherichia coli, Scientific Reports, 2015) and Lactobacillus reuteri (2.5×10⁻⁴) (Oh et al., CRISPR-Cas9-assisted recombineering in Lactobacillus reuteri. Nucleic Acids Research, 2014). The reasons underlying these divergences in the evader rates even within the same species are not clear. However, we hypothesized that an increased evader rate might be related to the inability to tightly control the expression levels of Cas9, which would eventually lead to a higher proliferation of cells carrying mutations in the counterselection system. Indeed, it seems that the expression of the eiCas9 cassette is controlled more tightly in M129MutCm+1800GP35 than in the other reporter strains that carry this cassette in other chromosome locations. Regardless, the initial editing rate for this strain was 1.1 ×10⁻⁴, and induction of eiCas9 with 1.25 ng/ml of aTc enhanced this rate to 6.4×10⁻². This is in line with results of the screening of 20 randomly-picked clones from the non-selective plate supplemented with 1.25 ng ml aTc, in which one clone (#16) was found to be Cm resistant and thus edited (FIG. 7C).

Altogether, these results indicate that GP35 recombineering coupled to eiCas9 counterselection enables cells that have undergone a 50-bp chromosome edition to be isolated with virtually no screening, and allows simple and affordable screening experiments for deletions as large as 1.8 kb. Furthermore, a more tightly regulated inducible system might make it possible to decrease the evader rate to levels similar to the ones reported for E. coli or L. reuteri, thereby facilitating selection of modifications even larger than 1.8 kb. The sequence of alternative genes and proteins that can be used for counterselection are depicted in FIGS. 10A-10G (including different variants of Cas9, Barnase, restrictase Scel, DnaseI, and SssB).

### 6. Knock-In of a Site-Specific Recombinase Site in Mycoplasma Mediated by Recombineering.

On the first day of the protocol, 0.5 nmol of an editing oligonucleotide termed MOΔ50 was transformed into a Mycoplasma pneumoniae strain expressing GP35 from a constitutive promoter and Cre recombinase from the inducible Ptet promoter. A mock transformation without oligo was used as negative control.

The total volume of both transformations was inoculated into T75 flasks containing 25 ml of Hayflick medium supplemented with 5ng ml−1 of anhydrotetracycline and the resulting cultures were allowed to grow for 24 hours at 37° C. and 5% CO².

Next day, the tenth part of the cultures was transformed with pUC57PuroSelector plasmid. Two hours post-transformation the whole volume of both transformations was inoculated into T75 flasks containing 25 ml of Hayflick medium supplemented with 5 ng ml−1 of anhydrotetracycline and 3 μg ml−1 puromycin. Both flasks were grown for 24 hours at 37° C. and 5% CO₂.

The third day of the protocol cells were scraped out of the flasks in a total volume of 500 μl of Hayflick medium, and half of this volume was spread onto Hayflick 0.8% bacto agar plates supplemented with 3 μg ml-i puromycin. Plates were incubated at 37° C. and 5% CO² for a minimum period of 10 days before screen the resulting colonies.

GP35-mediated incorporation of MOΔ50 oligonucleotide into M pneumoniae genome leads to the replacement of 50 bp of MPN506 gene by the 34 bp lox71 site. In this way, the deletion of these 50 bp does not provide any selectable phenotype but generates a landing pad that could be subsequently loaded. In order to do that, pUC57PuroSelector plasmid is later transformed leading to the integration of the whole plasmid, and the associated Puromycin resistance marker, into the previously deleted locus. In this way, when the transformations are seed over Puromycin supplemented plates only those cells that have been edited would grow (FIGS. 8A and 8B). Noteworthy, it is important to always run in parallel a control that in the first day of the protocol did not receive the oligo, but received the plasmid. This control should reveal the rate of spurious plasmid-chromosome recombination, which would result in a Puromycin resistant phenotype without carrying an edited locus.

### Further Optimization

0.5 nmol of different editing oligos (FIGS. 6A and 6B) were co-transformed with pUC57PuroSelector plasmid into a Mycoplasma pneumoniae strain expressing GP35 from a constitutive promoter and Cre recombinase from the inducible Ptet promoter (FIGS. 9A-9D). Also, a mock transformation without oligo was performed as a control. Transformed cells were allowed to recover for at least 3 hours in Hayflick medium at 37° C.

In order to mediate the integration of pUC57PuroSelector plasmid that will allow the selection of edited clones, transient expression of Cre recombinase is required. To do that, the whole volume of the oligo+plasmid co-transformations was inoculated into T75 flasks containing 25 ml of Hayflick medium supplemented with 5 ng ml−1 of anhydrotetracycline and 3 μg ml−1 puromycin. These cultures were allow to grow in the presence of inducer for a period of time of minimum 12 hours and maximum 72 hours. After this incubation, cells were scrapped out of the flasks in a total volume of 500 μl of Hayflick medium, and half of this volume was spread onto Hayflick 0.8% bacto agar plates supplemented with 3 μg ml−1 puromycin. Plates were incubated at 37° C. and 5% CO2 for a minimum period of 10 days before screen the resulting colonies.

Alternatively, induction of Cre expression may be performed directly on plates. In such an approach, after the recovery time from the oligo+plasmid co-transformations half of the volume is spread onto Hayflick 0.8% bacto agar plates supplemented with 3 μg ml−1 puromycin and 1 ng ml−1 anhydrotetracycline. Plates were incubated at 37° C. and 5% CO2 for a minimum period of 10 days before screen the resulting colonies.

### 7. Generation of a Modified Mycoplasma Strain Using the Oligonucleotide Modification System.

In bacteria, most of the intracellular proteolysis is mediated by ATP-dependent proteases belonging to the AAA+family proteins (Bittner et al., mini review: ATP-dependent proteases in bacteria, Biopolymers, 2016), including FtsH. FtsH is capable to unfold and digest folded proteins due to their ATPase unfoldase activity (Sauer and Baker, AAA+proteases: ATP-fueled machines of protein destruction, Annual review of biochemistry, 2011). FtsH is essential for cell growth in M. pneumoniae, which has hampered the isolation and characterization of null mutants so far. To study the cellular function of this protease, we overcome these difficulties by generating for the first time conditional mutants in M pneumoniae. To this end, we used the genome-editing tools of the present disclosure which rely on the phage recombinase GP35 to control FtsH expression through a Tet inducible system. A schematic representation of the genome editing that was performed for obtaining the mutant strain is illustrated in FIG. 11A. Briefly, to achieve conditional FtsH expression, we first introduced the ftsH gene under the control of the inducible platform by transposon delivery, and subsequently replaced the endogenous fstH gene by a cat selectable marker (FIG. 11A).

Transformants were isolated using chloramphenicol agar plates and modification was confirmed by PCR screening (FIG. 11B). FtsH expression was monitored by RNA-seq and Western blot assays under inducing and depleting conditions, showing a good repression-induction transcriptional pattern, which correlated with protein expression (FIG. 11C). We also compared the growth rate after FtsH depletion (FIG. 11D). Consistent with their reported essentiality, depletion of this proteases inhibited growth.

### Bacteral Strains and Growth Conditions

Wild-type M pneumoniae strain M129 and its derivatives were grown in modified Hayflick medium at 37° C. under 5% CO₂ in tissue culture flasks, unless otherwise indicated. Depending on the specific condition, Hayflick medium was supplemented with 0.8% agar, puromycin (3 μg/ml), chloramphenicol (20 μg/ml) or tetracycline (2 μg/ml) for selection of transformants. E. coli strain TOP10 (Invitrogen) was used for vector cloning. This strain was grown at 37° C. in LB broth or LB agar plates containing ampicillin (100 μg/ml) and X-Gal (40 μg/ml) as needed.

### Construction and Mutant Design

FtsH (ΔIndFtsH) conditional mutant was constructed using genome-editing tools mediated by the phage recombinase GP35 according to the invention herein. The specific genome editions were performed as described below (see also FIG. 11A for illustration).

First, we introduced by transposon delivery the ftsH gene under the control of the inducible platform in the M129_GP35 strain (wild-type strain expressing the gp35 gene), hereby generating the M129+pMTnTc_IndFtsH strain. The transposon vector (pMTnTc_ftsH_Ind) used to generate this strain was obtained by cloning into a pMTnTetM438 vector, the ftsH inducible platform containing the tetR and the ftsH gene under the control of Pxyl/TetO2 promoter. Molecular cloning was performed by Gibson assembly as detailed in Table 1 using the primers listed in Table 2. Then, we deleted the endogenous ftsH gene in M129+pMTnTc_IndFtsH strain by transforming an ssDNA recombineering substrate containing the cat selectable marker enclosed by ftsH flanking regions. The cat selectable marker was flanked by lox sites, allowing cat excision by the Cre recombinase. In total, 92% of the endogenous ftsH coding sequence was deleted, leaving 50 bp and 100 bp at the 5′ and 3′ ends, respectively, to preserve overlapping regions with flanking genes. To prevent transcriptional polar effects on the downstream genes we also included a promoter after the cat gene that replaced the ftsH endogenous locus.

TABLE 1
Gibson cloning strategy for plasmids used
to obtain the FtsH conditional mutant.

| | | | |
|---|---|---|---|
| Gibson | Primers | Template | Gibson |

pMTnTc_ftsH_Ind

| | | | |
|---|---|---|---|
| PCR1 | ter625_TetR_F/ftsH_IndPr_R | pΔLonPr_Ind ⁽¹⁾ | |
| PCR2 | p_ter625_F/ftsH_IndPr_R | PCR1 | x |
| PCR3 | IndPr_LAFtsH_R/p_ftsH_R | gDNA M129 | x |
| Vector | | pMTnTetM438 | x |
| | | EcoRV | |

pΔFtsH

| | | | |
|---|---|---|---|
| PCR1 | p_LA_ftsH_F/ | gDNA M129 | x |
| PCR2 | P438_lox66_F/ | pMTnCat(lox) ⁽³⁾ | x |
| PCR3 | lox71_RA_ftsH_F/ | gDNA M129 | x |
| Vector | | pBSK EcoRV | x |
| | | digested | |

⁽¹⁾ In-house cloning vector containing the tet inducible platform.
(2) Pich et al., Comparative analysis of antibiotic resistance gene markers in Mycoplasma genitalium: application to studies of the minimal gene complement, Microbiology, 2006.
⁽³⁾ In-house cloning vector containing the cat gene flanked by lox sites.

TABLE 2
Primers used in example 7.

| | |
|---|---|
| Primer name | Sequence (5′ to 3′)^{a} |

Construction of plasmids

| | |
|---|---|
| pMTnTc_ftsH_Ind | |
| p_ter625_F | ACGGTATCGATAAGCTTGATAAAAAATACCTG |
| | AGTCTTTCAGGTATT (SEQ ID NO: 31) |
| ter625_TetR_F | TACCTGAGTCTTTCAGGTATTTTTTTTTAAGA |
| | CCCACTTTCACATTTA (SEQ ID NO: 32) |
| ftsH_IndPr_R | TTTTTTTCATATGCTCTATCAATGATAGAGG |
| | (SEQ ID NO: 33) |
| IndPr_LAFtsH_R | GATAGAGCATATGAAAAAAAATAAAGGACTTA |
| | ACG (SEQ ID NO: 34) |
| p_ftsH_R | TCTAAATACTAGAATTCGATTTAACTGTTTGT |
| | TTCACTGTCT (SEQ ID NO: 35) |
| pΔFtsH | |
| p_LA_ftsH_F | ACGGTATCGATAAGCTTGATCTTGTACTGCCT |
| | TTTGGTGC (SEQ ID NO: 36) |
| P438_LA_ftsH_R | GTATTTAGAATTAATAAAGTGCAAGAACAGCA |
| | AGCCAAAC (SEQ ID NO: 37) |
| P438_lox66_F | ACTTTATTAATTCTAAATACTATACCGTTCGT |
| | ATAGCATAC (SEQ ID NO: 38) |
| RA_ftsH_lox71_R | ACTACGAGCGAAAAACCGCATACCGTTCGTAT |
| | AATGTATG (SEQ ID NO: 39) |
| lox71_RA_ftsH_F | TGCGGTTTTTCGCTCGTAGT (SEQ ID NO: |
| | 40) |
| p_RA_ftsH_R | CCGGGCTGCAGGAATTCGATCCTAAAGGCAAG |
| | GACATTCTT (SEQ ID NO: 41) |

Production of ssDNA recombineering
substrates and PCR screening

| | |
|---|---|
| Pro_KOftsH_F | C*T*T*G*TACTGCCTTTTGGTGC (SEQ ID |
| | NO: 42) |
| Bio_KOftsH_R | [biotin]CCTAAAGGCAAGGACATTCTT |
| | (SEQ ID NO: 43) |

^{a}underlined sequences indicate the priming sequences. Depending on the primer, the remaining sequence of the primer was designed to introduce promoter and terminator sequences or overlapping sequences for Gibson assembly.

Production of ssDNA Recombineering Substrates

The recombineering substrates to perform the genome modifications described above were obtained as follows. To delete the endogenous ftsH gene, we cloned the cat selectable marker enclosed by ftsH flanking regions into a pBSKII+by Gibson cloning generating plasmid pAftsH (Tables 1 and 2). PCR templates to generate ssDNA recombineering substrates were obtained using pAftsH plasmids as templates and the pair of primers Pro_KOftsH_F/Bio_KOftsH_R, respectively (Table 2). These primers contained biotin or phosphorothioate modifications attached to the 5′ ends, allowing both ssDNA purification and protection of the ssDNA substrate. To generate ssDNA substrates, 120 l of Streptavidin dynabeads (MyOne™ Streptavidin Cl, Invitrogen) were washed three times with washing buffer (10 mM Tris, 1 mM EDTA, 2M NaCl, pH7.5), and incubated with 20 μg of the corresponding PCR product by rotation at RT for 2h. Dynabeads were then recovered and resuspended in 50 l of melting buffer (125 mM NaOH). After a gently vortex mixing, magnetic beads were pulled down and the supernatant solution recovered and diluted in 500 μl of neutralization buffer (60 mM NaAc in TE buffer). A second round of elution was performed and recovered to the same neutralization solution. The ssDNA was precipitated by adding 60 μg of glycogen and 1 volume of isopropanol. After 30 min of incubation at RT, ssDNA was recovered by centrifugation (14000 rpm, 45 min at 4° C.), and the pellet washed twice with chilled 70% ethanol. Finally, the pellet was air dried and resuspended in electroporation buffer (8 mM HEPES, 272 mM sucrose, pH 7.4).

### Transformation and Isolation of Mutants

To obtain FtsH conditional mutants, M129_GP35 and M129+pMTnTc_IndFtsH strains were transformed respectively with 3 μg of the corresponding ssDNA recombineering substrate (see above). Bacteria transformation was accomplished by electroporation. To allow GP35 mediated recombination, electroporated cells were cultured in 25 cm² flasks containing 5 ml of Hayflick medium during 24h. Then, cells were recovered and mutants selected in Hayflick agar plates containing 20 μg/ml chloramphenicol and 100 ng/ml tetracycline to induce FtsH expression. The intended genetic modifications were confirmed by PCR screening as shown in FIG. 11B. Genetic edits were further confirmed by RNA-seq mapping. The specific transposon insertion sites in each of the strains were also determined by RNA-seq mapping. In particular, the transposon expressing the gp35 gene was located in coordinate 613384 in the ΔIndFtsH mutant. The pMTnTc_ftsH_Ind mintransposon in the ΔIndFtsH mutant was located in the coordinate 372403.

### Culture Conditions for FtsH Depletion

M pneumoniae strain ΔIndFtsH was grown in 5 ml cultures supplemented with 20 μg/ml of chloramphenicol and 10 ng/ml of tetracycline to induce FtsH expression. After 48h of culture, cells were washed with Hayflick twice and scraped off from the flasks in 5 ml of fresh medium without tetracycline. To grow cells under inducing or depleting conditions, new 5 ml cultures containing plain medium (depletion) or supplemented with 100 ng/ml tetracycline (induction) were inoculated with 1:5 or 1:12.5 dilutions of cell suspension, respectively. Protein or RNA samples were obtained after 72h of depletion.

### Growth Curve Analyses

Preparation of starting inocula and growth curve analyses (based on the “growth index” method) were performed as previously described (Yus et al., Determination of the Gene Regulatory Network of a Genome-Reduced Bacterium Highlights Alternative Regulation Independent of Transcription Factors, Cell Systems, 2019). Here, the conditional mutants were grown in inducing conditions to exponential phase and washed twice in Hayflick medium before inoculation in the presence or absence of tetracycline (100 ng/ml).

### RNA Sample Preparation and RNA-Seg Analyses

M pneumoniae conditional mutants were grown in inducing or depleting conditions as described above per duplicate. Before RNA isolation, the culture medium was changed with fresh one and the cells further incubated for 6h. At this point, cells were washed with PBSx1 and lysed immediately with 70001 Qiazol (Qiagen). RNA isolation was performed using the miRNeasy kit (Qiagen) following the manufacturer's instructions, including the in-column DNase I treatment. The quality of RNA (amount and integrity) was assessed using a BioAnalyzer (Agilent). RNA-seq libraries were prepared at the CRG ultrasequencing facility using the TruSeq Stranded mRNA Sample Prep Kit v2 according to the manufacturer's protocol using the following modifications. The poly(A) selection step was omitted and fragmentation was done using 100 ng total RNA as starting material. To maintain smaller library insert sizes than in the standard protocol, the first AMPure XP purification after adaptor ligation was performed using 50 ul AMPure XP beads instead of 42 ul. The second round of bead purification was then performed using 55 ul AMPure XP beads instead of 50 ul. The purification of the PCR reaction after library amplification was done using 55 ul AMPure XP beads instead of 50 ul. Sequencing was performed using a HiSeq 2500 (Illumina) with HiSeq v4 chemistry and 2×50 bp paired-end reads.

Processing of sequencing reads was performed as follows. Adapter sequences were trimmed from short paired-end reads by using the SeqPurge tool (version 0.1-478-g3c86511b) (Sturm et al., SeqPurge: highly-sensitive adapter trimming for paired-end NGS data, BMC bioinformatics, 2016). keeping trimmed reads with a minimum length of 12. Reads were aligned to the wild-type genome of M pneumoniae a129 (NCBI accession NC 000912.1) and to the transposon inserts sequences using bowtie2 v. 2.3.5 (Langmead and Salzberg, Fast gapped-read alignment with Bowtie 2, Nature Methods, 2012), with parameters values: end-to-end mode, 0 mismatches (−N). seed length of 20 n (−L), very sensitive mode (−L 20 -D 20 -R 3 -i 'S,1,0.50′), maximum fragment length 1200 ti (−X), only best alignment reported (−k 0). Alignment files were converted from SAM format to sorted indexed BAM format using samtools v. 1.9 (using htslib 1,9) and sort (GNU coreutils) 8.26. Reads were further filtered by a minimum quality (MAPQ) threshold of 15, keeping only primary and mapped reads, and converted to sorted BEDPE format using sartools and bedtools v2.27.1. Fragments counts per annotation region were computed using bedtools, with strand specific overlaps with minimum overlap fraction of 0.5 of read length. Finally, strand-specific per-base coverage was computed using bedtools.

### SDS-PAGE and Immunoblot Analyses

Mycoplasma cell lysates were quantified using the Pierce™ BCA Protein Assay Kit and 10 μg of cell extracts were subjected to electrophoresis through NuPAGE™ 4-12% Bis-Tris pre-cast polyacrylamide gels (Invitrogen). Proteins were then transferred onto nitrocellulose membranes using an iBlot™ dry blotting system (Invitrogen). For immunodetection, membranes were blocked with 5% skim milk (Sigma) in PBS containing 0.1% Tween 20 solution and probed with polyclonal antibodies specific to mycoplasma FtsH (1:3,000) (kind gift of Dr. Herrmann, Heidelberg University). As a loading control, we used a polyclonal anti-CAT (abeam) antibody (1:2,000). Anti-rabbit IgG (1:5000) conjugated to horseradish peroxidase (Sigma) were used as a secondary antibody. Blots were developed with the Supersignal™ West Pico Chemiluminescent Substrate detection Kit (Thermo Scientific) and signals detected in a LAS-3000 Imaging System (Fujifilm).

### 8. Assessing Toxicity of a Modified Mycoplasma Strain.

Preparation of Mycoplasma cultures and infection of animals Mycoplasma strains are grown in a T75 cm² flask with 25 ml of Hayflick medium and 50ul of cells from the stock. After three days of growth the color of the medium changes from red to orange because the acidification by Mycoplasma growth. At this point the flask is confluent and the expected biomass is 10 CFUs in the T75 flask.

Mycoplasma cells grow attached. First, the medium is removed from the flask. Then, we add 50ul of PBS and scrap the cells to detach them from the surface of the flask. We increase the volume to 1 ml of final volume so the expected concentration of the cells is approximately 10\hat{ }10 CFUs/ml. After passing through an insulin syringe (to disaggregate cells), serial dilutions of these inoculum should be done and 10 μl of dil 10-6, 10-7, 10-8 should be plated to count the CFUs to be sure about the titer used in the specific study. The colonies can be counted after 1 or 2 weeks so to identify the inoculum that is used in the experiment.

Mice were inoculated with different doses (10\hat{ }3, 10\hat{ }5 and 10\hat{ }7 CFUs) of Mycoplasma strains wild type and chassis (WT and CV2) were injected into the mammary glands of the animals as shown in FIG. 12A.

After 4 days of infection maintenance of Mycoplasma in the tissue, macroscopic lesions and the immune response is measured (FIG. 12B).

### Maintenance of Mycoplasma in the Mammary Gland Tissue

Animals were sacrificed after 4 days of infection, mammary gland was extracted and the tissue was weight to estimate the mass. After homogenizing the tissue, eukaryotic cells and debris were eliminated by centrifugation. Then, the supernatant was recovered and passed through a syringe (to disaggregate Mycoplasma cells). Samples were spread on agar plates with Hayflick medium and colony forming units (CFUs) were counted using a Zeiss STEMI 2000-CS lupe. The values of Mycoplasma recovered after four days of infections are shown in FIGS. 13A and 13B.

### Macroscopic Lesions in Mammary Gland Tissue

Study of macroscopic lesions caused by the strains in the mammary gland tissue revealed hemorrhagic lesions caused by WT strain. These lesions were not found in the animals infected with the chassis (CV2) (FIG. 14).

### Characterization of Immune Response in the Infection Caused by Mycoplasma Strains

Immune response in mammary gland of the mice caused by WT and Chassis strains was measured after mRNA extraction of the tissue and quantification of different cytokines by qRT-PCR (Table 3). TNF-A, IL1b and IL6 showed significant differences between WT and Chassis strains (FIG. 15), being the levels of those interleukins lower in the Chassis strain. These results reveal that inflammatory response caused by WT strain is reduced when virulence factors have been depleted.

TABLE 3
Quantification of cytokines by qRT-PCR.

| | CONTROL | WT | CV2 |
|---|---|---|---|
| TNF-A | 0.064 ± 0.042 | 1.530 ± 0.6* | 0.49 ± 0.221$ |
| KC | 0.786 ± 0.514 | 3.889 ± 0.915* | 2.895 ± 1.058* |
| INF-Y | 0.329 ± 0.296 | 0.633 ± 0.142* | 0.462 ± 0.288 |
| IL-1B | 0.024 ± 0.029 | 0.454 ± 0.254* | 0.172 ± 0.064 |
| IL-4 | 0.002 ± 0.002 | 0.003 ± 0.001 | 0.002 ± 0.002 |
| IL-6 | 0.035 ± 0.033 | 0.740 ± 0.558* | 0.295 ± 9.367 |
| IL-18 | 0.726 ± 0.497 | 1.632 ± 0.516 | 1.239 ± 1.076 |

*p < 0.05 Strains vs CONTROL
$p < 0.05 WT VS CV2 at 4 dpi

9. Transformation of M. pneumoniae with ssDNA-GP35 Complex

Here, experiments are described on transformation ofM pneumoniae with ssDNA-GP35 complex. After doing recombinant expression and purification of GP35 protein we formed GP35-ssDNA complexes in vitro. Then, we transformed the MutCm+1 sensor strain to evaluate the efficiency of the new approach and to compare with the previous approach were the GP35 protein is expressed constitutively from a gene inserted in the genome by transposition in the GP35MutCm+1 strain. We show that the transformation with GP35-ssDNA complex works by recovering cells resistant to chloramphenicol. Furthermore, the cat gene of these cells was sequenced and this sequencing showed that the mutation was corrected by recombination. It is the first time that transformation with a protein is reported for M pneumoniae.

Recombinant Expression and Purification of GP35 in E. coli.

The ORF of the GP35 protein was cloned in the pETM14 vector. pETM14-GP35 plasmid was used to express the GP35, in E. coli BL21 DE3 strain. Overnight culture has been diluted 1:100. Then 1 L culture has been grown until OD 0.6 at 37° C. Then, the culture was put in the incubator at 18° C. and induced with 0.2 mM IPTG for 16 hrs. The culture was centrifuged at 4000 rpm and 4° C. and the pellet was resuspended with lysis buffer: 50 mM Tris pH 8, 300 mM NaCl and 2 mM DTT. Then, cells were lysed by using the French press homogenizer. After centrifugation at 14.000 rpm to recover the soluble fraction, the supernatant was loaded to Hitrap 1 ml column and after washing steps protein was eluted with lysate buffer including 250 mM imidazole. The eluted protein was treated with precision protease for ON at 4° C. After treatment, the sample was reloaded in the column to remove the 6xHis tag. The protein was recovered from the flow trough of the purification and then concentrated by using the Vivaspin 10 column. 50 mM TRIS pH7.4, 300 mM NaCl, 1 mM DTT and 10% glycerol was used as recovery buffer. The protein was obtained at a concentration of 2.5 mg/ml and stored at −80° C.

### Formation of GP35-CmONsense Complex

Purified GP35 was mixed with CmONsense ssDNA. After lhl5 min of incubation at 37° C., the samples were brought to a final volume of 20 uL with electroporation buffer (8 mM HEPES, 272 mM sucrose, pH 7.4) and used immediately to transform M129MutCm+1 strains.

Transformation ofM pneumoniae.

GP35MutCm+1 and MutCm+1 strains were transformed with CmONsense and CmONsense-GP35 complex, respectively, by electroporation. Briefly, a frozen stocks of GP35MutCm+1 and MutCm+1 were diluted 1:100 with modified Hayflick broth supplemented with 2 μg/mL tetracycline and 3.3 μg mL-1 puromycin, and 2 μg mL-1 tetracycline, respectively. The cells were grown in 75 cm² tissue culture flasks containing 30 mL medium and incubated at 37° C. under 5% CO2 to late exponential growth phase (around 72 h of growth). Cells were washed twice, resuspended in precooled electroporation buffer (8 mM HEPES, 272 mM sucrose, pH 7.4), scraped off and passed through a 25-gauge (G25) syringe needle ten times. Aliquots of 50 μL of cells were placed in precooled 0.1 cm cuvettes and allowed to sit and kept on ice for 15 min. 20 μL of CmONsense and CmONsense-GP35 complex was added to the cuvettes containing M129MutCm+1GP35 and M129MutCm+1 cells, respectively, and were kept on ice for another 15 min. The cell mixes were electroporated by using a Bio-Rad gene pulser (1250 V, 25 uF, 100Ω). After 15 min on ice, 1 mL Hayflick broth was added and the cells were collected in a 2 mL microcentrifuge tube and incubated for 24 h at 37° C. in the presence of 5% CO2. 10 μL of M. pneumoniae transformed cells was spread on Hayflick agar petri dishes.

### Checking Single Clones to Check for the Recombination of CmR Gene

Colonies were picked up from the Hayflick agar petri dishes supplemented with 20 μg ml−1 chloramphenicol and were grown in a 25 cm2 tissue culture flasks containing 10 mL Hayflick broth medium supplemented with 20 μg ml−1 chloramphenicol and incubated at 37° C. under 5% CO2 for 7 days. Genomic DNA was extracted with the StrataClean resine (Cat. #400714) and a PCR was performed to amplify the cat gene and followed by Sanger sequencing.

10. Comparison of Recombineering Efficiencies in M. pneumoniae Between GP35-Oligo Complex Transformation Methods and GP35-Expressing M. pneumoniae Strains.

In a first step, the GP35 protein and ssDNA oligonucleotide were incubated in different ratios (complexes 1 to 3) as shown in Table 4. GP35 was produced and purified according to the procedure described in Example 9.

TABLE 4
Composition of complexes 1 to 3. GP35 stock solution buffer is
50 mM Tris pH 7.4, 300 mM NaCl, 1 mM DTT, and 10% glycerol.

| | Complex 1 | | Complex 2 | | Complex 3 | |
|---|---|---|---|---|---|---|
| GP35 protein (2500 ng/μl) | 0 | μl | 0 | μl | 3 | μl |
| CmONsense oligo (100 μM) | 0 | μl | 5 | μl | 5 | μl |
| Electroporation Buffer | 10 | μl | 5 | μl | 2 | μl |
| Final Volume | 10 | μl | 10 | μl | 10 | μl |

| | | | |
|---|---|---|---|
| Molar ratio GP35::ssDNA | 0 | 0 | 1:2 |

The compositions of complexes 1 to 3 were incubated at 37° C. for 75 minutes before transformation on strain c51 (M129 MutCm+1). After the electroporation pulse cells were recovered for 24 hours on Hayflick before seeding serial dilutions on Hayflick plates (to calculate the total number of cells) and Chloramphenicol plates (to calculate the number of edited cells).

In parallel, we made a control with strain C56 (M129GP35MutCm+1). As this strain expresses GP35, it was transformed only with the CmONsense oligo. This control serves to compare the efficiency of GP35 to catalyse oligo incorporation, when the protein is expressed by the strain or when is added exogenously. The results are depicted in Table 5.

TABLE 5
Editing efficiencies for the complexes depicted in Table 6, and
editing efficiencies for the C56 strain which expresses GP35.

| | | | | C56 |
|---|---|---|---|---|
| | C51 | C51 | C51 | strain + |
| | strain + | strain + | strain + | CmONsense |
| | Complex1 | Complex 2 | Complex 3 | oligo |
| Hayflick counts | 3 (−5) | 5 (−5) | 20 (−6) | 12 (−5) |
| Total number of | 1.5 × 10⁷ | 2.5 × 10⁷ | 1 × 10⁹ | 6 × 10⁷ |
| cells | | | | |
| Chloramphenicol | None | None | 5 (−3) | 6 (−4) |
| counts | | | | |
| Total number of | None | None | 2.5 × 10⁵ | 3 × 10⁶ |
| edited cells | | | | |
| Editing efficiency | None | None | 2.5 × 10⁻⁴ | 5 × 10⁻² |

From both Example 9 and Example 10 it can be concluded that the GP35 protein can catalyse oligo recombineering not only when is expressed by the cells subjected to the editing process, but also when the cells are transformed with a pre-assembled complex consisting of GP35 purified protein and an editing oligonucleotide. Noteworthy, the editing efficiency is lower when the recombinase is added exogenously, than when is expressed by the cells to be edited. However, is it likely that increasing the molar ratio GP35:ssDNA would lead to an increase of the editing efficiency, potentially reaching an editing efficiency similar to the one observed when the cells express GP35.

11. RNA-Based Delivery of GP35 into Mycoplasma.

GP35 RNA is transcribed from a GP35 DNA nucleotide sequence by a in vitro transcription experiment (In vitro transcription kit, Thermo Fisher) by following the instructions of the manufacturer. The complete protocol is conducted under RNAse free conditions, wherein both workstations and lab equipment is periodically cleansed with RNAseZAP cleaning agent (Sigma-Aldrich). Nuclease-free tubes (Eppendorf) are used in each step of the protocol. Ultrapure DEPC-treated water (Thermo Scientific) is used throughout the procedure up to the moment of electroporation.

Different sample solutions are prepared, reflecting complexes 1 to 3 from Example 9 wherein recombinant GP35 is exchanged for the GP35 RNA oligonucleotide (using stock concentrations of between 100 nM and 100 μM, depending on the yields of the RNA preparation. Complex volumes were adjusted appropriately and electroporation is performed as described in detail in Example 9. In accordance to Example 10, the C56 strain is used as a suitable positive control.

It can be concluded that RNA-based delivery of GP35 and a ssDNA recombineering template (e.g. CmONsense oligo) is a suitable alternative for achieving recombineering in M pneumoniae at efficiencies which may be further increased by extensive optimization.

### 12. Funding Acknowledgment

The projects leading to this application have received funding from the European Union's Horizon 2020 research and innovation programme and from the European Research Council (ERC) under grant agreements No 634942 (MycoSynVac) and No 670216 (MycoChassis), respectively.

1. An oligonucleotide modification system comprising:
1) a first element comprising at least one of:
(i) a nucleic acid comprising promoter or a functional variant or fragment thereof which is active in Mycoplasma bacteria, operably linked to a DNA nucleotide encoding a GP35 recombinase,
(ii) an RNA encoding a GP35 recombinase, and
(iii) a GP35 recombinase protein,
And
2) a second element comprising a nucleic acid, wherein the nucleic acid comprises two non-adjacent nucleotide sequences, each of the two non-adjacent nucleotide sequences having a minimum length of 5 nucleotides and wherein each of the two non-adjacent nucleotide sequences is a naturally occurring Mycoplasma sequence.

2. The oligonucleotide modification system of claim 1, wherein the two non-adjacent nucleotide sequences are separated from each other by a nucleotide sequence not-naturally occurring in Mycoplasma.

3. The oligonucleotide modification system according to claim 2, wherein the nucleotide sequence not-naturally occurring in Mycoplasma comprises a restriction site, a site-specific recombinase target site or a nucleotide-encoded selection marker or any combination thereof.

4. The oligonucleotide modification system of claim 1, wherein the GP35 recombinase is a GP35 recombinase having an amino acid sequence which is at least at least 85% identical, to the full length of SEQ ID NO: 1.

5. The oligonucleotide modification system of claim 1, further comprising a nucleic acid heterologous to Mycoplasma comprising a promoter or a functional variant or fragment thereof which is active in Mycoplasma bacteria, operably linked to a gene encoding a product.

6. The oligonucleotide modification system of claim 5, wherein the product comprises an exposure signal sequence or secretion signal sequence.

7. The oligonucleotide modification system of claim 1, further comprising a nucleic acid comprising a promoter or functional variant or fragment thereof which is active in Mycoplasma bacteria, operably linked to a sequence encoding a nuclease or a recombinase.

8. The oligonucleotide modification system of claim 7, wherein the nuclease is an endonuclease, a restriction enzyme, a meganuclease a zinc-finger nuclease (ZFNs), a transcription-activator like effector nuclease (TALENs), or a CRISPR associated (Cas)-based nuclease.

9. The oligonucleotide modification system claim 8, wherein the nuclease is a Cas9 nuclease which is at least 85% identical to the full length of SEQ ID NO: 2.

10. The oligonucleotide modification system of claim 9, further comprising
a nucleic acid capable of base pairing with a naturally occurring sequence in a Mycoplasma genome selected from
at least one single guide RNA sequence, or
at least one crRNA sequence and a tracrRNA sequence.

11. The oligonucleotide modification system of claim 1, wherein the system comprises a nucleic acid having at least 85% identity to the full length of SEQ ID NO: 3.

12. The oligonucleotide modification system of claim 1, wherein the system comprises a regulatory sequence capable of modulating transcription.

13. (canceled)

14. (canceled)

15. A method of altering the genome of a Mycoplasma bacterium, the method comprising:
introducing the oligonucleotide modification system of claim 1 or a GP35 recombinase protein, into a Mycoplasma bacterium.

16. The method according to claim 15, wherein the recombinant GP35 recombinase has an amino acid sequence that is at least to the full length of SEQ ID NO: 1.

17. (canceled)

18. The oligonucleotide modification system of claim 1, wherein the system is comprised in a Mycoplasma bacterium.

19. The oligonucleotide modification system of claim 3, wherein the nucleotide sequence not-naturally occurring in Mycoplasma is a lox site

20. The oligonucleotide modification system of claim 5, wherein the product is a therapeutic protein or an immunogenic protein.

21. The oligonucleotide modification system of claim 12, wherein the regulatory sequence is a riboswitch.


SEQUENCE LISTING (54 sequences)

SEQ ID NO: 1 - protein, 287 aa, Bacteriophage SPP1
MATKKQEELKNALAQQNGAVPQTPVKPQDKVKGYLERMMPAIKDVLPKHLDADRLSRIAMNVIRTNPKLLECDTASLMGAVLESAKLGVEPGLLGQAYILPYTNYKKKTVEAQFILGYKGLLDLVRRSGHVSTISAQTVYKNDTFEYEYGLDDKLVHRPAPFGTDRGEPVGYYAVAKMKDGGYNFLVMSKQDVEKHRDAFSKSKNREGVVYGPWADHFDAMAKKTVLRQLINYLPISVEQLSGVAADERTGSELHNQFADDDNIINVDINTGEIIDHQEKLGGETNE

SEQ ID NO: 2 - protein, 1370 aa, Streptococcus pyogenes
MNKPYSIGLDIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATLQEEKDYHEKFSTIYHLRKELADKKEKADLRLIYIALAHIIKFRGHFLIEDDSFDVRNTDISKQYQDFLEIFNTTFENNDLLSQNVDVEAILTDKISKSAKKDRILAQYPNQKSTGIFAEFLKLIVGNQADFKKYFNLEDKTPLQFAKDSYDEDLENLLGQIGDEFADLFSAAKKLYDSVLLSGILTVIDLSTKAPLSASMIQRYDEHREDLKQLKQFVKASLPEKYQEIFADSSKDGYAGYIEGKTNQEAFYKYLSKLLTKQEDSENFLEKIKNEDFLRKQRTFDNGSIPHQVHLTELKAIIRRQSEYYPFLKENQDRIEKILTFRIPYYIGPLAREKSDFAWMTRKTDDSIRPWNFEDLVDKEKSAEAFIHRMTNNDFYLPEEKVLPKHSLIYEKFTVYNELTKVRYKNEQGETYFFDSNIKQEIFDGVFKEHRKVSKKKLLDFLAKEYEEFRIVDVIGLDKENKAFNASLGTYHDLEKILDKDFLDNPDNESILEDIVQTLTLFEDREMIKKRLENYKDLFTESQLKKLYRRHYTGWGRLSAKLINGIRDKESQKTILDYLIDDGRSNRNFMQLINDDGLSFKSIISKAQAGSHSDNLKEVVGELAGSPAIKKGILQSLKIVDELVKVMGYEPEQIVVEMARENQTTNQGRRNSRQRYKLLDDGVKNLASDLNGNILKEYPTDNQALQNERLFLYYLQNGRDMYTGEALDIDNLSQYDIDHIIPQAFIKDDSIDNRVLVSSAKNRGKSDDVPSLEIVKDCKVFWKKLLDAKLMSQRKYDNLTKAERGGLTSDDKARFIQRQLVETRQITKHVARILDERFNNELDSKGRRIRKVKIVTLKSNLVSNFRKEFGFYKIREVNNYHHAHDAYLNAVVAKAILTKYPQLEPEFVYGDYPKYNSYKTRKSATEKLFFYSNIMNFFKTKVTLADGTVVVKDDIEVNNDTGEIVWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRADKLIILPKYSLFELENGRRRLLASAGELQKGNELALPTQFMKFLYLASRYNESKGKPEEIEKKQEFVNQHVSYFDDILQLINDFSKRVILADANLEKINKLYQDNKENISVDELANNIINLFTFTSLGAPAAFKFFDKIVDRKRYTSTKEVLNSTLIHQSITGLYETRIDLGKLGED

SEQ ID NO: 3 - DNA, 22 bp, Mycoplasma genitalium
tagtatttagaattaataaagt

SEQ ID NO: 4 - DNA, 864 bp, Bacteriophage SPP1
atggcaactaaaaaacaagaggaattaaaaaacgcactagcacaacaaaacggagccgttccgcaaacaccagtcaagccgcaggataaagtaaagggctatctcgaaagaatgatgccagctattaaagatgtgttgcctaagcatttagacgctgatcgactttcccgcatcgcaatgaacgtcattaggactaacccgaaacttctggaatgtgatacggcatcactcatgggggctgtgctggagtcagctaaattaggagtggagccgggacttttgggacaagcgtatattttgccgtacacgaattacaaaaagaaaacggttgaagctcagttcattcttggttataagggcctccttgatctcgtgagacgctctggacacgtgagcacaatcagcgctcaaactgtttacaaaaacgacacattcgaatatgaatatgggttagatgacaagcttgttcacagacccgcaccattcggaacagacagaggggagccagttggatactacgctgtagcgaaaatgaaggatggcggatataacttccttgtcatgagtaaacaggatgtcgagaaacaccgggacgccttttccaagtctaaaaacagagagggagtagtgtatggcccatgggctgaccacttcgacgcaatggcgaaaaagacagtcttgcgccagcttatcaactatttacctatcagtgttgagcaattatcaggagtagcggcagacgaacggacaggatcagaacttcataaccaatttgcggatgacgacaacattattaacgtcgatatcaatacaggtgaaatcattgaccatcaagaaaaattaggaggggaaacaaatgaatag

SEQ ID NO: 5 - DNA, 1032 bp, Bacteriophage P1
atgtccaatttactgaccgtacaccaaaatttgcctgcattaccggtcgatgcaacgagtgatgaggttcgcaagaacctgatggacatgttcagggatcgccaggcgttttctgagcatacctgaaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatgaatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaccctggatagtgaaacaggggcaatggtgcgcctgctggaagatggcgattag

SEQ ID NO: 6 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRTLDSETGAMVRLLEDGD

SEQ ID NO: 7 - DNA, 1143 bp, Vibrio parahaemolyticus
atgatagaaaaccaactctccctgctcggtgacttttccggagttaggccggatgatgtcaaaactgcgatccaagcggcccagaaaaaaggcatcaatgttgctgagaacgagcagttcaaggcagcatttgagcacctgcttaatgagttcaaaaagcgggaagagcgttactctcccaacacgctgcgccggctggaaagcgcgtgaacctgttttgttgactgatgtttggcgaatcatcgccattctttgccggcgacaccggataccgtagaagccttctttattgagcgagcggaagagttacaccgaaacacgttatcggtgtatcgctgggcgatctcccgtgttcaccgtgtggctggctgtccggatccctgcctcgatatctacgtggaagaccggctgaaagccattgctcgcaagaaagtgagagagggcgaggccgtgaaacaggcgtccccgttcaatgaacaacacctgcttaaactgacctcgctttggtaccgcagtgacaagttactgcttcgccgcaatctggcattgctggccgtggcttatgagtcaatgctgcgagcttcagagctggcgaatattcgcgtgagtgatatggagctggccggcgacggtaccgcgatactgactatccccatcaccaaaaccaatcacagcggggagccggatacctgcatactcagtcaggatgtggtcagcttgctgatggattacaccgaagccggcaagctggatatgagcagtgacggctttttgtttgtgggcgtgtccaagcacaacacctgcatcaaacccaagaaggacaagcagaccggcgaggtcctgcacaagccaatcaccaccaaaacggttgagggcgtgttctattctgcatgggaaactttggacttaggacgtcagggggtgaagcctttcacggcacacagtgcgcgtgttggcgctgctcaagatctgcttaaaaaaggctataacaccctgcagatccaacagtctggccgatggtctagcggtgccatggtggcgagatatggcagggcgatactggcgcgtgatggtgcaatggctcatagccgtgtcaaaacacgctcagcccctatgcagtggggaaaagatgaaaaggattaa

SEQ ID NO: 8 - protein, 380 aa, Vibrio parahaemolyticus
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 9 - DNA, 600 bp, Artificial Sequence
  Puromycin resistance
atgactgaatataaacctactgttagattagctactagagatgatgttcctagagctgttagaactttagctgctgcttttgctgattatcctgctactagacatactgttgatcctgatagacatattgaaagagttactgaattacaagaattatttttaactagagttggtttagatattggtaaagtttgggttgctgatgatggtgctgctgttgctgtttggactactcctgaaagtgttgaagctggtgctgtttttgctgaaattggtcctagaatggctgaattaagtggtagtagattagctgctcaacaacaaatggaaggtttattagctcctcatagacctaaagaacctgcttggtttttagctactgttggtgttagtcctgatcatcaaggtaaaggtttaggtagtgctgttgttttacctggtgttgaagctgctgaaagagctggtgttcctgcttttttagaaactagtgctcctagaaatttacctttttatgaaagattaggttttactgttactgctgatgttgaagttcctgaaggtcctagaacttggtgtatgactagaaaacctggtgcttaa

SEQ ID NO: 10 - protein, 199 aa, Artificial Sequence
  Puromycin resistance
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 11 - DNA, 315 bp, Mycoplasma pneumoniae
atgcttaaccgcgtttttttggagggtgagatagaaagttcgtgctgaagcgttaaaaaaactggatttttagtaaccattaagcaaatgcgcttttttggcgagcgtttatttactgactattacgtcatttatgccaatggtcaactggcttacgaattggagaaacacaccaagaagtacaagaccattagtattgaaggcattttgcgcacttatttagagcgtaagagtgaaatttgaaagaccacaattgaaattgtcaagattttcaatccgaaaaacgaaatagttatcgattacaaagagatctaa

SEQ ID NO: 12 - protein, 104 aa, Mycoplasma pneumoniae
MLNRVFLEGEIESSCWSVKKTGFLVTIKQMRFFGERLFTDYYVIYANGQLAYELEKHTKKYKTISIEGILRTYLERKSEIWKTTIEIVKIFNPKNEIVIDYKEI

SEQ ID NO: 13 - DNA, 114 bp, Artificial Sequence
  MOdelta50
  misc_feature (1)..(1): n is thymine wherein a phosphorothioate bond substitutes a sulfur atom for a non-bridging oxygen in the phosphate backbone
  misc_feature (2)..(2): n is cytosine wherein a phosphorothioate bond substitutes a sulfur atom for a non-bridging oxygen in the phosphate backbone
  misc_feature (3)..(4): n is thymine wherein a phosphorothioate bond substitutes a sulfur atom for a non-bridging oxygen in the phosphate backbone
nnnntgtgaataagggtggatcttactcatcaaacttccataccgttcgtataatgtatgctatacgaagttatattcgtttgccggtaaaagtgcaaagcgtttaaattttgg

SEQ ID NO: 14 - DNA, 114 bp, Artificial Sequence
  MOdelta50
  misc_feature (1)..(4): n is adenine wherein a phosphorothioate bond substitutes a sulfur atom for a non-bridging oxygen in the phosphate backbone
nnnnattgctcattttctcaaatcttttaatgaattaagctaccgttcgtataatgtatgctatacgaagttattgattatctactcaatcctaaacattcacaaggtgaagaa

SEQ ID NO: 15 - DNA, 114 bp, Artificial Sequence
  NMOdelta900
  misc_feature (1)..(4): n is adenine wherein a phosphorothioate bond substitutes a sulfur atom for a non-bridging oxygen in the phosphate backbone
nnnnattgctcattttctcaaatcttttaatgaattaagctaccgttcgtataatgtatgctatacgaagttattcatccttgtcattaaactggaagagagaaccaggatagt

SEQ ID NO: 16 - DNA, 114 bp, Artificial Sequence
  NMOdelta2kb
  misc_feature (1)..(4): n is adenine wherein a phosphorothioate bond substitutes a sulfur atom for a non-bridging oxygen in the phosphate backbone
nnnnattgctcattttctcaaatcttttaatgaattaagctaccgttcgtataatgtatgctatacgaagttattgtcagaaactgtcttgtaaatctgttagattggcaaaat

SEQ ID NO: 17 - DNA, 114 bp, Artificial Sequence
  NMOdelta4kb
  misc_feature (1)..(4): n is adenine wherein a phosphorothioate bond substitutes a sulfur atom for a non-bridging oxygen in the phosphate backbone
nnnnattgctcattttctcaaatcttttaatgaattaagctaccgttcgtataatgtatgctatacgaagttatgcaaaatggccttcttattacgtcaataataagcactgtc

SEQ ID NO: 18 - DNA, 114 bp, Artificial Sequence; identical to SEQ ID NO: 17
  NMOdelta10kb
  misc_feature (1)..(4): n is adenine wherein a phosphorothioate bond substitutes a sulfur atom for a non-bridging oxygen in the phosphate backbone

SEQ ID NO: 19 - DNA, 4107 bp, Artificial Sequence
  Modified eCas9
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttgcagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccagcacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaagcccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 20 - protein, 1368 aa, Artificial Sequence
  Modified eCas9
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - DNA, 4107 bp, Artificial Sequence
  Modified eCas9 nickase
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttgcagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccagcacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaagcccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 22 - protein, 1368 aa, Artificial Sequence
  Modified eCas9 nickase
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - DNA, 4107 bp, Artificial Sequence
  eCas9 catalytic inactivated
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgcgattgttccacaaagtttccttgcagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccagcacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaagcccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 24 - protein, 1368 aa, Artificial Sequence
  eCas9 catalytic inactivated
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 25 - DNA, 336 bp, Bacillus amyloliquefaciens
atggcacaggttatcaacacgtttgacggggttgcggattatcttcagacatatcataagctacctgataattacattacaaaatcagaagcacaagccctcggctgagtggcatcaaaagggaaccttgcagacgtcgctccggggaaaagcatcggcggagacatcttctcaaacagggaaggcaaactcccgggcaaaagcggacgaacatgacgtgaagcggatattaactatacatcaggcttcagaaattcagaccggattctttactcaagcgactgactgatttacaaaacaacggaccattatcagacctttacaaaaatcagataa

SEQ ID NO: 26 - protein, 111 aa, Bacillus amyloliquefaciens
MAQVINTFDGVADYLQTYHKLPDNYITKSEAQALGWVASKGNLADVAPGKSIGGDIFSNREGKLPGKSGRTWREADINYTSGFRNSDRILYSSDWLIYKTTDHYQTFTKIR

SEQ ID NO: 27 - DNA, 714 bp, Saccharomyces cerevisiae
atgcatatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataa

SEQ ID NO: 28 - protein, 237 aa, Saccharomyces cerevisiae
MHMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 29 - DNA, 876 bp, Homo sapiens
atgaagtccaagttgaaactcaaacgctatttactctttctccccttgttaccactcggtaccttgagtttagctaacacttacttgtgacaattaaagattgctgcttttaacattcaaacctttggtgaaaccaagatgagtaacgctaccttagtgagttacattgtgcaaattttaagtcgctacgatattgctttagtgcaagaagtgcgcgatagtcacttaaccgctgtgggtaagttattagataacttaaaccaagatgctcccgatacctaccactacgtggtgagtgaacccttaggtcgcaacagttacaaggaacgctacttatttgtgtaccgccccgatcaagtgagtgctgtggatagttactactacgatgatggttgtgaaccctgtggtaacgatacctttaaccgcgaacccgctattgtgcgcttttttagtcgctttaccgaagtgcgcgaatttgctattgtgcccttacacgctgctcccggtgatgctgtggctgaaattgatgctttatacgatgtgtacttagatgtgcaagaaaagtgaggtttagaagatgtgatgttaatgggtgattttaacgctggttgtagttacgtgcgccccagtcaatgaagtagtattcgcttatgaaccagtcccacctttcaatgattaattcccgatagtgctgataccaccgctacccccacccactgtgcttacgatcgcattgtggtggctggtatgttattacgcggtgctgtggtgcccgatagtgctttaccctttaactttcaagctgcttacggtttaagtgatcaattagctcaagctattagtgatcactaccccgtggaagtgatgttaaagtag

SEQ ID NO: 30 - protein, 291 aa, Homo sapiens
MKSKLKLKRYLLFLPLLPLGTLSLANTYLWQLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 31 - DNA, 47 bp, Artificial Sequence
  Primer
acggtatcgataagcttgataaaaaatacctgagtctttcaggtatt

SEQ ID NO: 32 - DNA, 48 bp, Artificial Sequence
  Primer
tacctgagtctttcaggtatttttttttaagacccactttcacattta

SEQ ID NO: 33 - DNA, 31 bp, Artificial Sequence
  Primer
tttttttcatatgctctatcaatgatagagg

SEQ ID NO: 34 - DNA, 35 bp, Artificial Sequence
  Primer
gatagagcatatgaaaaaaaataaaggacttaacg

SEQ ID NO: 35 - DNA, 42 bp, Artificial Sequence
  Primer
tctaaatactagaattcgatttaactgtttgtttcactgtct

SEQ ID NO: 36 - DNA, 40 bp, Artificial Sequence
  Primer
acggtatcgataagcttgatcttgtactgccttttggtgc

SEQ ID NO: 37 - DNA, 40 bp, Artificial Sequence
  Primer
gtatttagaattaataaagtgcaagaacagcaagccaaac

SEQ ID NO: 38 - DNA, 41 bp, Artificial Sequence
  Primer
actttattaattctaaatactataccgttcgtatagcatac

SEQ ID NO: 39 - DNA, 40 bp, Artificial Sequence
  Primer
actacgagcgaaaaaccgcataccgttcgtataatgtatg

SEQ ID NO: 40 - DNA, 20 bp, Artificial Sequence
  Primer
tgcggtttttcgctcgtagt

SEQ ID NO: 41 - DNA, 41 bp, Artificial Sequence
  Primer
ccgggctgcaggaattcgatcctaaaggcaaggacattctt

SEQ ID NO: 42 - DNA, 20 bp, Artificial Sequence
  Primer
  misc_feature (1)..(1): n is cytosine wherein a phosphorothioate bond substitutes a sulfur atom for a non-bridging oxygen in the phosphate backbone
  misc_feature (2)..(3): n is thymine wherein a phosphorothioate bond substitutes a sulfur atom for a non-bridging oxygen in the phosphate backbone
  misc_feature (4)..(4): n is guanine wherein a phosphorothioate bond substitutes a sulfur atom for a non-bridging oxygen in the phosphate backbone
nnnntactgccttttggtgc

SEQ ID NO: 43 - DNA, 21 bp, Artificial Sequence
  Primer
  misc_feature (1)..(1): biotinylated cytosine
nctaaaggcaaggacattctt

SEQ ID NO: 44 - protein, 4 aa, Artificial Sequence
  Linker
GGGS

SEQ ID NO: 45 - protein, 5 aa, Artificial Sequence
  Linker
GGGGS

SEQ ID NO: 46 - protein, 5 aa, Artificial Sequence
  Linker
GGGGG

SEQ ID NO: 47 - protein, 5 aa, Artificial Sequence
  Linker
EAAAK

SEQ ID NO: 48 - protein, 5 aa, Artificial Sequence
  Linker
EAAAR

SEQ ID NO: 49 - protein, 6 aa, Artificial Sequence
  Linker
AEAAAK

SEQ ID NO: 50 - protein, 5 aa, Artificial Sequence
  Linker
PAPAP

SEQ ID NO: 51 - protein, 4 aa, Artificial Sequence
  Linker
GFLG

SEQ ID NO: 52 - protein, 4 aa, Artificial Sequence
  Linker
GSAT

SEQ ID NO: 53 - DNA, 79 bp, Artificial Sequence
  CmONsense
  misc_feature (1)..(2): n is thymine wherein a phosphorothioate bond substitutes a sulfur atom for a non-bridging oxygen in the phosphate backbone
  misc_feature (3)..(3): n is guanine wherein a phosphorothioate bond substitutes a sulfur atom for a non-bridging oxygen in the phosphate backbone
  misc_feature (4)..(4): n is thymine wherein a phosphorothioate bond substitutes a sulfur atom for a non-bridging oxygen in the phosphate backbone
nnnnacaccgttttccatgagcaaactgaaacgttttcatcgctctggagtgaataccacgacgatttccggcagtttc

SEQ ID NO: 54 - DNA, 80 bp, Artificial Sequence
  CmONantisense
  misc_feature (1)..(1): n is guanine wherein a phosphorothioate bond substitutes a sulfur atom for a non-bridging oxygen in the phosphate backbone
  misc_feature (2)..(4): n is adenine wherein a phosphorothioate bond substitutes a sulfur atom for a non-bridging oxygen in the phosphate backbone
nnnnctgccggaaatcgtcgtggtattcactccagagcgatgaaaacgtttcagtttgctcatggaaaacggtgtaacaa